(19) United States
(12) Patent Application Publication
(10) Pub. No.: US 2010/0179573 A1
(43) Pub. Date: Jul. 15, 2010

(54) MEDICAL DEVICE AND PROCEDURE FOR ATTACHING TISSUE TO BONE

(75) Inventors: David Gordon Levinsohn, San Diego, CA (US); Alan B. Miller, Jamison, PA (US); Shawn T. Huxel, Lawrenceville, NJ (US); Richard Thomas Briganti, Philadelphia, PA (US)

Correspondence Address: Saul Ewing LLP (Philadelphia), Attn: Patent Docket Clerk, 2 North Second St., Harrisburg, PA, 17101, US

(73) Assignee: Core Essence Orthopaedics, LLC, Yardley, PA (US)

(21) Appl. No.: 12/729,769

(22) Filed: Mar. 23, 2010

Related U.S. Application Data
(63) Continuation-in-part of application No. 12/297,530, filed on Jul. 23, 2009.
(60) Provisional application No. 60/855,828, filed on Oct. 31, 2006.
(60) Provisional application No. 60/855,831, filed on Oct. 31, 2006.
(60) Provisional application No. 60/922,558, filed on Apr. 9, 2007.

(51) Int. Cl. A61B 17/04

(52) U.S. Cl. 606/145; 606/151; 606/232

(57) ABSTRACT

The invention pertains to medical devices for anchoring a suture engaged with soft tissue to a bone, the devices including one or more of tissue fastening medical devices, bone anchor medical devices, bone anchor driving tools, implantation tools, and impactor tools, and procedures for using the same.

### RELATED APPLICATIONS

This application claims priority as a continuation in part of U.S. patent application Ser. No. 12/297,530, which is a national stage filing of PCT Application No. PCT/US07/23108, which further claims priority to U.S. Provisional Patent Application Nos. 60/855,831 filed Oct. 31, 2006, 60/855,828 filed Oct. 31, 2006, and 60/922,558 filed Apr. 9, 2007, all of which are incorporated herein by reference in their entireties.

### FIELD OF THE INVENTION

The invention relates to medical devices and procedures for attaching tissue to bone.

The invention relates particularly to medical devices and to medical procedures incorporating the use of the medical devices, that can be used in the repair of tendon tears and the like, where repair requires the reattachment of soft tissue to skeletal structures, i.e. bones.

### BACKGROUND OF THE INVENTION

Rotator cuff tears often require reattachment of soft tissue to skeletal structures and the explanation of the invention as hereinafter set out refers particularly to the repair of rotator cuff injuries, although it must be understood that the invention can be employed also in association with other like injuries where similar repair techniques are ordinarily employed or considered. The rotator cuff is the anatomical term given to a group of muscles and their tendons that act to move and stabilize the shoulder. These muscles extend from the scapula, i.e. the shoulder blade bone, and connect to the humerus, i.e. the upper arm, via their tendons, forming a cuff at the shoulder joint, thus serving to control different arm movements. A rotator cuff tear can result from a trauma to a shoulder or through wear and tear and be associated with one or more tendons becoming torn, leading to pain, shoulder instability and/or restricted arm movement.

Rotator cuff repair involves a surgeon reattaching each damaged tendon to the humerus. The conventional surgical process typically includes the steps of gaining access to the injured rotator cuff by making an incision in the shoulder and splitting the deltoid muscle and then removing scar tissue that has built up on each torn tendon. The surgeon then creates a trough at the top of the humerus and drills small holes through the bone, whereafter he sews the tendon to the bone with sutures passing through the holes. Other steps also may be associated with the process in order to deal with specific repair requirements. Following the process, the arm is incapacitated and healing is allowed to occur, which involves the reattachment of the tendons to the bone and which is generally a slow process.

Instead of passing sutures through holes drilled in the humerus for securing the tendon to the humerus, it is also known to use permanent anchors with sutures attached, inserted in the humerus, for this purpose.

More recently, arthroscopic surgery is being employed for rotator cuff repair. The surgery is performed through one or more small incisions. The surgeon observes the area of interest via a display screen which displays live images from a camera that is placed in a tube (cannula) passing through a small incision into the joint space. The instruments used are thin and are contained in separate cannulas that are inserted into the shoulder via separate small incisions. This arthroscopic surgery process includes placing anchor devices to which sutures are engaged for securing tendons to the humerus. In some techniques a pilot hole is required prior to placement of an anchor device. Each suture is passed through the tendon with a suture passing instrument. In most cases, all of the sutures are passed before tying. The sutures are then tied to anchor devices by the technique of arthroscopic knot tying. Various difficulties are associated with arthroscopic surgery as above envisaged.

The location of and the angle of a pilot hole for an anchor device is difficult to appreciate arthroscopically, rendering the location of anchor devices in their holes difficult.

The tying of sutures arthroscopically is very challenging.

Insofar as suture management is concerned, present techniques often require multiple sutures to be placed in position first and then to be tied to their anchor devices, often creating a “spider web” with entanglement of sutures and resulting in accidental pull-out of sutures and failure to recognize appropriate suture strands to be tied. Placing of sutures also presents difficulties insofar as multiple passes through the tendon are often required and snaring of suture portions by the soft tissue forming a tendon also can occur, resulting in difficulty in retrieving sutures into the portal of the equipment used.

### SUMMARY OF THE INVENTION

The invention pertains to medical devices for anchoring a suture engaged with soft tissue to a bone, the devices including tissue fastening medical devices, bone anchor medical devices, bone anchor driving tools, implantation tools, and impactor tools, and procedures for using the same. The invention may also be used to anchor tissue directly to a bone without the use of intervening sutures. For instance, the invention may be used to directly anchor ligaments and tendons to bones.

### BRIEF DESCRIPTION OF THE DRAWINGS

Further features of the various aspects of the invention are described hereinafter with reference to the accompanying diagrammatic drawings. In the drawings:

FIG. 1 shows a side view of an anchor main body forming a part of a first embodiment of a bone anchor device for anchoring a suture engaged with soft tissue to a bone in accordance with the invention;

FIG. 2 shows a top view of the anchor main body of FIG. 1;

FIG. 3 shows a cross-sectional side view of the anchor main body of FIG. 1, along line III-III of FIG. 2;

FIG. 4 shows a side view of an eyelet pin forming a further part of the first embodiment of the bone anchor device of which the anchor main body of FIG. 1 forms a part, the eyelet pin being configured to cooperate with the anchor main body of FIG. 1;

FIG. 5 shows a perspective view of a tissue fastener device for use in a medical procedure associated with the use of a medical device including the anchor main body of FIG. 1 and the eyelet pin of FIG. 4;

FIG. 6 shows a top view of the tissue fastener medical device of FIG. 5;

FIG. 7 shows a side view of the tissue fastener medical device of FIG. 5;

FIGS. 8 to 12 schematically illustrate steps associated with a first medical procedure for attaching soft tissue to bone and which includes the use of the bone anchor device FIGS. 1-4 and the tissue fastener device of FIGS. 5-7;

FIGS. 13 to 15 schematically illustrate steps associated with a second medical procedure for attaching soft tissue to bone and which includes the use of the bone anchor device of FIGS. 1-4 and the tissue fastener device of FIGS. 5-7;

FIG. 16 illustrates schematically a third procedure for attaching soft tissue to bone and which includes the use of the tissue fastener device of FIG. 5;

FIGS. 17 and 18 illustrate schematically the steps associated with a fourth procedure for attaching soft tissue to bone and which includes the use of the tissue fastener device of FIG. 5, a suture, and a conventional bone anchor;

FIG. 19 shows a cross-sectional side view of an anchor main body forming a part of a second embodiment of a bone anchor device for anchoring a suture engaged with soft tissue to a bone in accordance with the invention;

FIG. 20 shows a side view of an eyelet pin forming a part of the second embodiment of the bone anchor device for anchoring a suture engaged with soft tissue to a bone of which the anchor main body of FIG. 19 forms a part, the eyelet pin being configured to cooperate with the anchor main body of FIG. 19;

FIG. 21 shows a side view of an anchor main body forming a part of a third embodiment of a bone anchor device for anchoring a suture engaged with soft tissue to a bone in accordance with the invention;

FIG. 22 shows a top view of the anchor main body of FIG. 21;

FIG. 23 shows a cross-sectional side view of the anchor main body of FIG. 21, along VII-VII of FIG. 22;

FIG. 24 shows a side view of an eyelet pin forming a part of the third embodiment of the bone anchor device for anchoring a suture engaged with soft tissue to a bone of which the anchor main body of FIG. 21 forms a part, the eyelet pin being configured to cooperate with the anchor main body of FIG. 21;

FIG. 25 shows the bone anchor device of FIGS. 19-24 in the closed state.

FIG. 26 shows a cross-sectional side view of an anchor main body and eyelet pin of the third set of embodiments of a medical device for anchoring a suture engaged with soft tissue to a bone in accordance with the invention, the pin being located in its closed configuration within a receiving formation defined by the anchor main body;

FIGS. 27 and 28 illustrate schematically a fifth procedure for attaching soft tissue to a bone and which includes the use of the second embodiment of the bone anchor device as illustrated in FIGS. 19 and 20;

FIGS. 29 and 30 illustrate schematically a sixth procedure for attaching soft tissue to a bone and which includes the use of both the second embodiment of the medical device as illustrated in FIGS. 19 and 20 and the third embodiment of the medical device as illustrated in FIGS. 21 to 24;

FIGS. 31 and 32 illustrate a variation of the procedure illustrated in FIGS. 29 and 30 in accordance with the invention;

FIGS. 32 to 35 illustrate three further procedures for attaching soft tissue to a bone and which include the use of bone anchor device in accordance with the invention;

FIG. 36 shows a cross-sectional side view of a bone anchor device in the open state in accordance with a fourth embodiment of the invention for anchoring a suture engaged with the soft tissue to a bone;

FIG. 37 shows a cross-sectional side view of the bone anchor device of FIG. 36 in the closed state;

FIG. 38 shows a perspective view of the anchor main body portion of the bone anchor device of FIG. 36;

FIG. 39 shows a side view of the eyelet pin of the bone anchor device of FIG. 36;

FIG. 40 shows a perspective view of a C-ring that can be employed as the locking ring of the bone anchor device of FIG. 36;

FIG. 41 shows a perspective view of the retainer of the bone anchor device of FIG. 36;

FIG. 42 shows a cross-sectional side view of a bone anchor device in the open state in accordance with a fifth embodiment of the invention;

FIG. 43 is a cross-sectional side view of the bone anchor device of FIG. 42 in the closed state;

FIG. 44A shows a close-up view of the eyelet of the eyelet pin in accordance with a first alternate embodiment of the bone anchor device of FIG. 36 in the open state (sixth set of embodiments);

FIG. 44B shows a close-up view of the eyelet of the eyelet pin in accordance with the first alternate embodiment of the bone anchor device of FIG. 36 in the closed state;

FIG. 44C shows a close-up view of the eyelet of the eyelet pin in accordance with a second alternate embodiment of the bone anchored device of FIG. 36 in the open state;

FIG. 44D shows a close-up view of the eyelet of the eyelet pin of the bone anchor device in accordance with the second alternate embodiment of FIG. 36 in the closed state;

FIG. 44E shows a perspective view of the cylinder of the second alternative embodiment of FIGS. 44C and 44D separate from the overall device.

FIG. 44F shows a further embodiment of the eyelet pin of the bone anchor device in cross-section in the closed state;

FIG. 45 is a semi-transparent perspective view of a driver for driving a bone anchor device into bone in accordance with an embodiment of the present invention;

FIG. 46 is a semi-transparent side view of an impactor tool for driving the center pin of a bone anchor device of the present invention from the open position to the closed condition in the anchor main body of the bone anchor device;

FIG. 47 is a close-up, semi-transparent view of the proximal end of the impactor tool of FIG. 46;

FIG. 48 is a close-up, semi-transparent view of the distal end of the impactor tool of FIG. 46;

FIG. 49 shows a perspective view of an alternate locking ring to that illustrated in FIG. 40;

FIG. 50 shows a cross-sectional side view of a bone anchor device in the open state in accordance with a seventh embodiment of the invention;

FIG. 51 shows a cross-sectional side view of a bone anchor device in the closed state in accordance with the seventh embodiment of the invention shown.

FIG. 52A shows a perspective view of an eyelet pin in accordance with an eighth set of embodiments of the invention;

FIG. 52B shows a top plan view of the eyelet pin in accordance with the eight set of embodiments of the invention;

FIG. 53 shows a cross-sectional side view of the anchor main body in accordance with the eighth set of embodiments of the invention;

FIG. 54A shows a perspective view of a bone anchor device and corresponding implantation device in accordance with the eighth set of embodiments of the invention;

FIG. 54B shows a cross-sectional side view of the bone anchor device and corresponding implantation device in accordance with the eighth set of embodiments of the invention;

FIG. 55A shows a cross-sectional side view of the proximal end of the implantation device in accordance with the eighth set of embodiments of the invention;

FIG. 55B is an exploded view of the proximal end of the implantation device in accordance with the eighth set of embodiments of the invention;

FIG. 56A is an exploded view of the distal end of the implantation device with the implantable bone anchor in accordance with the eighth set of embodiments of the invention;

FIG. 56B shows a cross-sectional side view of the distal end of the implantation device and the implantable bone anchor taken through section B-B in FIG. 55A;

FIG. 56C shows a cross-sectional side view of the distal end of the implantation device and the proximal portion of the implantable bone anchor taken through section C-C in FIG. 55A; FIG. 57A is a perspective view of an implantation device including the implantable device and a suture shuttle in accordance with a ninth set of embodiments;

FIG. 57B is a perspective view of a suture shuttle in accordance with the ninth set of embodiments;

FIG. 57C is a close up view showing the ends of alternative suture shuttles in accordance two alternate embodiments of a suture shuttle;

FIG. 57D is a close up perspective view of the proximal end of the implantation tool handle in accordance with the ninth set of embodiments showing an alternative aperture arrangement;

FIG. 57E shows the tool of FIG. 57A during a first stage in which sutures are being loaded into the suture shuttle;

FIG. 57F shows the tool of FIG. 57A during a second stage in which sutures are being loaded into the suture shuttle;

FIG. 57G shows the tool of FIG. 57A during a third stage in which sutures are being loaded into the suture shuttle;

FIG. 57H shows the tool of FIG. 57A during a fourth stage in which sutures are being loaded into the suture shuttle;

FIG. 57I shows the tool of FIG. 57A during a fifth stage in which sutures are being loaded into the suture shuttle;

FIG. 57J shows the tool of FIG. 57A during a sixth stage in which sutures are being loaded into the suture shuttle;

FIG. 57K is a cross-sectional side view of the handle of the implantation tool in accordance with the ninth set of embodiments illustrating an alternate embodiment including a cap;

FIG. 58 is a close up view of the suture shuttle in accordance with the ninth set of embodiments passing through the eyelet of the eyelet pin;

FIG. 59 is a perspective view of the implantation tool in accordance with the ninth set of embodiments bearing a protective sheath;

FIGS. 60A-60C illustrate an alternate embodiment of the proximal portion of an implantation tool at various stages of use in accordance with the ninth set of embodiments;

FIGS. 61A and 61B illustrate yet another alternate embodiment of the proximal portion of an implantation tool in accordance with the principles of the ninth set of embodiments;

FIG. 61C illustrates one more alternate embodiment of the proximal portion of an implantation tool in accordance with the ninth set of embodiments;

FIG. 62 is a top plan view of an exemplary bone anchor in accordance with a tenth set of embodiments;

FIG. 63 is a side cross-sectional view of another exemplary bone anchor in accordance with the tenth set of embodiments;

FIG. 64A is a perspective view of an exemplary adjustment/redeployment tool that may be used to adjust or redeploy a bone anchor in accordance with the principles of the present invention;

FIG. 64B is another perspective view of the tool of FIG. 64A with the handles removed; and

FIG. 65 is a cross-sectional side view of a bone anchor device in accordance with an eleventh set of embodiments of the invention.

### DETAILED DESCRIPTION

First Set of Exemplary Embodiments

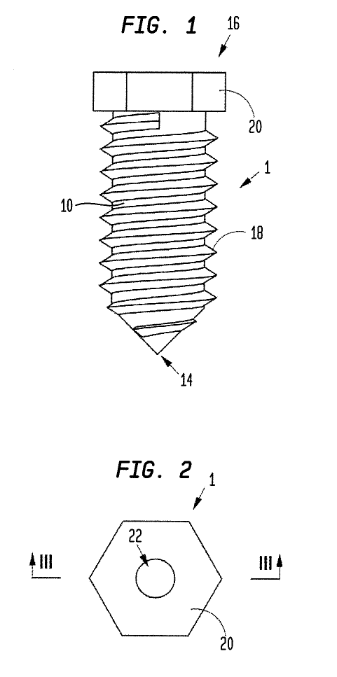

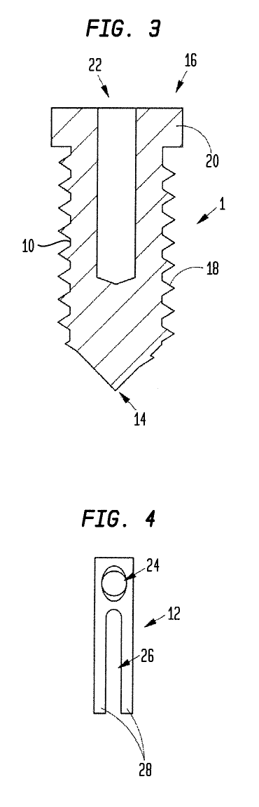

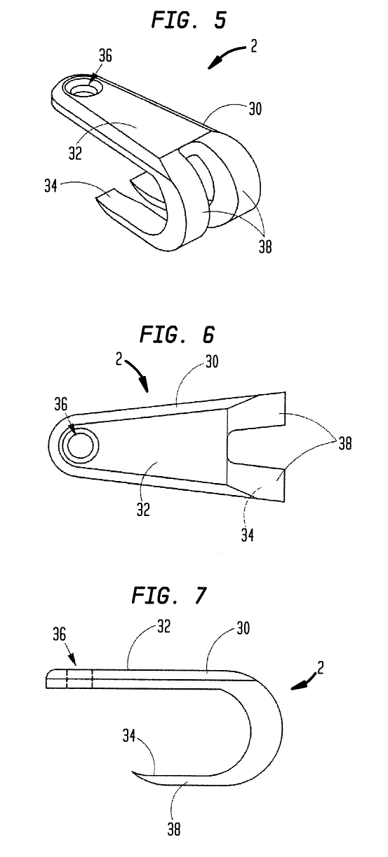

A medical system in accordance with a first embodiment of the present invention comprises two primary components, namely, a bone anchor device 1 as shown in FIGS. 1-4 and a tissue fastener device 2 as shown in FIGS. 5-7.

Referring initially to FIGS. 1-4, a bone anchor device 1 in accordance with the invention is shown for anchoring a suture that is engaged with soft tissue to a bone. It includes a substantially cylindrical body 10 and an eyelet pin 12. Both the anchor main body 10 and the eyelet pin 12 may be formed of a biocompatible material, such as of a type already commonly used within the body of a person, e.g., a metal or metal alloy such as titanium, stainless steel or cobalt-chrome alloys; a suitable polymeric material that is nonabsorbable, such as polyethylene, poly-ether-ether-ketone (PEEK), poly-ether-aryl-ketone (PEAK); a resorbable polymer selected from homopolymers, copolymers and blends of polylactide, polyglycolide, polyparadioxanone, polytrimethylene carbonate or polycaprolactone; or composites of the aforementioned with biocompatible inorganic substances such as carbon, hydroxyapatite, beta tricalcium phosphate, other calcium phosphate ceramics or calcium sulfate.

The anchor main body 10 defines a leading end 14 and a trailing end 16 and an external formation such as a thread 18 extending externally along the length thereof from its leading end towards its trailing end to help secure the body 10 to bone. At its trailing end 16, the body 10 defines a head formation 20, the head formation 20 being geometrically profiled to permit engagement with a screw driver-type tool, for screwing the body into a bone. The body 10 also defines a receiving formation therein that is in the form of a cylindrical blind bore 22, the receiving formation 22 being particularly configured to frictionally receive the eyelet pin 12 therein.

The eyelet pin 12 could be formed of the same material as the anchor main body 10, the pin comprising a substantially cylindrical pin that defines a passage 24 therethrough near a proximal end thereof and a longitudinal slot 26 that extends therein from the distal end toward the proximal end near which the passage 24 is defined. The pin thus defines two legs 28 on opposite sides of the slot 26. The pin 12 is particularly configured to be securely locatable within the receiving formation 22 defined by the anchor main body 10 by a friction fit, inherent resilient deformability of the material forming the pin and the configuration of the slot serving to enhance required location of the pin within the receiving formation 22 defined by the body 10. The exact configurations of the anchor main body and of the pin are greatly variable.

FIGS. 5-7 illustrate a tissue fastener device 2 for use in conjunction with the bone anchor device 1 in a medical process. The tissue fastener device 2 comprises a body 30 that defines a shank portion 32 and a hook formation 34, the shank portion 32 having a hole 36 defined therein near the free end thereof. Generally, the configuration of the hook formation is greatly variable, the hook formation 34 in this case being defined by two spaced apart prongs 38, the free ends of the prongs extending substantially parallel to the shank portion 32. The hole 36 permits a fastener such as a length of suture or a screw to be attached to the body 30, whereas the free end of the shank portion 32, possibly in conjunction with the location of the hole 36, is configured to be engageable with an applicator tool whereby the body can be manipulated for engaging soft tissue via the hook formation 34, within a medical procedure, as is explained in more detail hereafter.

Insofar as the tissue fastener 2 is configured for use in an arthroscopic procedure, the end region of the shank portion 32 of the body 30 where the hole 36 is defined is configured to engage an engagement formation of an applicator tool, the applicator tool providing for manipulation of the tissue fastener device 2 for engaging soft tissue, particularly via a cannula located in an incision in a body of a person in a location where it provides access to the location where the tissue fastener device 2 must be engaged with soft tissue. Although not essential, it is envisaged that such an applicator tool can be cannulated to provide for a suture to pass through the cannula, thus to provide for the free end of a suture tied to the tissue fastener device 2 to remain conveniently accessible externally of the body of a person following engagement of the device with soft tissue, as is described in more detail hereafter.

It must be understood that a specific arthroscopic applicator tool will be provided for use with the tissue fastener device 2 and/or that the tissue fastener device 2, as described, may require modification for cooperating with a particular tool, in order to facilitate its use as hereafter described.

The tissue fastener device 2 may be formed of a metal material of a type already used for medical devices used within the body of a person, e.g., a metal or metal alloy such as titanium, stainless steel and cobalt-chrome alloys; a suitable polymeric material that is nonabsorbable, such as polyethylene, poly-ether-ether-ketone (PEEK), poly-ether-aryl-ketone (PEAK); a resorbable polymer selected from homopolymers, copolymers and blends of polylactide, polyglycolide, polyparadioxanone, polytrimethylene carbonate or polycaprolactone; or composites of the aforementioned with biocompatible inorganic substances such as carbon, hydroxyapatite, beta tricalcium phosphate, other calcium phosphate ceramics or calcium sulfate.

First Set of Exemplary Surgical Procedures

The bone anchor device 1 and the tissue fastener device 2 are configured particularly for use in a medical procedure for anchoring sutures engaged with soft tissue to a bone, thereby attaching the soft tissue to the bone. Sutures engaged with soft tissue to be anchored to a bone within the procedure may be engaged with the soft tissue by any known method, although for the first procedure described hereafter with reference to FIGS. 8 to 12, the sutures are separately tied to the tissue fastener devices 2 of FIGS. 5 to 7 that are engaged with soft tissue through the engagement of the hook formations 34 of the devices 2 with the soft tissue.

The procedure as above envisaged is typically applied in association with rotator cuff repair and is hereinafter described in association with such a repair procedure, although it must be appreciated that the medical devices 1 and 2 as above described also can be used in association with other procedures that require soft tissue to be attached to or re-attached to skeletal structures, i.e., to bone.

Rotator cuff repair is required where a tendon that acts to stabilize the shoulder has torn and thus is to be reattached to the humerus, i.e. the upper arm bone, thereby to re-establish normal arm movement. As envisaged above, such repair ordinarily involves a surgeon gaining access to the tendon and the humerus through incision, engaging sutures to the tendon in a conventional manner, and then sewing the sutures to the humerus via holes formed therein for anchoring to the humerus. Anchoring to the humerus by tying the sutures to anchor devices located in the humerus also is known. The same principles apply also to the procedure that is explained hereafter with particular reference to FIGS. 8 to 12 and that is associated with the use of the medical devices 1 and 2 described above.

Referring now to FIGS. 8 to 12 of the drawings, the rotator cuff repair procedure illustrated particularly is an arthroscopic procedure which includes, as a first step, providing access to the damaged tendon 40 and the humerus 42 by forming one or more incisions in the shoulder region and inserting a cannula 44 in each incision.

The general procedure in association with the location of cannulas 44, which can provide access to required locations to permit the repair procedure to be carried out, is already well known and is thus not described further herein. Each cannula located in an incision provides access to locations where the procedure must be performed, particularly also for arthroscopic tools or instruments that can serve to suitably manipulate the medical devices above described, within the procedure. The configuration of such arthroscopic tools or instruments are generally well known, but insofar as existing tools or instruments may not be specifically configured to accommodate manipulation of the medical devices described, existing tools or implements may be suitably adapted or new tools or instruments may be designed, using known principles, in order to facilitate the procedure.

With reference to FIG. 8, the first step in the arthroscopic procedure for performing a rotator cuff repair following the location of a cannula that provides access to the humerus 42 provides for the anchor main body 10 of the bone anchor device 1 to be screwed into the humerus 42 in a desired anchoring location. An arthroscopic screw driver engaging the head formation 16 of the anchor main body 10 is used for this purpose, the typical location of the anchor main body being shown in FIG. 8 of the drawings, which also illustrates the head formation 16 of the body that remains exposed externally of the humerus 42. For a medical device having an anchor main body without a head formation, this exposure may not occur. It must be understood in relation to this procedure that a further cannula accommodates an instrument carrying a camera, enabling a surgeon to observe the area of interest, particularly via live images displayed on a display screen. Additional anchor main bodies 10 that can form anchors for sutures will be similarly screwed into the humerus 42 before proceeding with the next step in the procedure.

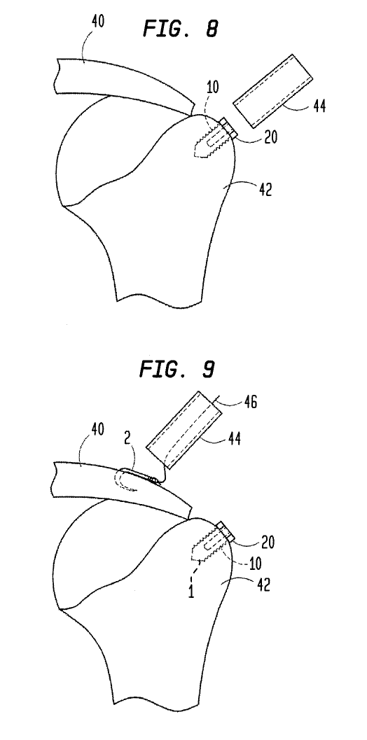

With reference to FIG. 9, the next step in the procedure provides for tying of individual sutures 46 to the respective bodies 30 of the tissue fastener devices 2. Alternately, the sutures can be pre-tied to the fastener, or simply looped through hole 36. Next, the hook formation 34 defined by each body is fastened to, under arthroscopic visualization, the tendon 40 being repaired, particularly again via a suitably located cannula 44 and with the aid of a suitable instrument that permits manipulation of the body 30 to provide for engagement of the hook formation 34 with the tendon. The suture 46 tied to each body 30 optionally may extend centrally through the applicator tool utilized, the free end of the suture thus remaining accessible externally of the person's body. A suture 46 extending from a body 30 and via a cannula to a location externally of the body is illustrated. The number of sutures engaged with the tendon 40 for its repair clearly is determined by the extent of damage to the tendon.

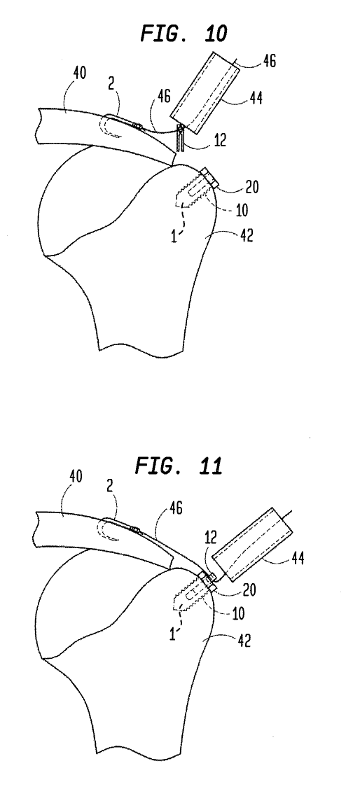

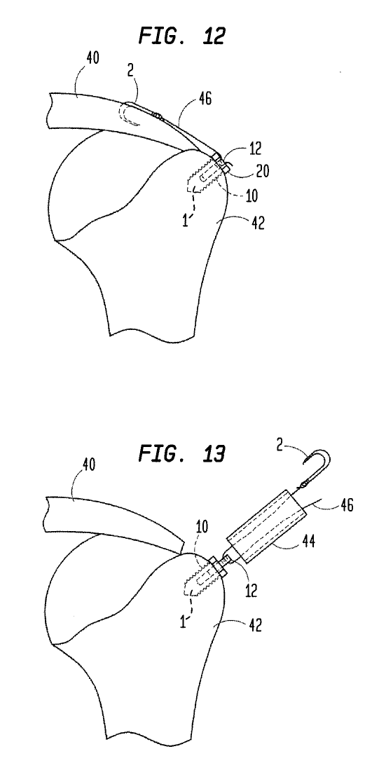

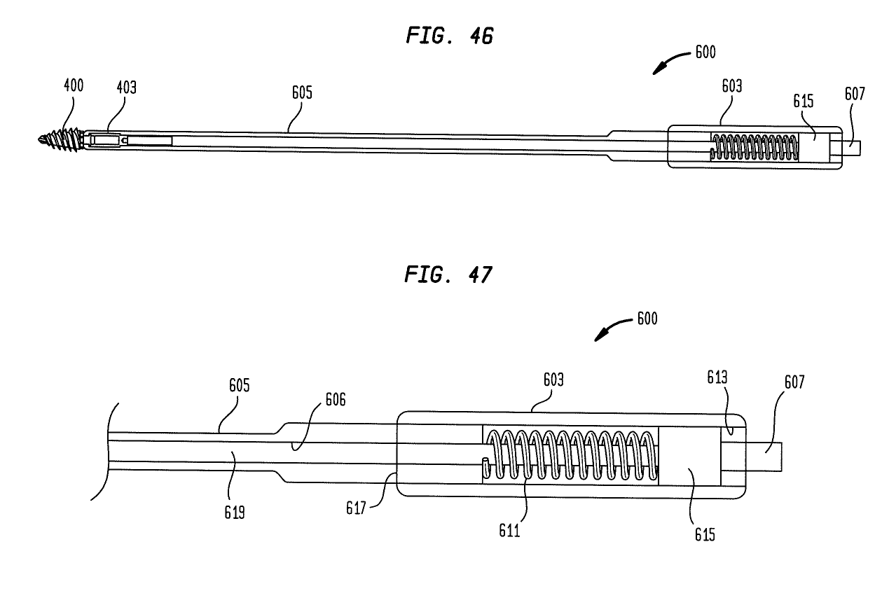

The procedure thus requires anchoring of the sutures 46 to anchor main bodies 10 via eyelet pins 12, and in this regard it must be understood that each anchor main body and its associated eyelet pin may serve to anchor either a single suture or two or more sutures with respect thereto. With reference to FIG. 10, this anchoring procedure includes, for each suture 46, threading the suture through the passage 24 defined in an eyelet pin 12, which can be done externally of the body, following which through manipulation of the eyelet pin by means of a suitable arthroscopic applicator tool such as the impactor tool shown in FIGS. 46-48 and described later, the eyelet pin is inserted through an appropriate cannula and partially inserted into the receiving formation 22 of an anchor main body 10 and the free end of the suture is pulled up through the cannula adjacent the bone anchor device 1, thus providing the configuration shown in FIG. 11. Thereafter, with reference to FIG. 12, by applying tension to the suture 46, the tendon 40 is pulled toward and against the humerus 42 from which it has been torn, thus to effectively place the tendon in abutment with the humerus in a configuration in which re-attachment with the humerus is permitted. While retaining the tension in the suture 46, the eyelet pin is further displaced into the receiving formation 22 of the anchor main body 10, particularly to the extent that the entire eyelet pin is located in the receiving formation 22. This can be achieved by impacting under arthroscopic visualization of the eyelet pin with a suitable impactor tool, such as that shown in FIGS. 46-48 and described later herein, extending through the cannula 44 and a mallet, the suture 46 being effectively anchored to the anchor main body by being clamped between the anchor main body and the pin. The free end segment of each suture can then be suitably cut-off. Normal finishing procedures associated with arthroscopic surgery can then be performed in order to finally complete the procedure.

Second Set of Exemplary Surgical Procedures

Referring now to FIGS. 13 to 15 of the drawings, a variation of the rotator cuff repair procedure as described with reference to FIGS. 8 to 12 of the drawings, is illustrated. In these Figures, like parts are designated by the same reference numerals as before. The procedure is again an arthroscopic procedure which includes, as a first step, providing access to the damaged tendon 40 and the humerus 42 by forming one or more incisions in the shoulder region and, usually, inserting a cannula in each incision. The same considerations in relation to the location of cannulas apply.

In this case, a bone anchor device 1 (including an anchor main body 10 and an eyelet pin 12) is provided in combination with at least one suture 46, threaded through the passage defined at one end of the eyelet pin 12, and a tissue fastener device 2, tied to the suture. The eyelet pin 12 is partially inserted in the receiving formation defined therefore in the anchor main body 10, free displacement of the suture 46 still being permitted.

With a cannula 44 being located that provides access to the humerus 42, the anchor main body 10 of the medical device is again screwed into the humerus in a desired anchoring location. This is achieved in the same way as before and provides the configuration shown in FIG. 13, in which the eyelet pin 12, suture 46, and body 30 are located as shown.

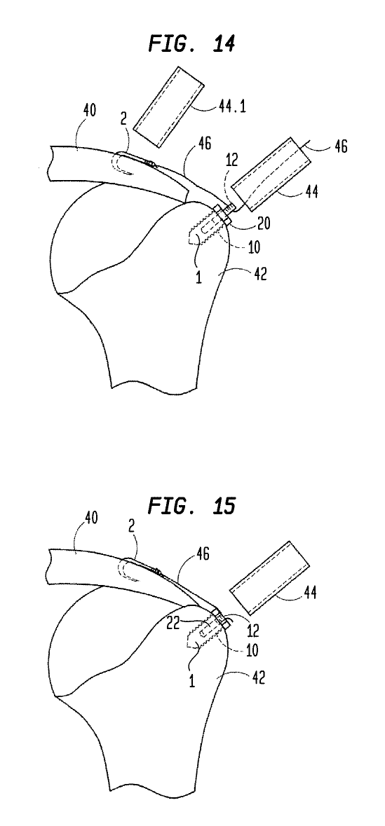

With reference to FIG. 14, the next step in the procedure provides for the suture 46 to be attached to the tendon 40 by engagement of the hook formation defined by the tissue fastener device 2 with the tendon, particularly with the aid of a suitable arthroscopic tool operated via the cannula 44.1. Instead of attachment of a suture to a tendon 40 with the aid of a tissue fastener device 2, the suture alternately can be “tied” to the tendon with the aid of a suitable suture passing instrument (not shown). Insofar as this form of attachment of a suture to a tendon is conventional and well known, it is not described or illustrated in more detail herein.

With reference to FIG. 15, with the suture 46 attached to the tendon 40, tension can be applied to the suture for displacing the tendon into its required “repair position” with respect to the humerus 42, following which the eyelet pin 12 is displaced into its fully inserted (or closed) position in its receiving formation 22 defined by the anchor main body 10, thus providing for anchoring of the suture 46 to the humerus. Following completion, the excess suture is cut-off.

It will be understood that both the above described procedures can be altered in various different respects. For example, for the procedure described with reference to FIGS. 8 to 12, it is envisaged that an eyelet pin can be partially inserted (in the open state) in an anchor main body without a suture threaded therethrough, whereafter the suture can be attached to the tendon to be repaired before being threaded through the passage 24 in the eyelet pin 12 and being anchored in position by the full insertion of the eyelet pin in its receiving formation. It must be understood in this regard that the exact procedure followed will be determined by individual procedure requirements and also the nature of the procedure which requires anchoring of sutures to bone with the aid of a medical device.

Some of the benefits associated with the use of a tissue fastener device in accordance with the invention within a medical procedure are explained hereafter particularly in relation to a rotator cuff procedure as above described, although it must be understood that some or all of these benefits may be associated also with other procedures as will be clearly apparent.

The known state-of-the-art procedures usually require placement of all sutures through the rotator cuff prior to securing of the sutures to the bone. This is necessary because the sutures are deployed into the rotator cuff tissue by a device that penetrates the full thickness of the cuff tissue; however, placement of a suture through the full thickness of the cuff tissue after a previous suture has already been secured to the bone, will potentially weaken or even disrupt the previous suture fixation. This problem cannot be resolved by moving the point of suture penetration further away from the preceding suture penetration point, as this will result in less secure fixation. One of the principle goals of rotator cuff repair is to recreate the anatomical footprint of the tendon's attachment via secure fixation and, for the reasons explained, this goal will be compromised by a “tie-as-you-go” method. It will be understood by those skilled in the art that the smaller the tear within the tendon, the less room there will be for safely placing a following suture through the torn tissue of the tendon without disrupting or weakening the prior-located suture(s).

As such, by facilitating a “tie/secure-as-you-go” procedure, the above problem of suture management is largely resolved and this is in fact achieved with the use of the tissue fastener devices of the invention, which permit “tie/secure-as-you-go” procedures. Also because the state-of-the-art procedures for the reasons explained, require multiple sutures to be engaged with rotator cuff tissue before anchoring thereof to bone, suture management of untied multiple suture strands is a major technical challenge in state-of-the-art arthroscopic rotator cuff repair. The problems intensify as the number of sutures are placed in position, a maze of sutures often leading to inadvertent tying of incorrect suture pairs, failure to find sutures in the procedure field, inadvertent release of sutures from their anchors and tangling of sutures around instruments and among other sutures and soft tissues. This suture management within the rotator cuff procedure above described and with the aid of the medical devices of the invention is greatly facilitated.

Still further, upon completion of a rotator cuff repair as envisaged, there are occasionally areas where the tendon is not adequately tensioned and not adequately laying on bone. For the reasons mentioned above, a surgeon cannot use a state-of-the-art suture passing instrument to augment the repair. However, with the use of the tissue fastener device 2 of the invention, a surgeon will have a simple option of augmenting and thereby to fine tune a repair without risking the existing repair sutures.

It is also known for suture passing devices to be used for deploying sutures into the rotator cuff. With the use of these devices there are several steps involved in the process, with each step being exposed to technical difficulties. These steps particularly involve the loading of sutures outside the portal defined by a cannula, grabbing the tendon in the jaws of the suture passing device arthroscopically, deploying the sutures arthroscopically, withdrawing the suture-passing device, and then retrieving the sutures into a portal. Alternately, cannulated suture shuttling and penetrating devices also are commonly used that involve several complex steps. Specifically, first the rotator cuff is pierced with the device. This is technically difficult, and to facilitate the procedure, devices that have various curves and or twists have been designed. Then, typically, a suture or wire (pull through stitch) is advanced through the cannulated shuttling device. This wire or suture is then retrieved into a separate cannula. Then, the suture to be used in the rotator cuff repair is placed through a loop or penetrating device in the pull-through stitch and pulled (shuttled) through the tendon. These complex processes are eliminated with the use of the tissue fastener device 2 of the invention, which affords a surgeon a simple method of attachment of suture to the tendon.

Third Set of Exemplary Surgical Procedures

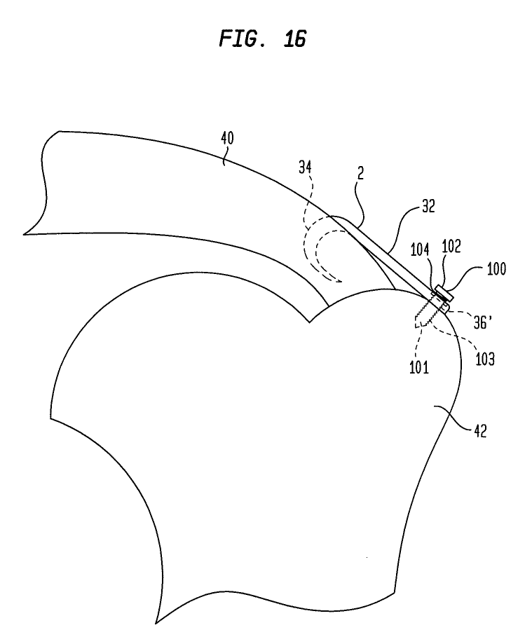

FIG. 16 illustrates an alternative surgical procedure utilizing a tissue fastener such as tissue fastener 2 in accordance with the present invention that completely eliminates the use of sutures in any form. In this embodiment, a tissue fastener device has essentially the same basic components of the tissue fastener device 2 shown in FIGS. 5-7, including a shank portion 32, a hook formation 34, and a hole 36′. Instead of threading a suture through the hole 36′, a bone anchor 100 is passed through the hole and screwed or otherwise inserted into the bone. The bone anchor 100 may be a simple bone screw with a threaded shaft 101 smaller in diameter than the diameter of the hole 36′ in the tissue fastener device 2 and a head 102 with a diameter greater than that of the hole 36′.

The hole 36′ may be counterbored (not shown) so that the head 102 of the screw 100 will be substantially flush with the surface of the shank portion 34 of the tissue fastener device 2. The screw may be polyaxial. For instance, the hole in the tissue fastener device may be spherical and the screw may have a mating spherical head so that the screw can pivot about the interface between the spherical head and the spherical seat in the hole through a defined cone of freedom. In one embodiment, the spherical head and/or the spherical seat in the hole may have ridges or other formations for interlocking with each other to generate a stronger grip between the screw head and the hole. The ridges may be plastically deformable when the screw is forced down into the seat to provide even stronger gripping there between.

In order to even further increase rigidity and help prevent backout of the bone screw 100, a mechanism to directly fixedly attach the screw 100 to the hole 36 in the tissue fastener device 2 (rather than just trapping the shank 32 of the tissue fastener device 2 between the head 102 of the screw 100 and the bone surface) may be additionally provided. For instance, hole 36′ may be internally threaded so that, when screw 100 is screwed into the bone, it also threadedly engages and becomes directly fixed to the tissue fastener device 2, not only the bone 42. In a preferred embodiment of this feature, the threads 104 on the screw 100 for engaging the hole 36′ are different than the threads 103 on the screw 100 for engaging the bone (since thread formations most suitable for threading into bone are different than thread formations most suitable for mating contact in a pre-threaded hole). In such an embodiment, the proximal portion of the shank of the screw 100 would bear threads 104 adapted for engaging the threads in the hole 36′ and the distal portion of the shank of the bone screw 100 would bear threads adapted for engaging bone.

The tissue fastener device 2 may be engaged with the soft tissue 40 in the usual fashion as discussed above in connection with FIG. 9.

Thereafter, a suitable surgical tool can be inserted through a cannula that can guide the tissue fastener to a position such that the hole 36 is positioned above the desired location on the bone for the screw 100 to be inserted. The bone screw 100 is then inserted through a cannula (not shown) into the hole 36 and screwed into the bone using a suitable driver (not shown) in order to attached the tissue fastener 2 directly to the bone without the use of sutures.

In an alternate embodiment of the tissue fastener device, the shank may include more than one hole so that the tissue fastener device can attached to the bone using multiple screws, pegs, tacks, or other bone fastening devices.

Fourth Set of Exemplary Surgical Procedures

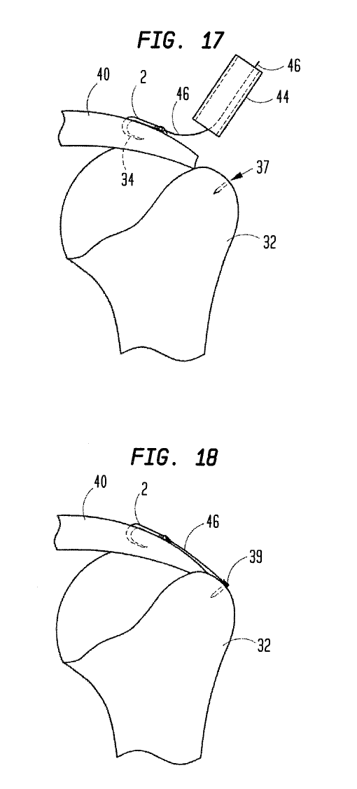

FIGS. 17 and 18 illustrate a further arthroscopic procedure for engaging a suture with soft tissue using the tissue fastener device 2 in conjunction with a conventional bone anchor 39.

Insofar as the procedure hereafter described is an arthroscopic procedure, the repair procedure is initiated by locating cannulas 44 (only one shown) in incisions that are positioned so that access is provided to the tendon 40 and the humerus 42 to which the tendon is to be attached, this access particularly accommodating the use of arthroscopic tools. The location of cannulas 44 and normal preparation in relation to a repair is conventional and, as such, is not described further herein.

Particularly, within an arthroscopic procedure as envisaged, the first step in the procedure typically involves the formation of a pilot hole 37 in the humerus 42 in a location where sutures must be anchored to the humerus. The pilot hole 37 is formed arthroscopically with the aid of a suitable tool that facilitates this. The pilot hole 37 particularly is formed to receive an anchoring device 39 therein, particularly a device to which sutures can be tied or otherwise secured for effective anchoring of the sutures to the humerus. The mode of location of an anchoring device is variable and is determined by the type of anchoring device involved, it being possible, for example, to locate an anchoring device without the requirement of first forming a drill hole.

Each suture 46 (there may be one or more) to be engaged with the tendon 40 and anchored to the anchoring device 39 to be located in the pilot hole 37 is then tied to a separate tissue fastener device 2, particularly via the hole 36 defined in the body 30 thereof. Thereafter, each body 2 is operatively engaged with an applicator tool that is configured to permit engagement of the tendon 40 by the tissue fastener device 2 via its hook formation 34, in the configuration as shown in FIG. 17. It will be understood that, when so engaged, the suture 46 tied to the device 2 will extend from the person's body via the cannula 44 through which access to the tendon is provided, the free end of the suture thus being easily “controllable”.

With each suture 46 (only one shown) engaged with the tendon 40, each suture is tied under tension to an anchoring device 39 that is then located in the pilot hole 37 provided therefore. Insofar as this anchoring procedure is already known and insofar as it does not form a part of the present invention, this is not described further herein. The above procedure is performed for each further anchoring device to be used and the sutures to be anchored thereto.

Second Set of Exemplary Embodiments

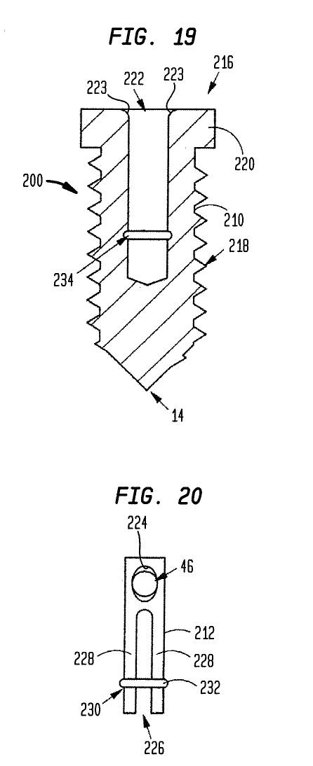

A second embodiment of the bone anchor device is shown in FIGS. 19 and 20. This bone anchor device is largely similar to the first embodiment shown in FIGS. 1-4, except for the manner and mechanism by which the eyelet pin engages the anchor body. Particularly, as in the above noted embodiments, both the anchor main body 210 and the eyelet pin 212 are formed of a metal material of a type already commonly used within the body of a person, e.g., a metal or metal alloy such as titanium, stainless steel and cobaltchrome alloys; a suitable polymeric material that is nonabsorbable such as polyethylene, poly-ether-ether-ketone (PEEK), poly-ether-aryl-ketone (PEAK); a resorbable polymer selected from homopolymers, copolymers and blends of polylactide, polyglycolide, polyparadioxanone, polytrimethylene carbonate or polycaprolactone; or composites of the aforementioned with biocompatible inorganic substances such as carbon, hydroxyapatite, beta tricalcium phosphate, other calcium phosphate ceramics or calcium sulfate.

The anchor main body 210 defines an operative leading end 214 and an operative trailing end 216 and a self-tapping thread 218 extending externally along the length thereof from its operative leading end towards its operative trailing end. At its trailing end 216 the body defines a head formation 220, the head formation being geometrically profiled to permit engagement with a screwdriver-type tool for screwing the body into a bone. The body 210 also defines a receiving formation 222 therein that is in the form of a cylindrical blind bore, the receiving formation 222 being particularly configured to securely receive an eyelet pin 212 therein.

The eyelet pin 212 defines a passage 224 therethrough near its proximal end and a longitudinal slot 226 that extends therein from the distal end. The pin thus defines two legs 228 on opposite sides of the slot 226. The pin 212 is configured to be securely locatable within the receiving formation 222 defined by the anchor main body 210, at least partially due to an effective friction fit, as in the first embodiment described above in connection with FIGS. 1-4. The inherent deformability of the material forming the pin 212 and the configuration of the slot 226 both serve to enhance the required location of the pin within the receiving formation 222 defined by the body 210. In order to further enhance the location of the pin 212 within the receiving formation 222 defined by the anchor main body 210, the pin 212 defines a peripheral groove 230 within which an elastic band, preferably an O-ring element 232, is received. The O-ring may, for instance, be made of silicone. The anchor main body 210 also defines a groove 234 within the receiving formation 222, the positioning of the grooves 230 and 234 being such that, with the pin inserted into its required operative configuration within the receiving formation 222 of the body 210, the grooves 230 and 234 will oppose one another, providing for the location of the O-ring element 232 therein, thus serving to further enhance the locking between the body and the pin when the pin is deployed downwardly into its closed position (hereinafter the “closed” position). It will be understood that the resilient elasticity of the O-ring element 232 and the slotted configuration of the pin 212 will permit the insertion of the pin 212 into the receiving formation 222 with the O-ring element effectively assembled into the groove 230, the O-ring element 232 again expanding when the grooves 230 and 234 oppose one another, as described above.

As in the first embodiment of FIGS. 1-4, with a suture 46 passing through the passage 224 and by the location of the pin 212 within the receiving formation 222 defined by the body 210, the segments of the suture extending from the section passing through the passage 224 are effectively gripped between the outer surface of the pin 212 and the inner surface of the passage 222 in the body 210, thus providing for effective anchoring of the suture, as will be explained in more detail hereafter. In order to prevent suture damage during the location of the pin 212 into the receiving formation 222 of the body 210, the end of the receiving formation 222 may be flared as shown at 223. The opposite ends of the passage 224 may be similarly flared. The formation of an effective cutting edge between the pin 212 and the body 210 is thus avoided, when the pin is inserted into the receiving formation 222 with a suture passing through the passage 224.

Third Set of Exemplary Embodiments

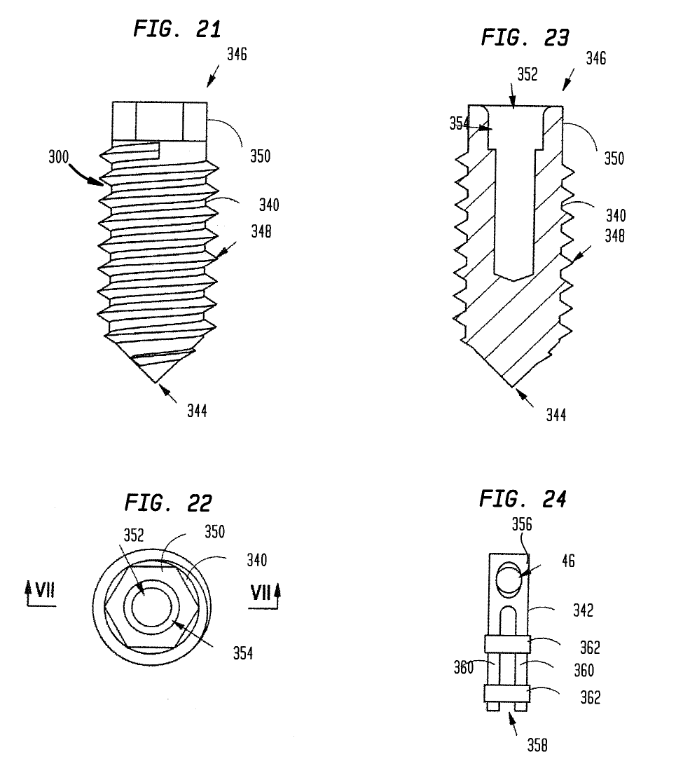

Referring now to FIGS. 21 to 25 of the drawings, a third embodiment of a bone anchor device 300 for anchoring a suture engaged with soft tissue to a bone in accordance with the invention includes a substantially cylindrical body 340 (shown in FIGS. 21-23) and an eyelet pin 342 (shown in FIG. 24). Both the anchor main body 340 and the eyelet pin 342 can be formed of materials equivalent to those referred to above. The anchor main body 340 again defines an operative leading (or distal) end 344 and an operative trailing (or proximal) end 346 and a self-tapping thread 348. At its trailing end 346, the body defines a geometrically profiled formation 50 that permits engagement with a screwdriver-type tool for screwing the body into a bone. For the purpose described hereafter, the effective diameter of the formation 350 is equal to or smaller than the diameter of the remainder of the anchor main body 340. The body 340 again defines a receiving formation 352 that is in the form of a cylindrical blind bore, the receiving formation 352, in this case, defining an enlarged trailing end segment 354, as illustrated. The receiving formation 352 provides for the secure location therein of the eyelet pin 342.

The eyelet pin 342 again defines a passage 356 therethrough near the proximal end thereof and a longitudinal slot 358 that extends therein from the distal end. The pin thus again defines two legs 360. The two legs, in this case, have bands 362 of a resiliently deformable material located thereon which, upon the location of the pin 342 in the receiving formation 352, enhance the secure location of the pin within the receiving formation.

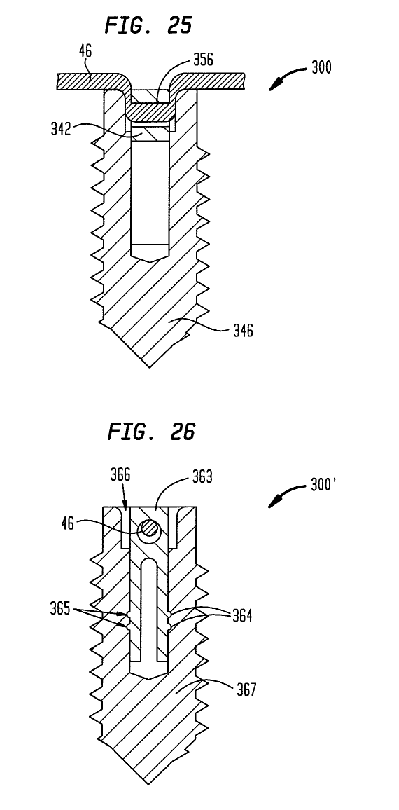

With a suture 46 passing through the passage 356 defined by the eyelet pin 342 and with the pin 342 fully inserted in the receiving formation 352 of the anchor main body 340, it will be appreciated that the suture 46 will take a tortuous path in the bone anchor device, as shown at 335 in FIG. 25, which shows the pin 342 disposed in the anchor body 342 in the closed position, particularly, insofar as the passage 356 will be located within the enlarged region 354 of the receiving formation 352.

In relation to the bone anchor devices described above, it must be appreciated that their design may vary in different respects. By way of example and with reference to FIG. 26 of the drawings, an eyelet pin 363 of a bone anchor device 300′ may define surrounding ridges 364 that are operatively located in complementary grooves 365 defined in the receiving formation 366 of the anchor main body 367 of the device, for the location of the pin in the receiving formation. This may be accommodated by the inherent resilient deformability of the material forming the pin 363. Clearly, the ridges may, alternatively, be defined within the receiving formation of the anchor main body 367 and complementary grooves may be defined within the eyelet pin 363. Any number of complementary formations may be defined for this purpose, whereas the exact configurations of these formations also are variable. Many other locating arrangements for this purpose also can be envisaged.

Fifth Set of Exemplary Surgical Procedures

The bone anchor devices 1, 200, 300, 300′, described hereinabove may be used in connection with various different procedures that involve the anchoring of soft tissue to bone, which is required in relation to the repair of various different injuries, as described hereafter. This includes any of the surgical procedures described hereinabove such as those described in connection with FIGS. 8-12 and 13-15.

It will be understood that, in relation to the anchor main body 210, the head formation 220 in the second embodiment of FIGS. 19 and 20 will protrude from the bone with the anchor main body screwed into a bone, whereas, in relation to the anchor main body 340 in the third embodiment of FIGS. 21-25, the entire anchor main body can be screwed into a bone to become effectively embedded within the bone.

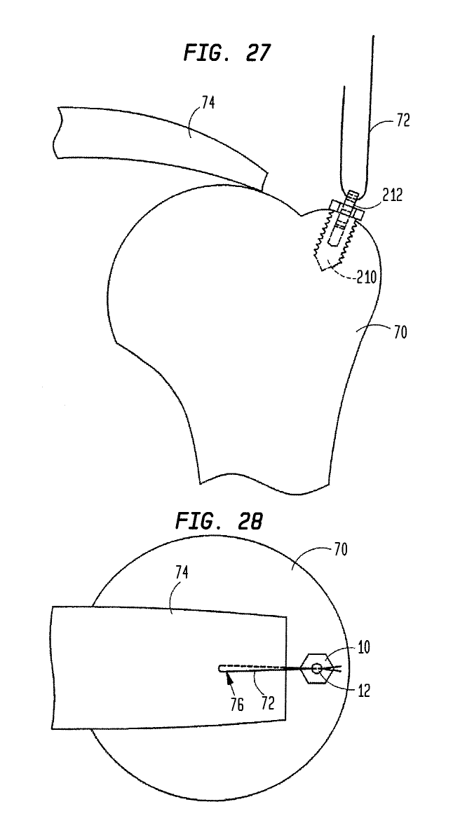

FIGS. 27 and 28 illustrate a fifth procedure or procedure step envisaged for performing a rotator cuff repair in accordance with the present invention and using the bone anchors of the present invention. FIGS. 27 and 28 illustrate this procedure utilizing the bone anchor 200 of the second embodiment illustrated in FIGS. 19 and 20 and provides for the anchor main body 210 to be screwed into the humerus 70 in a desired anchoring location. Prior to being screwed into the humerus, the anchor main body 210 has an eyelet pin 212 partially located therein, the eyelet pin 212 carrying a suture 72 as shown. It must be appreciated in this regard that the eyelet pin and suture also can be so placed immediately after the location of the anchor main body 210.

Following the location of the anchor main body 210 as shown and in order to provide for the required location of a damaged tendon 74 with respect to the humerus 70 with the aid of a suitable arthroscopic passing instrument, one end of the suture is passed through the tendon 74 and then again passed through the passage in the eyelet pin 212, thus in effect forming a closed loop 76, whereby the tendon is engaged. By thereafter applying tension to the two end segments of the suture 72, the tendon 74 is pulled towards the bone anchor device 200 into a required location with respect to the humerus 70 where re-attachment with the humerus is desired, following which the eyelet pin 212 is displaced into its closed configuration in which it is fully inserted into its receiving formation 222 defined by the anchor main body 210 to thereby effectively anchor the suture with respect to the bone anchor device 200. This position of the tendon 74 with respect to the humerus 70 is illustrated in FIG. 28, which also illustrates the loop 76 formed by the suture 72 which permits the tendon to be pulled into its required location as described. With the two ends of the suture 72 effectively gripped between the eyelet pin 212 and the anchor main body 210, required anchoring of the suture is achieved and the end segments of the suture 72 can then be cut off to provide the configuration shown in FIG. 28. It will be understood that, in relation to a particular tendon, two or more bone anchor devices 200 can be utilized, each bone anchor device being associated with the use of a suture as described. It must also be understood that, in relation to each bone anchor device used, two or more sutures may be passed through the passage of the eyelet pin thereof, wherein each suture can be passed through the associated tendon in the manner described.

It will be understood that essentially similar procedures can be performed except using the tissue fastener illustrated in FIGS. 5-7 to attach the suture to the tendon 74, rather than stitching through the tissue of the tendon.

Sixth Set of Exemplary Surgical Procedures

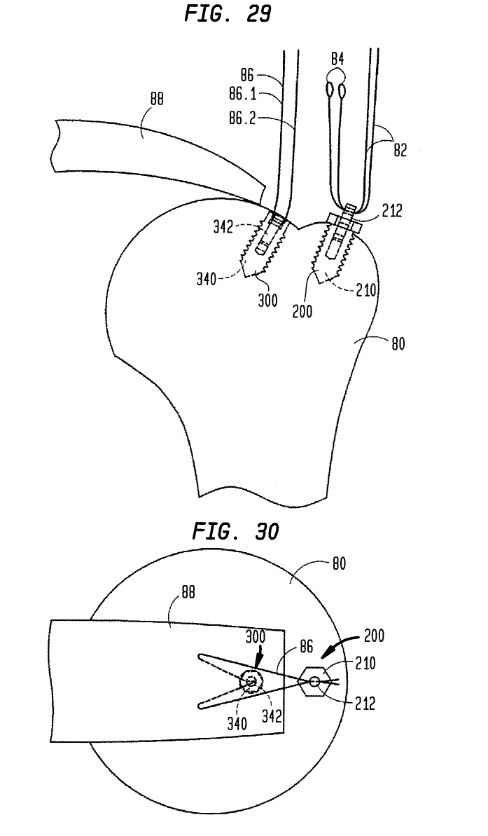

With reference to FIGS. 29 and 30 of the drawings, a sixth procedure or procedure step that is envisaged for performing a rotator cuff repair provides for an anchor main body 210 of a first bone anchor device 200 in accordance with the second embodiment as described in connection with FIGS. 19 and 20 and an anchor main body 340 of a second bone anchor device 300 in accordance with the third embodiment as described in FIGS. 21 to 24 to be screwed into the humerus 80 in locations as shown. The anchor main body 210 has an eyelet pin 212 partially located therein in the open position, the eyelet pin 212 having two separate sutures 82 passing through the passage defined by the eyelet pin 212, the sutures 82 defining loop formations 84 at one of their ends and serving as shuttling sutures as described hereafter.

The other anchor main body 340 has an eyelet pin 342 fully inserted therein, the eyelet pin 342 having a suture 86 passing through its passage. The suture 86 thus defines suture segments, 86.1 and 86.2 respectively that extend from the eyelet pin 342.

With the anchor main bodies 210 and 340 being located as shown, with the aid of a suitable passing instrument, each suture segment 86.1 and 86.2 is passed through the tendon 88 and then through a loop formation 84 in one of the shuttling sutures 82. Thereafter, by pulling on the ends of the shuttling sutures 82 remote from the loop formations 84, the shuttling sutures together with the suture segments 86.1 and 86.2, are pulled through the passage in the eyelet pin 212 of bone anchor 200, thus providing for each suture segment to form a loop that extends from the eyelet pin 342 of bone anchor 300 through the tendon 88 and back to the eyelet pin 212 of the bone anchor 200. Thereafter, by pulling on the suture segments 86.1 and 86.2, the tendon 88 is pulled towards its desired location with respect to the humerus 80 in which it should attach itself to the humerus 80, following which the eyelet pin 212 is displaced into its closed position, fully inserted in the receiving formation of the anchor main body 210. Thereby, the suture segments 86.1 and 86.2 are effectively anchored with respect to the bone anchor device 200. FIG. 30 particularly illustrates the operative configuration of the suture 86 with respect to the two bone anchor devices 200 and 300 used and a tendon 88 to be attached to the humerus 80. It must again be appreciated that further pairs of bone anchor devices 200, 300 can be utilized in a similar manner for the attachment of a tendon to a humerus, thus providing a more effective attachment footprint that will ensure the effective attachment of a tendon to a humerus.

In alternate embodiments, this surgical technique can be practiced with medial bone anchors of other designs, including conventional designs, than the bone anchor 300 of the present invention.

Seventh Set of Exemplary Surgical Procedures

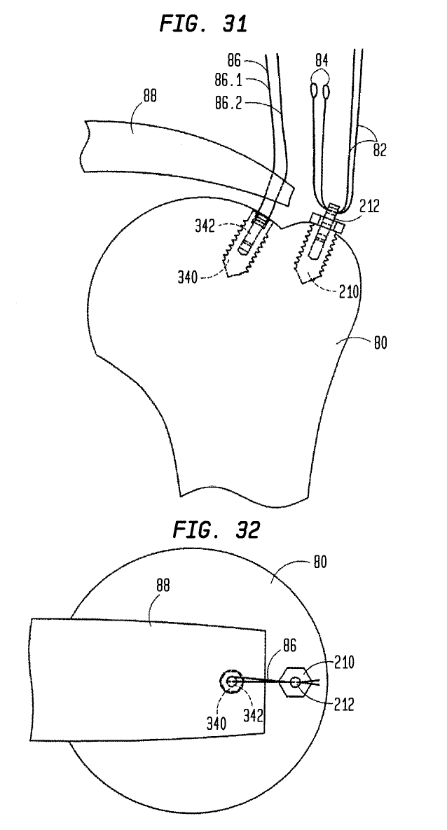

As a variation of the above sixth procedure, and as illustrated in FIGS. 31 and 32, for the location of the anchor main body 340 in the humerus, it is first displaced through the tendon 88 and then screwed into the humerus 80 in the location shown. By doing so the two segments 86.1 and 86.2 of the suture 86 are effectively passed through the tendon 88, as illustrated in FIG. 31. The remainder of the procedure is effectively the same as before, thus providing the anchored suture configuration as shown in FIG. 32.

Eighth Set of Exemplary Surgical Procedures

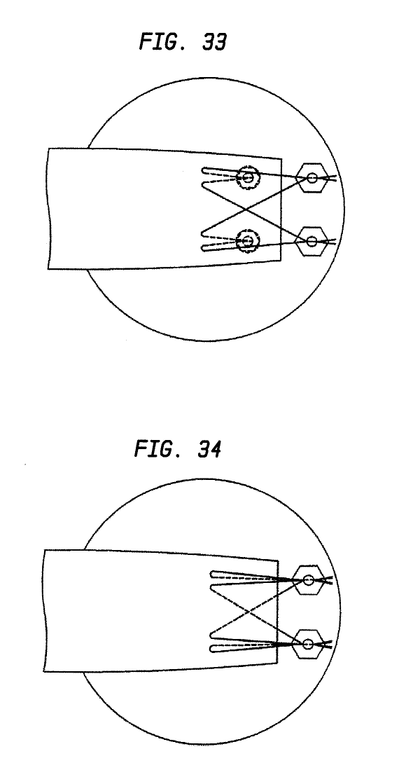

FIGS. 33 to 35 illustrate still further repair procedures in relation to the use of bone anchor devices as above described, FIG. 33 illustrating a procedure similar to that illustrated in FIGS. 29 and 30 except insofar as two pairs of bone anchor devices are used and one suture segment of the respective sutures crosses over as illustrated, in order to again create a more effective attachment footprint to provide for the secure attachment of a tendon to a humerus.

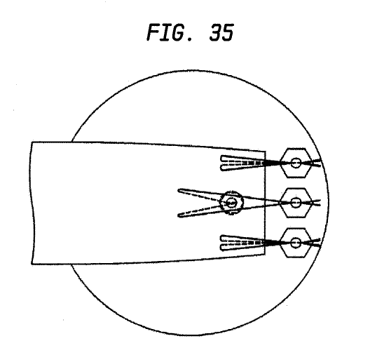

FIG. 34 also illustrates a cross-over procedure as above envisaged, but in relation to the procedure as illustrated in FIGS. 27 and 28, whereas FIG. 35 illustrates a procedure that involves a combination of the procedures described in FIGS. 27 and 28 and in FIGS. 29 and 30, as is clearly apparent. FIG. 35 illustrates a dual row fixation method, it being submitted that, in association with the repair of rotator cuff injuries, depending on the nature of individual injuries, particularly suitable repair procedures can be utilized in order to enhance and render most effective the repair of injuries. It will be appreciated that many further variations within the above procedures can be envisaged, a major benefit of the use of the procedures being that the need for suture knotting is completely eliminated, which will, in turn, significantly facilitate general suture management.

Fourth Set of Exemplary Embodiments

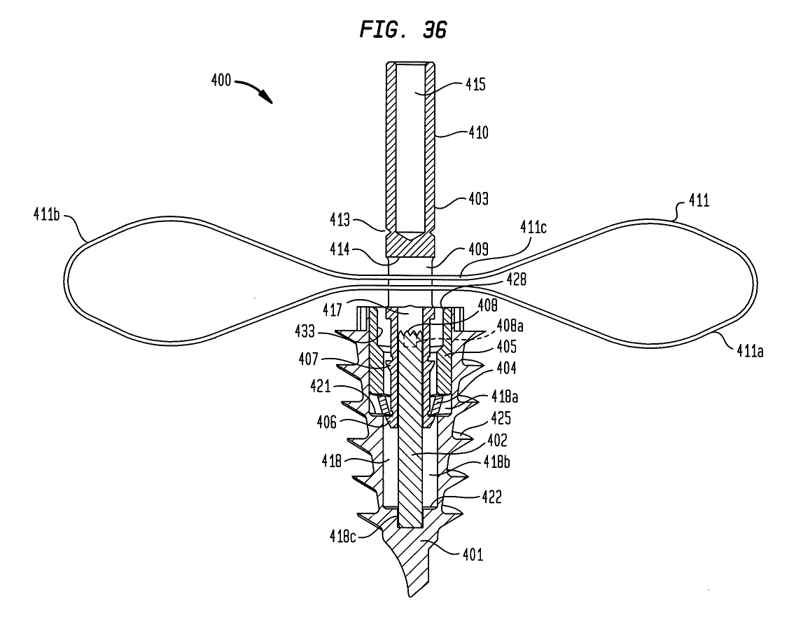

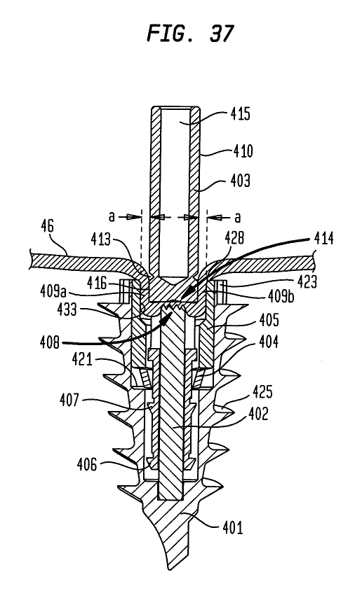

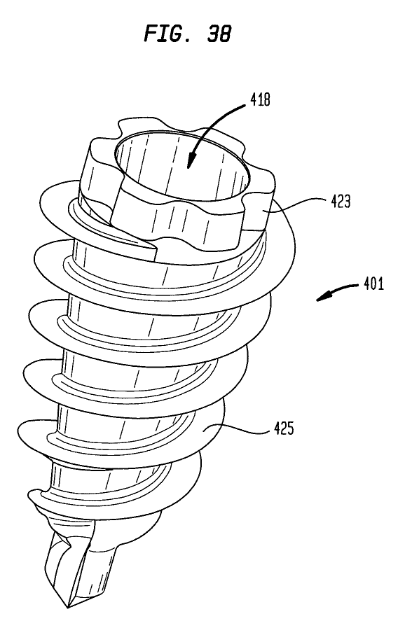

FIG. 36 is a cross-sectional side view of a bone anchor device 400 in the open state in accordance with a fourth embodiment of the present invention. FIG. 37 is a similar cross-sectional view, except showing the device 400 in the closed state. FIGS. 38-42 show some of the components of the overall bone anchor device 400 individually (i.e., disembodied from the overall device 400) for greater clarity.

Bone anchor device 400 in accordance with the fourth embodiment comprises a threaded anchor main body 401 (shown disembodied from the device in FIG. 38) in the nature of a screw or awl bearing threads 425, which can be screwed into a bone in a desired location as previously described. The anchor main body 401 comprises a central longitudinal bore 418 that is open at the proximal end and closed at the distal (or tip) end. The bore 418 comprises three segments, i.e., 418a at the proximal end, 418b in the intermediate portion, and 418c at the distal end. Segment 418a has the largest internal diameter, section 418b has an intermediate diameter, and segment 418c has the smallest diameter. The interface between segments 418a and 418b defines a first shoulder 421 and the interface between segments 418b and 418c defines a second shoulder 422. A shaped head 423 is provided at the proximal end of anchor main body 401 for engagement by a driving device such as a screwdriver or other geometrical driver, such as a Torx arrangement.

In other embodiments of this (or any of the other anchor main bodies described herein), the threads 425 on the anchor main body may be eliminated or reduced in size or replaced with ridges, striations, or other external formations and the bone anchor can be inserted into the bone by pounding (as in the nature of nail), instead of screwing. In such embodiments, a hole may be pre-drilled into which the anchor main body 401 is inserted.

A central pin 402 extends longitudinally in bore 418. The central pin 402 has a diameter slightly smaller than the diameter of distal bore segment 418c of anchor main body 401 such that it fits within segment 418c snugly but freely slidably therein in the longitudinal direction and freely rotatable about its longitudinal axis. In a preferred embodiment of the invention, the bore 418 and the pin 402 are cylindrical so that the pin 402 can rotate about its longitudinal axis relative to the anchor main body, which is a useful feature in many applications, as will be discussed in more detail below. However, in other embodiments, they may have non-cylindrical profiles since it is not required that the elements be rotatable relative to each other.

The proximal end 408 of the central pin 402 may be textured as shown to help grip sutures as will be discussed in more detail herein below. The texturing may take any number of forms. In one embodiment as illustrated, it comprises a series of peaks and valleys in the nature of an egg carton type shape. However, in other embodiments, the texturing may comprise parallel ridges, corrugations, serrations, divots, or general roughening of the surface. In yet another embodiment, a bore as shown in phantom at 408a in FIG. 36 may be formed in the central pin 402a.

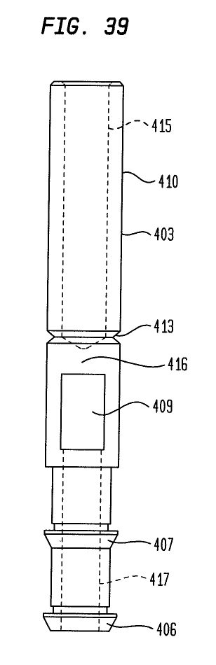

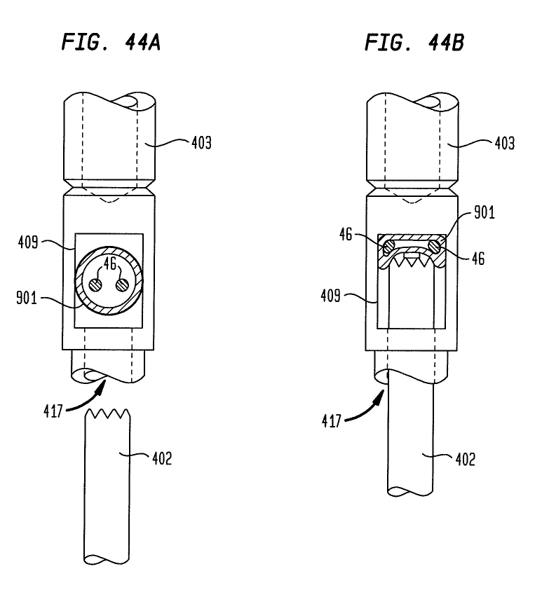

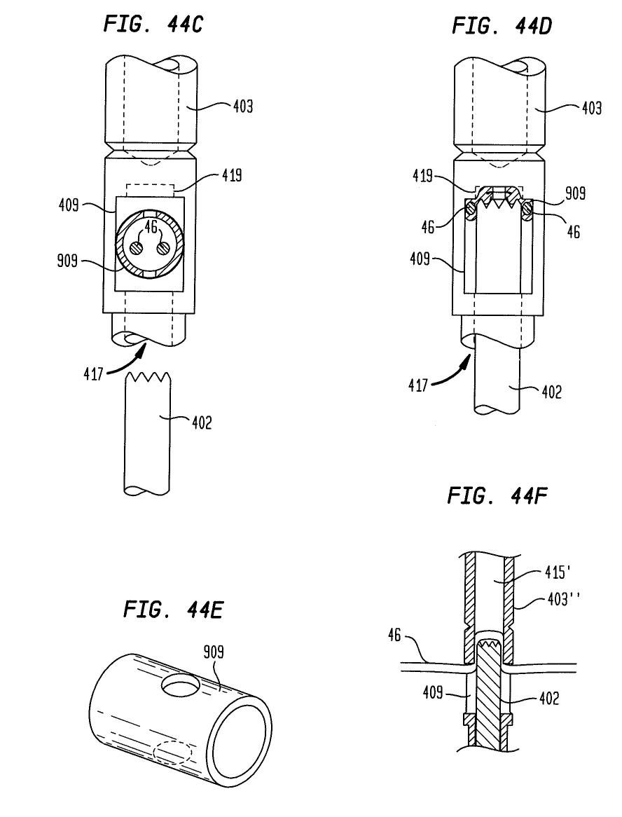

Next, an eyelet pin 403 (shown separately in FIG. 39) is disposed in the longitudinal bore 418 of the anchor main body 401 over the central pin 402. Particularly, eyelet pin 403 includes a transverse eyelet 409 intermediate its proximal and distal ends. One or more sutures will pass through the eyelet 409 and be locked in the device during the surgical procedure, as will be described in more detail herein below. Eyelet pin 403 includes a proximal bore 415 proximal of the eyelet 409 and a distal bore 417 distal of the eyelet 409. In the particular embodiment illustrated in FIGS. 36, 37, and 39, the proximal bore is blind to the eyelet 409, i.e., eyelet 409 and proximal bore 415 are not in communication with each other. However, as will be discussed below, in alternate embodiments, proximal bore 415 may extend completely through to and into communication with eyelet 409, e.g., as illustrated in FIG. 44F, discussed further below. The proximal longitudinal bore 415 is for the purpose of accepting a longitudinal member of an impactor tool as will be described in further detail herein below.

Distal bore 417 is open to and in communication with the eyelet 409. The diameter of distal bore 417 is equal to or slightly smaller than the diameter of central pin 402 so as to form an interference fit with the central pin, as will be described in more detail herein below. Thus, when assembled (in either the open position shown in FIG. 36 and the closed position shown in FIG. 37), the eyelet pin 403 and central pin 402 are not rotatable relative to each other, but the assembly of the eyelet pin and central pin collectively is freely rotatable relative to the anchor body because the central pin is freely rotatable in bore 418.

The distal portion of eyelet pin 403 includes two ramp formations 406 (near the distal end) and 407 (intermediate the distal end and the eyelet 409).

The proximal portion of the eyelet pin is a breakaway portion that will be removed from the body prior to the end of the surgery. The breakaway portion 410 is defined by a weakened section that can be broken relatively easily. This may be provided by a thinning of the material of the eyelet pin, such as by fabricating a radial notch or V-groove in the material, as illustrated at 413 in FIGS. 36 and 37.

The eyelet extension portion 410 serves several important functions. For instance, essentially the rest of the bone anchor device 400 other than extension 410 is embedded in and below the bone surface after installation of the bone anchor device in bone and, thus, is extremely difficult for the surgeon to see once installed, particularly in an arthroscopic procedure. However, the breakaway portion 410 of eyelet pin 403 protrudes substantially from the bone and is, therefore, easy to visualize. In one embodiment, at least the extension portion 410 of the eyelet pin 403 is brightly colored to even further enhance its visibility.

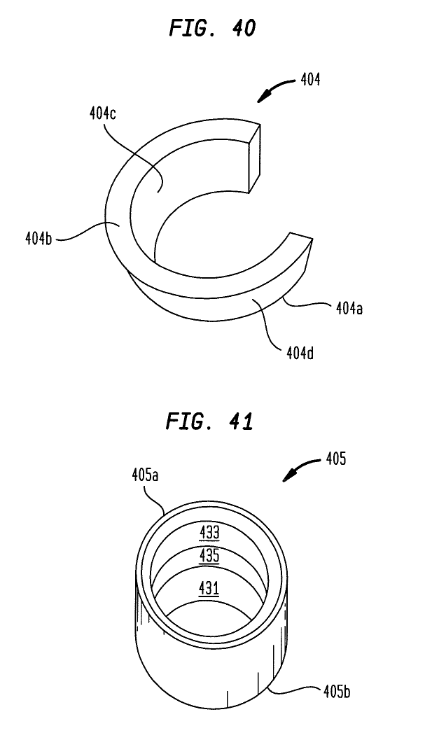

A locking ring helps retain the eyelet pin 403 in the anchor main body. In the embodiment shown in FIGS. 36 and 37, the locking ring 404 is a C-shaped ring (also shown separately in FIG. 40).

Locking ring 404 is made of a strong resilient material such as a metal or polymer so that, upon application of sufficient force in the radial direction, it can be spread radially outwardly, or squeezed radially inwardly, to change its diameter and return elastically when the force in the radial direction is removed. The inner and outer surfaces 404c, 404d of locking ring 404 are conical rather than cylindrical is shape. That is, inner and outer surfaces 404c, 404d are not parallel to the longitudinal axis 405 of locking ring 404 (i.e., up-down in FIGS. 26 and 37). Thus, a force applied to either surface 404c or 404d in the longitudinal direction (such as by ramp formations 406 or 407 on eyelet pin 403 hitting the inner surface 404c of locking ring 404 as eyelet pin 403 travels longitudinally in bore 418 of anchor main body 401) will be converted partially to force in the radial direction. Thus, if either ramp formation 406 or 407 meets the inner surface 404c of locking ring 404 with sufficient force, it can cause locking ring to radially expand outwardly, permitting that ramp formation to pass through the locking ring 404. When the force is removed, locking ring 404 returns elastically to its stress free (or unbiased) state.

Locking ring 404 is designed such that the required amount of force to make that happen is greater than could normally be applied accidentally, but that will permit ramp formations 406 and 407 to pass through locking ring by a moderate strike with a mallet on the proximal end of eyelet pin 404 during assembly or during surgery such, as will be described in further detail herein below.

An insert 405 is disposed in the proximal segment 418a of axial bore 418 in the anchor main body 401, as seen in FIGS. 36 and 37. The insert 405 also is shown separately in FIG. 41. Insert 405 is essentially a hollow cylinder having a constant outer diameter equal to or slightly larger than the inner diameter of proximal segment 418a or bore 418 in anchor main body 401, but comprising two sections 431 and 433 of different internal diameter. The distal section 431 has a narrower inner diameter than the proximal segment 433, thereby forming a shoulder 435 there between. Accordingly, insert 405 forms an interference fit within bore segment 418a essentially permanently fixing it in bore segment 418a in the position shown in FIGS. 36 and 37.

The inner diameter of the distal segment 431 of insert 405 is smaller than the largest external diameter of locking ring 404. The inner diameter of intermediate segment 418b of bore 418 in anchor main body 401 is smaller than the smallest outer diameter of locking ring 404. Accordingly, locking ring 404 is captured in segment 418a of bore 418 of anchor main body 401 between shoulder 421 between bore segments 418a and 418b and the distal end 405b of insert 405. The longitudinal length of insert 405 is selected so that, when insert 405 is fully inserted in bore 418 with its proximal end 405a essentially flush with the proximal end of anchor main body 401, the distance between the distal end 405b of insert 405 and shoulder 421 in axial bore 418 is slightly greater than the height of locking ring 404, thus essentially capturing locking ring 404 in the position as shown in FIGS. 36 and 37.

The bone anchor device 400 is assembled by first inserting the central pin 402 into bore 418 in the anchor main body 401. Particularly, it is inserted into the distal bore segment 418c of the anchor main body 401, as previously mentioned. Next, locking ring 404 is inserted into bore 418 where it will sit on shoulder 421. Next, insert 405 is press fit into proximal section 418a of bore 418, as previously described to capture locking ring 404 between insert 405 and shoulder 421.

Then, eyelet pin 403 is inserted into bore 418. Specifically, eyelet pin 403 falls readily through proximal bore segment 418a until it reaches central pin 402. whereupon it must be forced further downward over central pin 402 into an interference fit between the central pin 402 and the distal bore 415 of the eyelet pin 403, In addition, sometime after central pin 402 is in distal bore 415, ramp formation 406 comes into contact with the inner surface 404c of locking ring 404. Particularly, the largest diameter of ramp formation 406 is larger than the smallest diameter of the inner surface 404c of locking ring 404 when locking ring 404 is in its unbiased condition. Only upon application of significant downward force applied to ramp 406 on locking ring 404 will locking ring 403 be forced to expand radially sufficiently to permit ramp 406 to pass through.

Accordingly, sufficient force is applied downwardly on eyelet pin 403 to permit ramp formation 406 to pass through locking ring 404 (while simultaneously overcoming the continuing resistance to longitudinal movement of the eyelet pin 403 relative to the central pin 402 due to the aforementioned interference fit between the central pin 402 and the distal bore 415 of the eyelet pin 403. Once ramp 406 is through, the force is relieved and locking ring 404 returns to its stress-free state. At this point, the eyelet pin is now constrained in anchor main body 401 in the open position by virtue of first ramp formation 406 preventing the, now joined, eyelet pin 403 and central pin 402 from being pulled out proximally and the interference fit between central pin 402 and eyelet pin 403 preventing the joined eyelet pin 403 and central pin 402 from being pushed further into the bore 418 than the point at which the distal end of center pin 402 bottoms out in bore portion 418c. Accordingly, eyelet pin is axially trapped in anchor main body 401 with no or a very limited range of axial movement.

Only when sufficient downward force is again applied to eyelet pin 403 to (1) overcome the resistance to relative axial movement between the center pin 402 and the eyelet pin 403 resulting from the interference fit and (2) cause ramp formation 407 to expand locking ring sufficiently for ramp 407 to pass through locking ring 404 can eyelet pin 403 be disposed into the closed position as shown in FIG. 37.

The locking ring 404 illustrated in the Figures is exemplary. Other devices, particularly, other elastically deformable rings, can be substituted for the locking ring, such as an elastically deformable closed ring or a split ring (neither shown in the Figures). FIG. 49, for example, illustrates another ring structure 700 comprising four crescent elements 701 having grooves within which an O-ring 703 can be inserted into in a radial constraining arrangement. This arrangement 700 will operate essentially in the same manner as the above-described locking ring.

Exemplary Embodiments of a Driving Tool

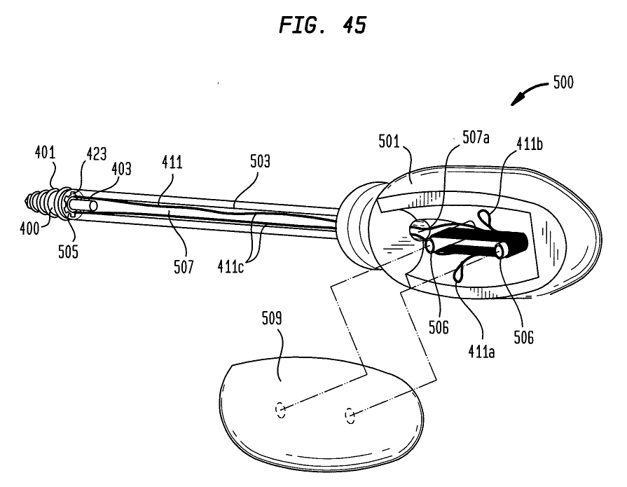

FIG. 45 shows a perspective view of an exemplary bone anchor driver tool 500. It comprises a cannula 503 defining an internal bore 507 and a handle 501 coupled to the proximal end of the cannula, with the proximal end 507a of the bore 507 being open to and in communication with the hollow interior of the handle 501.

As will be described in further detail immediately below, the ends of a suture shuttling mechanism, such as a wire or suture loop 411 or a long suture with a loop at each end threaded through the eyelet of the eyelet pin of a bone anchor device of the present invention, may run up the cannula 503 of the driver tool and extend into the hollow handle. The ends of the suture shuttling wire (or suture) may be wrapped around two pins 506 inside of the handle 503 for stowage and safe keeping prior to and during surgery. The handle can include a cap 509 to close off the handle if desired for better containment of sutures or suture shuttling mechanism 411, as will be described in detail further below. The bore is also open at recess 507b to the distal end of the cannula 507. The recess 507b at the distal end of the cannula is matingly shaped to engage the shaped head 423 of the anchor main body 401 of the bone anchor device so as to impart rotation to the anchor main body 401. As shown, when the driver 500 is engaged with the head of the anchor main body 401 of the assembled bone anchor device 400, the proximal end of the eyelet pin 403 extends within the cannula 507 of the driver 500. Preferably, the recess 507b is fashioned with gripping means, such as a slight interference fit over part of the mating surfaces of head 423 and recess 507b, so as to temporarily grip the head 423 of the anchor main body and hold it firmly so that the bone anchor device will not fall out of the driver unintentionally, but which can be released with moderate force once bone anchor 400 has been surgically located.

Ninth Set of Exemplary Surgical Procedures

The bone anchor device of FIGS. 36-41 can be used in surgical procedures for attaching soft tissue to bone such as those described herein above in connection with FIGS. 8-12, 13-15, and 16.

In fact, the various bone anchor and tissue fastener devices disclosed herein may be used in any number of surgical procedures, including those specifically described herein. In some such procedures, it may be desirable to provide a suture shuttle mechanism directly associated with the bone anchor device for shuttling sutures from the tissue fastener device or tissue (if no tissue fastener device is used) to the bone anchor device and, particularly, through the eyelet 409. In accordance with such embodiments, a shuttling mechanism comprising a flexible elongated member such as aforementioned wire loop 411 may be provided as shown in FIG. 36 passing through the eyelet 409. Wire loop 411 may be considered to comprise three segments, namely, opposing curved ends 411a and 411b, which are joined by linear segment 411c. Sutures may be inserted through one end of the loop, such as end 411b by a suitable instrument. The other end of the loop 411a may be pulled on to draw the loop 411, along with the shuttled sutures, through the eyelet 409. For instance, in one particular embodiment, the bone anchor device 400 is delivered to the surgeon already mounted on the driver tool 500. The loop 411 is long enough so that, with the center of the loop passing through the eyelet 409 of the eyelet pin 403 of the bone anchor device, both ends of the loop can extend up the entire length of the cannula 507 of the driver tool 500 and extending from the proximal end 507a of the cannula 507 into the handle 501, as shown in FIG. 45 illustrating the exemplary driver tool 500. Initially, the ends of the loop 411 may be wrapped around the two pins 506 for safe keeping within the interior of the handle. At the appropriate point in the surgical procedure, the wire ends can be unwrapped from the pins 506 so that both ends can be removed from inside the handle 503 of the tool 500 and may be manipulated manually by the surgeon externally of the patient. Having both ends of the loop extending from the driver tool provides several advantages. First, it can be used to shuttle sutures through the eyelet in either direction. Second, it helps prevent accidental deployment of one or both ends of the loop out of the instrument 500 and into the deployed position illustrated in FIG. 36. Particularly, if one or both ends of the loop 411 are disposed near the bottom of the tool 500, then a slight withdrawal of the tool from the bone anchor could release the end of the loop from the cannula. With both ends of the loop extending from the proximal end of the tool 500, this is much less likely. In addition, the surgeon can manually hold on to both ends, 411a and 411b, of the loop 411 in order to prevent one or both ends from being pulled through accidentally.

In any event, in an exemplary procedure, the surgeon would pull on one end of the loop, e.g., end 411a, until the other end 411b is released from the distal recess 507b of the cannula 507 of the tool 500 and into the deployed state. Then, the surgeon would thread the suture(s)-to-be-shuttled through the eyelet 409 of the bone anchor device through the deployed end 411b. After the sutures have been threaded through end 411b, the surgeon would merely need to grasp end 411a with his hand and pull so as to pull end 411b through the eyelet 409 and up through the cannula 507 until the end 411b of the loop 411 comes completely through the cannula 507, carrying the suture(s) with it. The surgeon can then disengage the suture(s) from the loop and manipulate the suture(s) directly, e.g., so as to pull the required tension on them before locking the eyelet in the closed position and cutting the free ends of the sutures.

The shuttling mechanism 411 may be made of thin, flexible wire. However, in alternate embodiments, it may be fabricated of any string or filament and, in fact, may be formed of suture itself. In an even further embodiment of the invention, the suture shuttle 411 need not be a closed loop. For example, the shuttling mechanism might be comprised of a length of suture folded in half, wherein the fold at the midpoint of the suture comprises the distal end 411b of the shuttling mechanism 411 and the two ends of the suture comprise the proximal end of the suture shuttle. To assist with shuttling, small loops may be formed in the ends of the suture (or other filament), such as illustrated by the suture shuttle shown in FIG. 45.

The bone anchor device 400, including the anchor main body 401, the central pin 402, the eyelet pin 403, the locking ring 404, and the insert 405, is delivered to the surgeon in the assembled, open state as shown in FIG. 36. During surgery, the surgeon will install the device 400 in bone by screwing it into a bone using a suitable driving device engaged with the head 423, such as driver tool 500 described herein above in connection with FIG. 45. Note that one of the beneficial features of the present invention is that, since the eyelet pin/central pin assembly is freely rotatable inside the anchor main body, there is relatively less need to worry about the rotational alignment of the anchor main body 401 when it is being screwed into the bone as compared to conventional suture anchors where the eyelet orientation is fixed. It can be screwed in to any rotational position because the eyelet pin 403 is freely rotatable therein to align the eyelet 409 to face in the desired direction (i.e., in the direction from which the sutures will enter the device 400).

Once installed, the surgeon will shuttle sutures through the eyelet of 409 in the eyelet pin 403 either using a shuttling mechanism such as the wire shuttling device 411 or another device so that one or more sutures pass through eyelet 409. Then, the surgeon will place an impactor tool into the proximal bore 415 in the extension portion 410 of eyelet pin 403. In an arthroscopic procedure, this would be done through a cannula. Then, while the surgeon is tensioning sutures acting on the tissue to locate the tissue in an appropriate anatomical position, sufficient force would be applied to the proximal end of the impactor tool, such as by hitting it with a mallet or using it in conjunction with a spring-loaded or pneumatic impacting device to pound the eyelet pin 403 with sufficient force to cause the second ramp formation 407 to spread apart locking ring 404 allowing it to pass through so that the eyelet pin 403 slides down over the central pin 402 into the closed position as shown in FIG. 37. Particularly, after ramp 407 passes locking ring 404, the interference fit between eyelet pin 403 and central pin 402 lock the two pieces 402, 403 together in the closed position.

As the eyelet pin 403 is driven down into the closed position, the suture(s) 46 passing through the eyelet at 409 gets trapped in at least one of three locations. First, as seen in FIG. 37, suture(s) may be crushed between the roof 414 of the eyelet 409 and the proximal end 408 of the central pin 402. Surgical sutures are highly compressible and deformable without breakage and the design of the interface between proximal end 408 of central pin 402 and roof 414 of the eyelet 409 accommodates varying suture diameters and numbers of sutures. Therefore, the length of central pin 402 should be selected relative to eyelet pin 403 so that the spacing between the roof 414 of eyelet 409 and the proximal end 408 of central pin 402, when in the closed position, is between zero and a full suture diameter, and preferably between about ⅛ and ¼ of a suture diameter wherein the locked, closed position. The features (e.g., roughening, peaks and valleys, serrations) at the proximal end 408 of the central pin 402 help better grip the sutures.

In addition, depending on the diameter of the central pin 402 relative to the cross section of the eyelet pin (i.e., the area in the direction transverse to the direction of the passage through the eyelet between its ends 409a and 409b), it is possible for sutures to become trapped between the radial circumferential surface of the central pin 402 and the side walls of the eyelet. These locations for trapping sutures 46 can be seen, for instance, in FIG. 44B, which will be discussed further below. Particularly, if the diameter of the central pin is smaller than the cross section of the eyelet 409 by less than the thickness of two sutures (and is centrally located in the eyelet in the direction transverse the passage and perpendicular to the longitudinal axis, i.e., in and out of the page in FIG. 37 or left and right in FIG. 44B), any sutures that do not become trapped between the proximal end 408 of the central pin 402 and the roof 414 of the eyelet 409 will be compressed and therefore, securely held between the side of the central pin and the side walls of the eyelet.

In addition, the suture(s) take on a tortuous shape, such as the W shaped illustrated in FIG. 37, thus providing even greater resistance to being pulled free of the bone anchor device 400.

In one embodiment of the invention, the features are small enough and deep enough so that they individually bore into the suture and split the fibers of the suture to provide an even stronger grip.

In addition, the suture is crushed between the surface 416 of eyelet pin 403 and the surface of the inner surface of the distal segment 433 of insert 405 at the transverse ends 409a, 409b of the eyelet 409. Specifically, the outer surface 416 of the eyelet pin 403 just above the eyelet 409 has a diameter relative to the inner diameter of the proximal segment 433 of insert 405 such that the clearance between the two surfaces is less than the width of the suture. The clearance preferably also may be somewhere between zero and ½ of the diameter of the suture, and more preferably somewhere between ⅛ and ¼ the diameter of the suture.

Note that the eyelet 409 need not even be completely within the receiving formation for there to be significant capturing of the suture. Specifically, even if the eyelet is only partially within the receiving formation in the longitudinal direction when in the closed position, the suture will be compressed between the roof 414 of the eyelet pin and the proximal end of the main anchor body as long as the distance (or clearance) between the roof 414 of the eyelet pin and the proximal end of the main anchor body in the longitudinal direction is less than a width of a suture (and those two surfaces are not too far from each other in the radial (or transverse) direction.

In alternate embodiments, the central pin 402 need not compress the suture against the roof of the eyelet at all, there being sufficient crushing and fixing of the suture in the other two locations in the lateral space between the inner diameter of the proximal portion 433 of the insert 405 and the surface 416 of eyelet pin 403.

In yet other embodiments, the roof 414 of the eyelet pin 403 may also be configured to help grip the suture. For instance, it may be provided with mating features to the features on the proximal end 408 of the central pin 402. Alternately, the roof 414 may have different features, such as roughening, serrations, corrugations, ridges, etc. In even further embodiments, the proximal end 408 of the central pin 402 and the roof 414 of the eyelet pin 402 may simply have mating shapes such as a V-shaped groove and a V-shaped protrusion or a ball and socket.

In yet other embodiments, a plug or insert may be affixed to the roof of the eyelet 409 to provide better gripping. Such a plug or insert may have some of the aforementioned features. In other embodiments, the insert may comprise a high friction material, such as silicone having a high frictional coefficient or any combinations of any of the above-noted features. It may also be fabricated from a dissimilar metal from the remainder of the eyelet pin 403. In yet other embodiments, it may comprise a rubber bumper or a leaf spring.

In a preferred embodiment of the invention, the proximal end of insert 405 is rounded over or flared, as shown by reference 428 so as to eliminate any sharp edges from contacting the suture and possibly causing it to tear or break.

Exemplary Embodiments of Impactor Tool

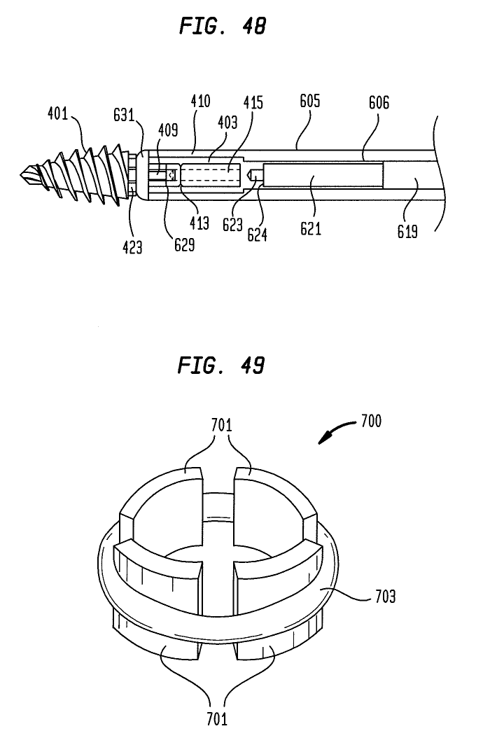

FIGS. 46-48 show an exemplary impactor tool 600 that can be used in connection with the bone anchor device 400 in the procedure described above. FIG. 46 shows the entire tool. FIG. 47 shows a close up view of the proximal portion of the tool. FIG. 48 shows a close up view of the distal portion of the tool. Tool 600 comprises an elongated tube 605 having an internal through bore 606. The opening 629 at the distal end of the tube (best seen in FIG. 48) is sized to snugly accept the eyelet pin 403 therein, but not the anchor main body 401, as shown. A handle 603 having a bore 613 coaxial with the bore 606 of tube 605 is mounted to the proximal end of tube 605. Disposed inside the handle and tube is a rod 619 that is spring loaded by a spring 611 constrained in handle 603. The spring has light force so as to keep the proximal end 607 of the rod 619 extending completely through the handle 603 so that the proximal end 607 of is exposed such that it can be hit with a mallet or other impacting device. A block 615 is fixedly attached to the rod 619 near the proximal end 607, but trapped within the handle 603. Block 615 provides a stop for the spring 611, which is trapped between the block 615 and the distal end 617 of the handle 603. The spring 611 and block 615, when unbiased, maintain rod 619 in the shown position. Thus, striking end 607 of rod 619 drives the rod 619 down through the handle 603 and tube 605. Although not shown, an enlarged, more stable striking surface for the mallet may be provided either integral with proximal end 607 of rod 619 or as a separate piece that slidably fits over proximal end 607 of rod 619. The enlarged striking surface may be metal, plastic, or any other suitable material.

The distal end of the rod 619, as best seen in FIG. 48, includes a narrowed diameter portion 621 and an even smaller diameter portion (or pin) 623 at the distal end. Portions 623 and 621 are designed so that pin 623 will slidably but snugly fit within the proximal bore 415 of the eyelet pin 403 and the shoulder 624 between pin 623 and narrowed portion 621 will butt up against the proximal end of the eyelet pin 403 when spring 611 is sufficiently compressed. However, in the unbiased condition, as shown in FIGS. 46-48, pin 623 is not engaged in proximal bore 410 of eyelet pin 403, but is coaxial with but slightly spaced from bore 410. The aforementioned spring 611 maintains the rod in this spaced position from the bone anchor device. A bumper (or ring) 631, comprised, for instance, of silicone, is attached to the distal end of tube 605 having a hole 632 aligned coaxially with hole 629 in the end of tube 605. However, in other embodiments, pin 623 may be disposed in bore 410 with the shoulder 624 resting against the proximal end of the eyelet pin 403.

In operation, when it is time to drive the eyelet pin 403 from the open position illustrated in FIG. 36 to the closed position illustrated in FIG. 37, impactor tool 600 is slipped over the bone anchor device 400 as shown in FIG. 48. Particularly, bumper 631 is slid over the extension portion 410 of the eyelet pin 403 until it butts up against the head 423 of the anchor main body 401 of the bone anchor device 400. Any sutures (not shown in FIG. 48) passing through eyelet 409 in eyelet pin 403 would be temporarily held between the head 403 of the anchor main body 400 and the bottom of the bumper 631. Since the bumper is soft, the sutures would be able to slide, upon being pulled by the surgeon between the head 423 and the bumper 631.

In use, after positioning the impactor tool over the eyelet pin extension portion 410 as shown in FIG. 48, the surgeon will grab the end of the suture or sutures through another cannula and pull to the desired tension, drawing the tissue into the desired position relative to the bone. The surgeon can then push the impactor tool 600 down on the top of the anchor main body 401 with some additional force, to hold the sutures in this tensioned state between the bottom of the bumper 631 and the top of the anchor main body 401. The surgeon can then let go of the sutures and the interaction between the bumper and the top of the anchor main body 403 will hold the sutures in this tensioned position, without damaging the sutures, until the surgeon can strike the impactor tool 600, causing the eyelet pin 403 to be driven downwardly into the closed position in which the sutures will be locked in the bone anchor device 400.

Specifically, when the surgeon strikes the proximal end 607 of the impactor tool 600, pin 623 descends into bore 415 and drives eyelet pin 403 down into anchor main body 401 to the closed position shown in FIG. 37. Particularly, the force of the impact being sufficient to force the second ramp formation 407 through locking ring 404 and to overcome the interference fit between central pin 402 and eyelet pin and distal bore 418 of eyelet pin 403). When ramp formation 407 passes distal surface 404a of locking ring 404, locking ring 404 returns elastically to its stress-free state against shaft 419 of eyelet pin 403.

Preferably, the diameter of the pin 623 is slightly larger than the diameter of the proximal bore 415 of the eyelet pin such that the pin 623 forms an interference fit inside the bore 415 at this time. Preferably, the interference fit is relatively weak so that the eyelet pin 403 can be removed from the impactor tool 600 at a later time.

When the eyelet pin 403 is in the open position, the V-groove 413 defining the breakaway portion 410 of the eyelet pin is preferably proximal to the bumper 631, as shown in FIG. 48. Accordingly, the soft bumper 631 and distal tip of cannula 605 helps unload the force of the impact from the V-groove 413 so as to help prevent it from accidentally breaking prematurely before or during impact.

After the eyelet pin 403 is driven down into the closed position, the impactor tool 600 is then used to break off the breakaway portion 410 of the eyelet pin 403. This is achieved by rocking the impactor tool (and the cannula within which it is inserted in an arthroscopic procedure) back and forth so that it pivots about the bumper 631 engaged with the top of the anchor main body 401. Particularly, when eyelet pin 403 is in the closed position, the V-groove 413 in the eyelet pin 403 is essentially even with the top of the anchor main body 401, and thus with the bottom of the bumper 631. The bumper permits the impactor tool 600 to be rocked back and forth so that the V-groove can be broken without metal to metal contact between the impactor tool 600 and the anchor main body 400. Once broken, the breakaway portion of the eyelet pin will stay inside the impactor tool because of the weak interference fit between the pin 623 at the end of the rod 619 of the impactor tool 600 and the proximal bore 415 of the eyelet pin. Alternately or additionally, the hole 632 defined by the ring-shaped bumper may be designed to be slightly smaller than the diameter of the extension portion 410 of the eyelet pin so that the bumper must slightly deform radially outwardly when it is slipped over the extension 410 providing a tight, but still slidable fit with the extension 410. This would provide an alternative or additional means of retaining the breakaway portion 410 of eyelet pin 403 inside the impactor tool 600. The impactor tool 600 can then be removed with the breakaway portion 410 contained therein.

In other envisioned embodiments of the invention, a tool that is capable of delivering a precisely controlled striking force may be used instead of a simple mallet. The tool would be adapted to fit over the proximal end 607 of the rod 619 and to deliver a blow along the longitudinal axis of the rod 619. For instance, Applicants envision a spring-loaded tool, wherein the spring loading is released by a small tap of a mallet, the spring selected and pre-loaded to deliver the exact amount of force desired over the exact travel distance desired. This force should be sufficient to push ramp formation 406 or 407 through locking ring 404 as previously described, but not so much as to injure the bone. In other embodiments, the spring may be released by a trigger mechanism instead of a mallet.

Fifth Set of Exemplary Embodiments

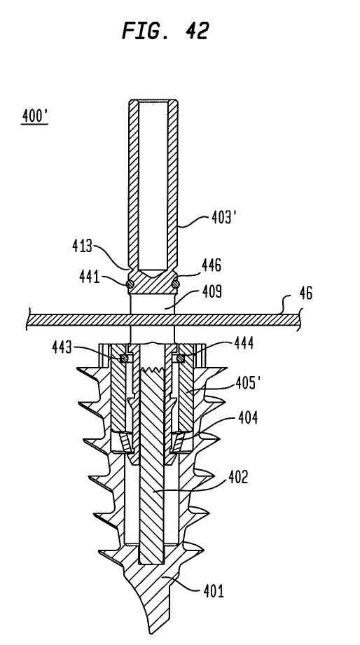

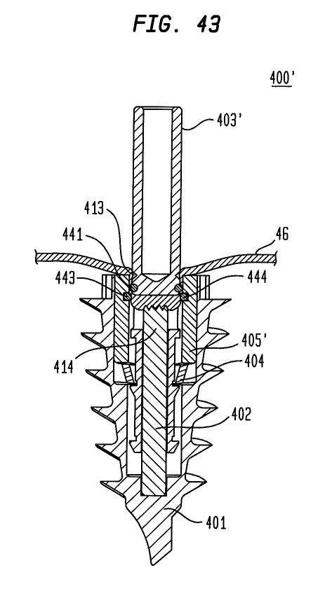

FIGS. 42 and 43 are cross-sectional views illustrating an alternative embodiment 400′ to the bone anchor device 400 shown in FIGS. 36-41. FIG. 42 shows the bone anchor device 400′ in the open position, while FIG. 43 shows it in the closed position. The device 400′ is largely similar to device 400 shown in FIGS. 36-41. However, it includes two O-rings 443 and 441 that assist with suture management. Particularly, in this embodiment, the insert 405′ is slightly modified from the insert 405 of FIGS. 36, 37 and 41. Particularly, it includes a groove 444 near its proximal end 405′ within which a silicone or other resilient material O-ring 443 sits. In a similar manner, eyelet pin 403′ also is adapted to have another groove 446 for accepting another O-ring 441 positioned just above the eyelet 409. As can be seen in FIG. 43, when in the closed position, O-rings 441 and 443 meet and press against each other near the top of the anchor main body 401, precisely where the suture 46 passes through the bone anchor device 400a. The soft material of the O-rings 441 and 443 grips the suture tightly and also prevents the suture from contacting metal at this juncture, thereby helping assure that the sutures are not damaged or broken during or after the eyelet pin is driven into the closed position. The O-rings may be formed of high friction silicone or any other reasonably resilient material.

In yet other embodiments of the invention, other features similar in shape and position to the O-rings 441 and 443 may be provided. Those features may be formed of materials other than the material of the eyelet pin 403 and/or insert 405. Alternately, the features may be formed directly into the eyelet pin 403′ and/or insert 405′. The features should have rounded non-sharp shapes that help grip the suture without damaging it.

Sixth Set of Exemplary Embodiments

FIGS. 44A-44F illustrate further embodiments of the invention. For sake of clarity, only the eyelet pin 403 and the central pin 402 are shown in each of FIGS. 44A-44E. However, it should be understood that these components are disposed in the anchor main body 401 with the other elements, such as locking ring 404 and insert 405, but they are not shown in these Figures in order not to obfuscate the features being particularly illustrated in these Figures. The angle of view in FIGS. 44A-44D is rotated 90° from the angle of view in FIGS. 36 and 37.

FIGS. 44A and 44B illustrate a first alternate embodiment of the bone anchor device 400 in which a hollow cylinder 901 is disposed in the eyelet 409. The hollow cylinder 901 is formed of a thin-walled deformable material, such as metal. In one embodiment, the material is plastically deformable. However, if it also could be elastically deformable. In the illustrated embodiment, the hollow cylinder 901 is circular and the eyelet 409 is square with the hollow cylinder 901 sized to have a diameter equal to the transverse cross-section of the eyelet 409. Therefore, the hollow cylinder 901 contacts the sides of the eyelet at two locations spaced 180° around the hollow cylinder 901. However, in other embodiments, the eyelet could be square so as to contact the eyelet at four locations spaced at 90° intervals around the hollow cylinder. According to even further embodiments, the hollow cylinder could be oval (and may or may not contact the eyelet at four locations spaced at 90° intervals around the hollow cylinder).

The sutures 46 that pass through the eyelet 409 pass through the middle of the hollow cylinder 901.

Referring now to FIG. 44B, which shows the condition of the components when in the closed position, when the eyelet pin 403 is driven down so that central pin 402 enters the eyelet 409 as previously described, it impinges upon the hollow cylinder 901, deforming it into the shape shown in FIG. 44B. As can be seen, the eyelet 409, hollow cylinder 901, and central pin 402 are sized relative to each other such that the sutures 46 are crushed by the hollow cylinder 901. In other words, the clearance between the central pin 402 and the sides of the eyelet 409 is less than the diameter of the suture such that the suture gets fixedly trapped or compressed. One or more sutures also may get fixedly trapped in between the proximal end 408 of the central pin 402 and the roof 414 of the eyelet 409.

This configuration may provide stronger gripping of the sutures.

FIGS. 44C, 44D, and 44E illustrate another alternate embodiment involving a modified cylinder 909. FIG. 44C shows this configuration in the open state and FIG. 44D shows it in the closed state. FIG. 44E shows a perspective view of the cylinder 909 disembodied from the device for sake of clarity. These Figures illustrate two alternate features relative to the device shown in FIGS. 44A and 44B that can be incorporated individually or in combination into the device.

First, ring 909 has a hole 911 and optionally a second hole 912 formed therein coaxial with each other, and the ring 909 is inserted into the eyelet with the holes coaxially aligned with the distal bore 417 of the eyelet pin 403. Second, an opening 419 through which the central pin 402 can pass may exist in the roof or top wall 414 of the eyelet 409. Alternately, the proximal bore 415 may simply extend all the way to and in communication with the eyelet, thereby providing the opening in the top wall 414 of the eyelet. The holes 911, 912 are smaller than the central pin 402 such that the central pin cannot pass through eyelet without also deforming the holes 911, 912 as well as the ring 909 itself.

As shown in FIG. 44D, in this embodiment, when the central pin 402 is driven through the eyelet 409, it punches through the bottom hole 911, thereby deforming the cylinder 909 as shown and capturing the sutures inside the crushed ring 909. In addition, if an opening 419 is provided in the top wall 414 of the eyelet and/or a second hole 912 is provided in the ring 909, the central pin may punch through the top hole 912 and/or the opening 419. As in the embodiment of FIGS. 44A and 44B, the sutures become fixedly trapped above the proximal end 408 of the central pin in the ring 909 and/or in opening 419. In the embodiment of FIGS. 44C and 44D, at least those sutures that are located in opening 419 of the eyelet pin 403 take on an even more tortuous path, thereby providing even greater gripping of the sutures in the bone anchor device.

FIG. 44F shows an even further embodiment of the invention in which the proximal bore 415′ of the eyelet pin 403″ extends completely through and is in communication with the eyelet 409 such that there is a bore running continuously through the eyelet pin from the distal end, through the eyelet, and to the proximal end of the eyelet pin 403″. In this embodiment, there is no surface in the roof of the eyelet 409 that the proximal end 408 of the central pin 402 can crush sutures up against. Nevertheless, sutures that do end up above the central pin 402, rather than on the sides thereof, take on a particularly tortuous path, and therefore are still tightly gripped in the bone anchor device.

The various different hollow cylinders 901, 909 and the various different configurations of the bore 415 and 417 in the eyelet pin 403 can be combined with each other in various permutations. For example the hollow cylinder 901 need not be a continuous ring and may have a circumferential gap (e.g., a split hollow cylinder) such as a rolled piece of thin metal or a roll pin.

In other embodiments, as already noted, the hollow cylinder need not be perfectly cylindrical, but can have an oblong or oval cross-section. In such embodiments, the eyelet can be rectangular so as to match the dimensions of an oval hollow cylinder (i.e., contacting it at four locations spaced 90° from each other around the circumference of the hollow cylinder) or it can have a square profile such that the hollow cylinder only contacts the eyelet at two location spaced 180° from each other around the circumference of the hollow cylinder.

In any of the embodiments discussed hereinabove in connection with the use of a hollow cylinder in the eyelet, it may be preferable to round out the proximal end of central pin 402 so as to avoid any sharp edges. This would help avoid the possibility of the central pain punching a hole through the hollow cylinder without substantially deforming it.

Seventh Set of Exemplary Embodiments

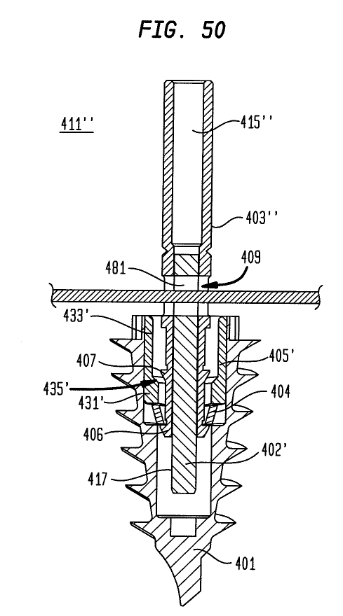

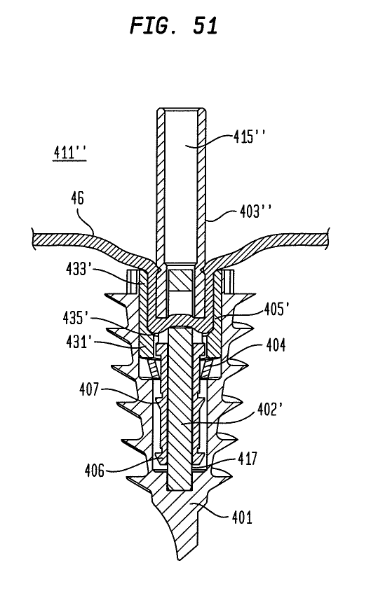

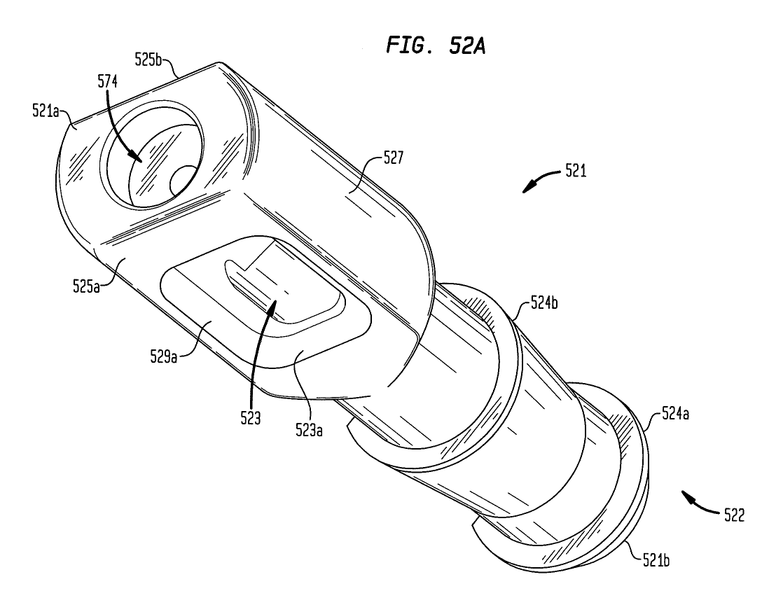

FIGS. 50 and 51 are cross-sectional views illustrating another alternative embodiment 400″ to the bone anchor device 400 shown in FIGS. 36-41. FIG. 50 shows the bone anchor device 400″ in the open position, while FIG. 51 shows it in the closed position. The device 400″ is largely similar to device 400 shown in FIGS. 36-41. However, the eyelet pin 403″, insert 405′, and central pin 402′ are modified, providing a different mechanism for fixing a suture 46 in the bone anchor device 400″. Particularly, the significant modifications are as follows. First, central pin 402′ includes its own eyelet 481 near its proximal end, which aligns with eyelet 409 in the eyelet pin 403″ when in the open position, as shown in FIG. 50. Second, the proximal bore 415″ in the eyelet pin 403″ is slightly larger in diameter than the distal bore 417 and the central pin 402′. However, it should be noted that this is not necessarily a modification since the diameter of the proximal bore 415 relative to the distal bore 417 in the embodiment of FIGS. 36-41 was not specified. Also, the proximal bore 415″ extends to and is in communication with eyelet 409 in the eyelet pin 403 (similarly to the embodiment of FIG. 44F). Insert 405 also is modified such that the shoulder 435′ between the larger internal diameter of the proximal segment 433′ and the smaller internal diameter of the distal segment 431′ is lower. Although, again, this is not necessarily a modification since the position of shoulder 435 between the larger internal diameter of the proximal segment 433 and the smaller internal diameter of the distal segment 431 of the insert 405 in the embodiment of FIGS. 36-41 was not specified.

In this embodiment, the suture becomes locked in the device 400″ by means of the two eyelets 409 and 481 shifting in longitudinal position relative to each other. Particularly, in the open position, the eyelet 481 in the central pin in longitudinally aligned (and also rotationally aligned about the longitudinal axis) with the eyelet 409 in the eyelet pin so that one or more sutures may pass through the eyelets 409, 481 essentially as described in connection with the embodiment of FIGS. 36-41. Then, when the eyelet pin is driven downwardly, the central pin moves downwardly until it bottoms out in the bottom of distal segment 418c of bore 418 in anchor main body 401, whereupon the force imparted to eyelet pin 403″ overcomes the force of the interference fit between the central pin 402′ and the distal bore 417 of eyelet pin 403″ as well as forces ramp formation 407 past locking ring 404 and into the closed position. This causes the eyelet 409 in the eyelet pin 403″ to move downwardly relative to the eyelet 481 in the central pin 402′. It can be seen in FIG. 51 that, in the closed position, the resulting longitudinal misalignment of the two eyelets 481 and 409 causes any suture(s) passing through the eyelets to take on a tortuous path and to become compressed and locked to the bone anchor device 400″ at four separate locations. The first two are two of the same locations as in the embodiment of FIGS. 36-41, namely, at opposite ends 409a and 409b of the eyelet 409 between the outer surface 416 of the eyelet pin and the proximal section 433′ of the insert 405′. The other two are between the surface of the central pin 402′ and the proximal bore 415″ of the eyelet pin 402″, as indicated at 463 in FIG. 51.

It now should be apparent that the reason the proximal bore 415″ is preferably slightly larger than the distal bore 417 proximal bore and the central pin 402′ is to provide clearance for the sutures between the two. It also should now be apparent that the reason the shoulder 435′ in the insert preferably is lower than in the embodiment of FIGS. 36-41 also is to provide sufficient clearance for the suture(s) between the insert inner bore and the surface 416 of the eyelet pin 416. More particularly, in this embodiment, because there must be room in the portion of the eyelet pin 403″ above the eyelet 409 to accommodate both the eyelet 481 of the central pin 402′ and a portion of the pin 402′ above the eyelet 481 while still preferably maintaining the breakaway V-groove 413 essentially flush with the top of the anchor main body 401 in the closed position, the eyelet 409 in the eyelet pin 403″ preferably is positioned lower into the anchor main body 401 when in the closed position than in the embodiment of FIGS. 36-41. Of course, these particular modifications are merely exemplary insofar as different sets of modifications may be implemented to achieve similar goals.

This embodiment provides secure fixing of the suture(s) in the bone anchor device

In these types of embodiments, the bone anchor device could even possibly be delivered to the surgeon already in the closed state with or without one or more sutures already disposed in and passing through the eyelet.

Eighth Set of Exemplary Embodiments

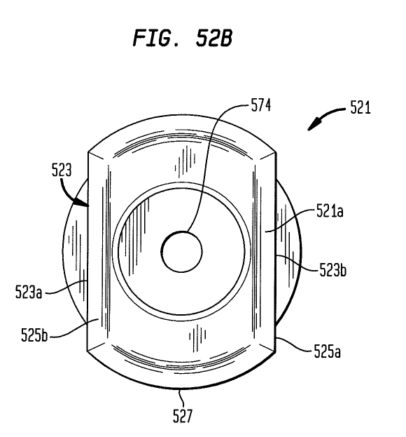

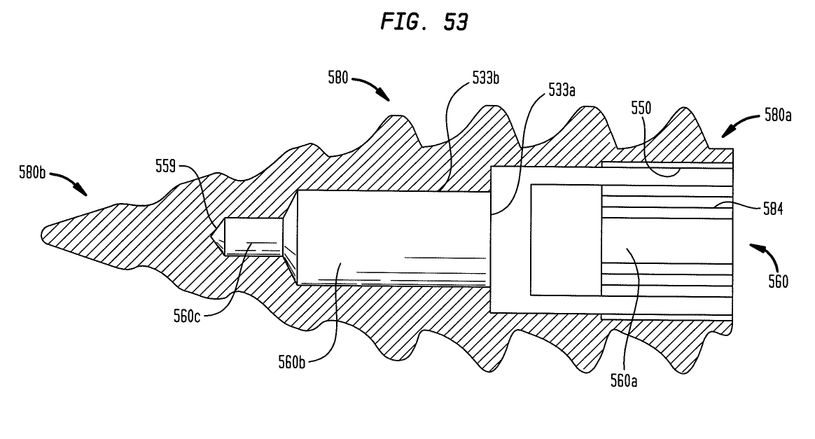

FIGS. 52A-56C illustrate another set of embodiments of a bone anchor in accordance with the present invention as well as a tool for implanting the bone anchor. Some of the benefits of these embodiments include that the bone anchor may be implanted using a single tool, the bone anchor and tool can be delivered to the surgeon as a single inseparable unit until implantation so that no parts can be lost and there is no possibility of incorrect assembly. It also facilitates ease of use.

The bone anchor in this embodiment is similar in many respects to the bone anchor embodiments disclosed in FIGS. 36-41. Accordingly, the following discussion will focus primarily on the differences of this embodiment relative to the embodiments of FIGS. 36-41.

An example of an eyelet pin in accordance with this set of embodiments is shown in perspective view in FIG. 52A and in plan view in FIG. 52B. A cross-sectional side view of an example of an anchor main body in accordance with this set of embodiments is shown in FIG. 53. In this embodiment, the eyelet pin 521 is modified in several respects. First, the cylindrical radial surface of the eyelet pin 410 of FIGS. 36-41 is modified in the present embodiment to have two flattened portions 525a, 525b near its proximal end adjacent the opposite ends of the eyelet 523, as best seen in FIG. 52B. This feature provides several significant benefits. First, it allows the clearance between the outer radial wall 527 of the eyelet pin 521 and the inner radial wall 550 of the anchor main body 580 (see FIG. 53) to be much smaller in all places except at the flattened portions 525a, 525b. More particularly, as previously noted, a clearance must be provided between the eyelet pin outer radial wall and the main anchor body inner radial wall for the sutures that will pass through the eyelet 523 and out of the anchor main body 580. However, this clearance is necessary only at the opposite ends of the eyelet 523 and nowhere else. Thus, by flattening the radial side wall 527 of the eyelet pin 521 adjacent the opposite ends of the eyelet 523, the eyelet pin diameter (in the portions of the eyelet pin that are still cylindrical) can be made larger so as to minimize the clearance between the eyelet pin 521 and the anchor main body 580 at the proximal end 521a of the eyelet pin 521. This allows the eyelet pin to be made sturdier because it is thicker (except in the portions where it is flattened).

The flattened portions 525a, 525b also provide a benefit with respect to the fabrication of the eyelet pin 521. Particularly, the flattened portions 525a, 525b make it easier to form bevels 529 at the opposite ends of the eyelet 523 and to eliminate sharp edges where the sutures will enter the eyelet. Sharp edges at the opposite ends of the eyelet could obstruct effortless passage of sutures through the eyelet. The beveled edges provide a funnel-like entry to the eyelet, thus gathering the suture bundle prior to entering the eyelet. If the outer radial surface of the eyelet pin were curved at the opposite ends of the eyelet, it would be more difficult to machine or otherwise form the bevels 529 without also forming sharp edges in the bevels themselves.

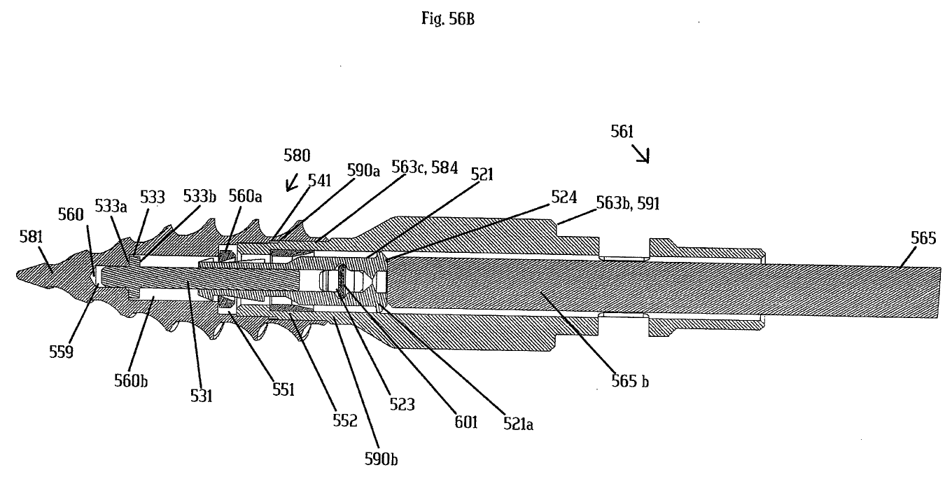

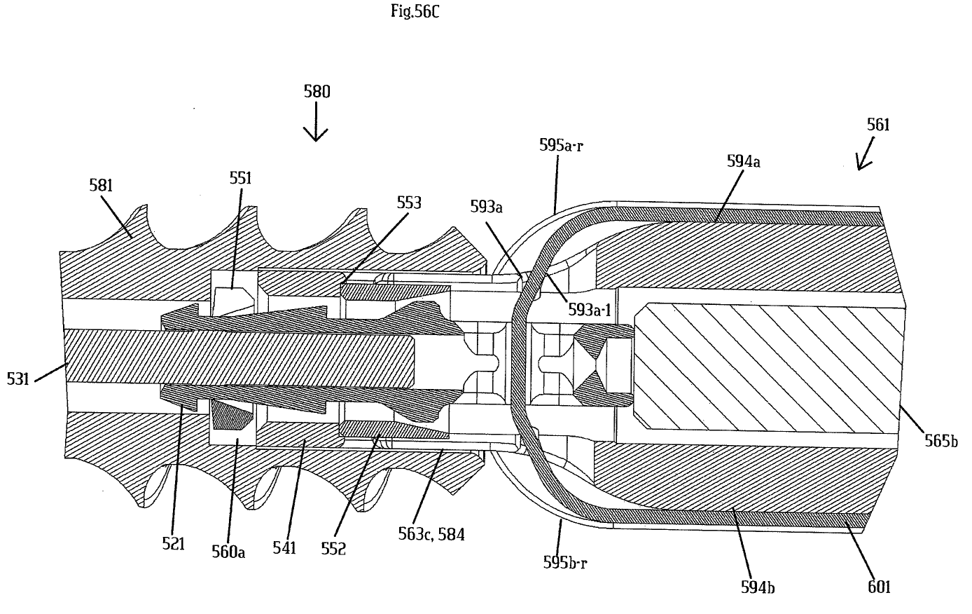

Also in this embodiment and with reference to FIGS. 56A-56C, which are an exploded view and two orthogonal side cross-sectional assembled view, respectively, of the bone anchor and associated implantation tool, the central pin 531 has a shelf 533 formed therein. The distal surface 533b of the shelf serves as a stop for the central pin 531 from having its distal end bottom out on the distal extent 559 of the longitudinal bore 560 of the main anchor body 580, while the proximal surface 533a defines a stop for the eyelet pin 521 relative to the central pin 531. Particularly, as will be discussed in more detail below, the distal end 521b of the eyelet pin 521 will hit and be stopped by the upper surface 533a of the shelf 533 as the eyelet pin 521 is being driven down over the central pin 531 from the open condition to the closed condition.

In this embodiment, the breakaway portion 410 of the eyelet pin 401 of FIGS. 36-41 is eliminated. Since, as will be discussed in more detail below, the anchor and tool are attached to each other until near the end of the implantation procedure, there is no need for an eyelet pin extension to help with finding the bone anchor after it is implanted.

With reference to FIG. 53, the anchor main body 580 in this embodiment also differs slightly from the anchor main body 401 in the embodiments of FIGS. 36-41. As in the embodiments of FIGS. 36-41, the anchor main body 580 has an internal bore 560 having three different sections 560a, 560b, 560c of decreasing diameter from proximal end 580a to distal end 580b. As best seen in FIG. 56A, the bore 560 accepts the central pin 531, eyelet pin 521, C-ring 551 and retaining ring 541 as in the embodiments of FIGS. 36-41. However, in the embodiments of FIGS. 36-41, the proximal end of the anchor main body 401 has an external formation 423 for mating with a torquing tool having mating internal formations for screwing it into bone. In this embodiment, on the other hand, the proximal end 560a of the longitudinal internal bore 560 of the anchor main body 580 bears a formation 584 for mating with external formations on a torquing tool (not shown) to permit the torquing tool to rotate the bone anchor for purposes of screwing the bone anchor into bone.

Finally, with reference to FIGS. 56A-56C, the retaining ring 541 that holds the C-ring 551 (or other locking ring or mechanism for maintaining the eyelet pin in the anchor) differs from the insert 405 shown in the embodiments of FIGS. 36-41. In accordance with the present embodiment, the retaining ring 541 per se may be virtually physically identical to the insert 405 discussed above in connection with the embodiments of FIGS. 36-41, except that the retaining ring 541 is integral with the implantation tool in the pre-surgical condition. The retaining ring 541 becomes separated from the implantation tool only towards the end of the implantation process, as will be described in more detail below.

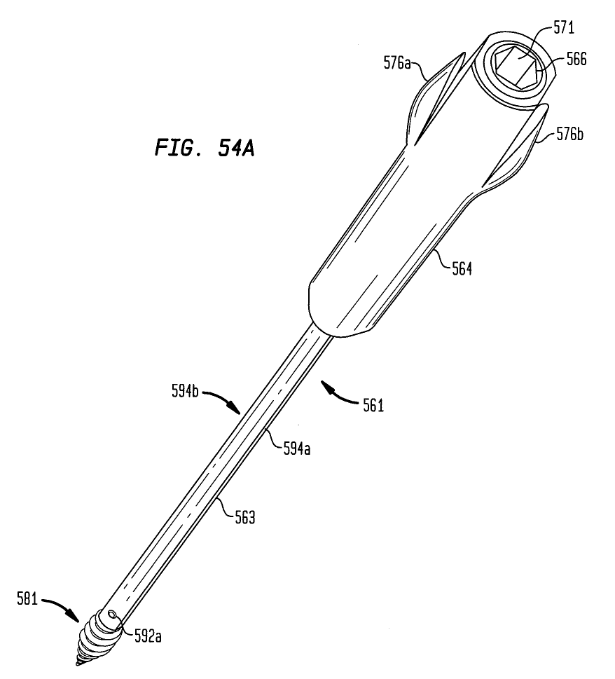

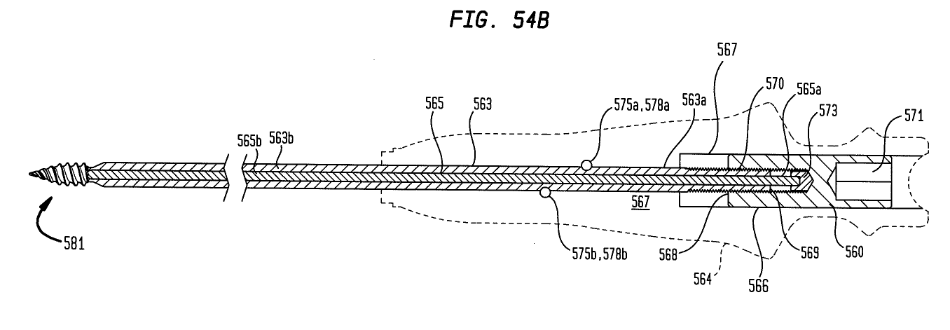

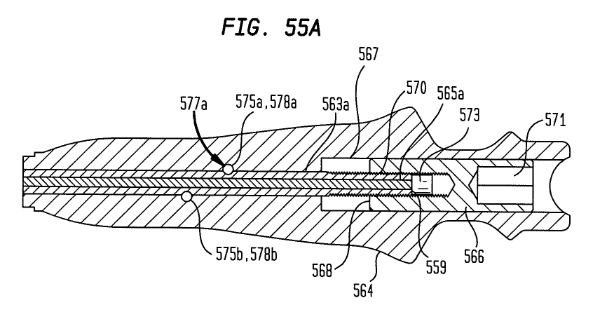

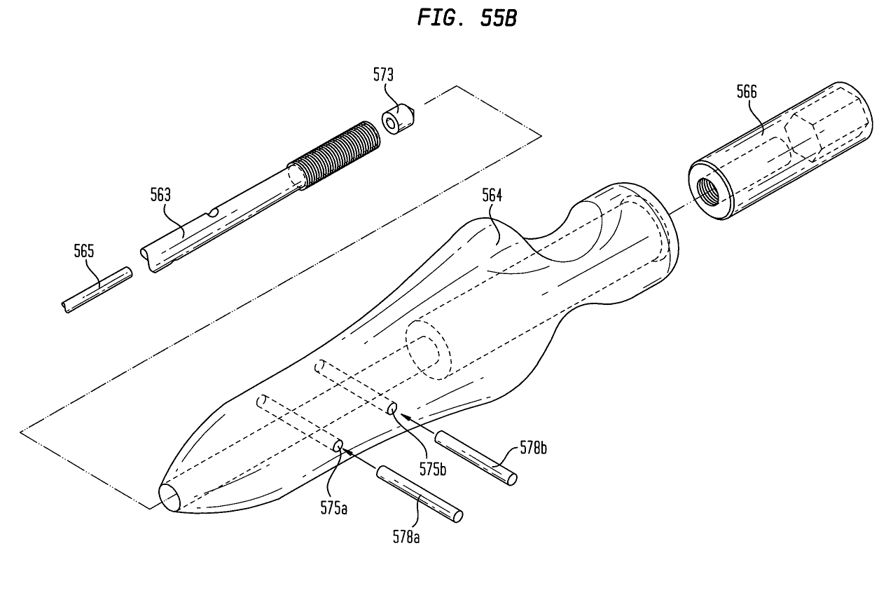

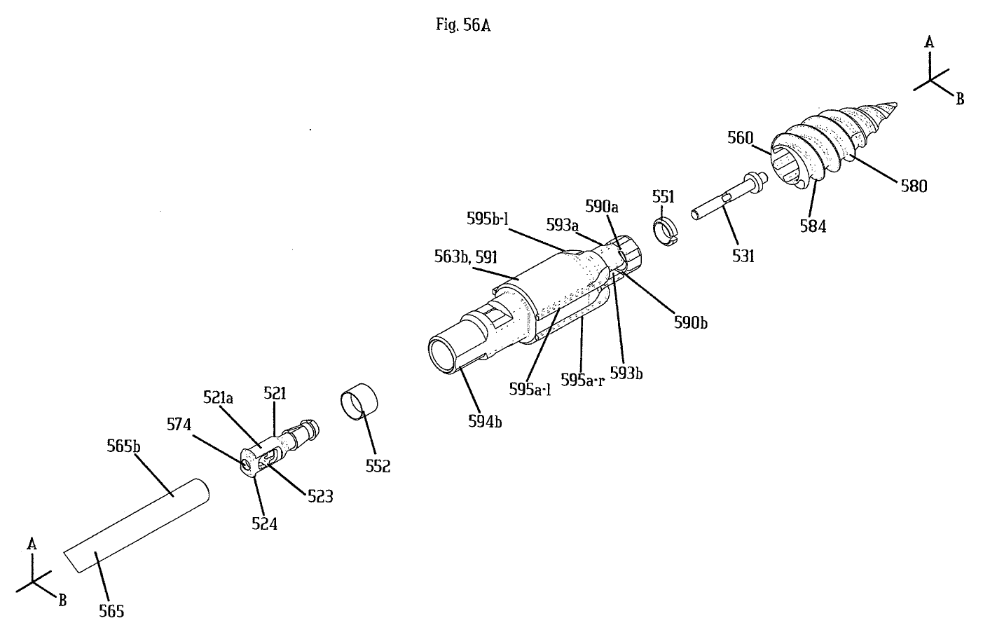

More particularly, referring now to FIGS. 54A, 54B, 55A, 55B, 56A, 56B, and 56C, the implantation tool 561 is shown in perspective view in FIG. 54A and in cross sectional side view in FIG. 54B. FIGS. 55A and 55B are a close up cross-sectional side view and an exploded view, respectively, of the proximal portion of the tool, FIGS. 56A, 56B, and 56C are an exploded perspective view and two orthogonal cross-sectional side views, respectively, of the distal portion of the tool with a bone anchor mounted thereon. The tool 561 can be considered to comprise four main parts. They are a shaft 563 with a through bore, a handle 564 attached to the proximal end of the shaft 563, a rod 565 extending through the bore of the shaft 563, and a nut 566 positioned inside a longitudinal bore 567 in the handle 564 and threaded onto the proximal end 563a of the shaft 563. The handle 564 is fixedly attached to the shaft 563. In the illustrated embodiment, for instance, two pins 575a, 575b pass through lateral holes 578a, 578b in the handle and occupy mating depressions 577a, 577b in the shaft 563 so that the shaft 563 cannot rotate or move longitudinally relative to the handle 564. The two pins 575a, 575b form an interference fit with their respective holes 578a, 578b and, therefore, are essentially fixed to the handle 564.

The handle may include two cleats 576a, 576b that can be used for temporarily securing the tensioned ends of sutures after passing through the eyelet of the eyelet pin, thus freeing one of the hands of the surgeon during implantation, as will be described in further detail below.

Referring to the cross-sectional and exploded views of FIGS. 55A and 55B of the proximal end 561a of the implantation tool 561, the proximal end 563a of the shaft 563 bears external threads 568 designed to accept mating internal threads 569 in a longitudinal distal blind bore 570 in the nut 566. The proximal bore 571 is configured in cross-section to accept the head of a torquing tool, e.g., a screwdriver or nut driver (not shown) that will be used to rotate the nut 566 relative to the shaft 563 within the handle. The cross-section of the proximal longitudinal bore of the nut may, for instance, be hexagonal so as to accept the head of a hexagonal driver. The rod 565 inside the shaft 563 extends past the proximal end 563a of the hollow shaft and into the distal bore 570 of the nut 566. An optional bushing 573 may be placed on the proximal end of the rod that provides an interface between the nut and the rod so that the rod 565 will not rotate when the nut 566 is rotated.

With this configuration, when the nut 566 is rotated, the mating threads 568, 569 of the distal bore 570 of the nut 566 and the proximal end 563a of the shaft 563 will cause the nut 566 to travel longitudinally relative to the shaft 563. Assuming the use of standard right handed threads, clockwise rotation of the nut 566 (as viewed from above the nut) will cause the nut to walk down the shaft 563, thus pushing the rod 565 distally out of the distal end 563b of the hollow shaft 565. Counterclockwise rotation of the nut will cause the nut 566 to walk proximally up the shaft 563. However, counterclockwise rotation of the nut 566 will not necessarily draw the rod 565 proximally because the rod is not mechanically attached to the nut, it only abuts it.

Referring to the cross-sectional side and exploded views of FIGS. 56A, 56B, and 56C of the distal portion of the implantation tool 561, the tool 561 preferably is delivered to the surgeon with the anchor 581 fixedly mounted on it. As will become clear from the following discussion, the distal end 563b of the shaft 563 is fixedly coupled to the anchor main body 580 of the anchor 581 via the retaining ring 541. Specifically, the retaining ring 541 is fixed both to the anchor main body 580 (by virtue of being in an interference fit within the longitudinal bore 560 of the anchor main body 580) and also to the tool 561 (by virtue of the retaining ring 541 being integrally formed as a frangible part of the distal end 563b of the shaft 563 of the tool 561 in the pre-surgical condition).

The entire shaft 563 of the tool may be a single, monolithic piece. However, in this particular embodiment, as can be seen in FIGS. 56A, 56B, and 56C, the distal-most portion of the shaft 563 comprises a separate, titanium piece 591 that may be fixed to the main portion of the shaft 563 by any reasonable means. The distal portion 563b of the shaft 563 is a separate piece 591 in this particular embodiment because a portion of it (namely, the retaining ring 541) is an implantable piece and, therefore, needs to meet the requirements for human implantation. The rest of the shaft, however, is not implantable, and therefore can be made of a different material. Of course, in other embodiments, the entire shaft 563 can be made of implantable-grade material and thus be monolithic.

Also, in the pre-surgical condition, the distal end 565b of the rod 565 abuts the proximal end 521a of the eyelet pin 521 in the anchor 580. Although not included in the illustrated embodiment, a nub may be provided at the distal end 565b of the rod for engaging the bore 574 in the proximal end 521a of the eyelet pin 521 for alignment purposes. Thus, when the rod 565 is pushed distally relative to the shaft 563 by clockwise rotation of the nut 566, the rod 565 pushes the eyelet pin 521 distally relative to the shaft 563, the anchor main body 581, and the central pin 531, all of which are essentially longitudinally fixed relative to each other by means of the retaining ring 541. On the other hand, if the rod 565 moves proximally relative to the shaft 563, the central pin 531 is unaffected (because the abutting nature of the interface between the distal end 565b of the rod 565 and the proximal end 521a of the eyelet pin 521 allows only for pushing of the eyelet pin 521 in the distal direction, and not pulling in the proximal direction by the rod 565).

The outer radial surface 563c at the distal end 563b of the shaft 563 is formed with a pattern to mate with a pattern 584 in the largest and most proximal portion 560c of the internal bore 560 of the anchor main body so that the twisting of the handle 564 and shaft 563 also twists the anchor main body 580 when the tool 561 is mounted to the anchor 581. Thus, the bore anchor 581 may be screwed into bone by twisting the tool 501 (at the handle 564).

The retaining ring 541 is frangibly attached to the distal end 563b of the shaft 563 by one or more breakaway portions. In this particular embodiment, the breakaway portions comprise two breakaway portions 590a, 590b positioned 180° radially from each other around the circumference of the shaft 563. The thickness, shape, and length of the breakaway portions 590a, 590b are designed to cause the retaining ring 541 to break off from the shaft 563 when a longitudinal force greater than a predetermined force, that is less than the interference force between the retaining ring 541 and the anchor body 580, is applied to the shaft 563, as will be described in greater detail below. Optionally, another, friction ring 552 may be positioned over the eyelet pin 521 over the frangible portions 590a, 590b just proximal of the retaining ring 541 to absorb any force loading that might otherwise load the frangible portions 590a, 590b prematurely. For instance, during assembly, lateral loading may occur that might break the frangible portions 590a, 590b. With the friction ring 552 in place, the friction ring 552 will take those loads rather than the frangible portions. If used, the inner radial surface of friction ring 552 is frictionally engaged with the outer radial surface of the eyelet pin 521 to hold it in place. As best seen in FIG. 56C, the proximal end of the retaining ring 541 may include an inner radial groove 553 for accepting the distal end of the friction ring 552 in the groove and trapped between the outer radial surface of the eyelet pin 521 and the side wall of the groove 553. The friction ring 552 may be made of a compressible material and have a wall thickness (at least at its distal end) slightly larger than the width of the groove between the side wall of the groove and the outer radial surface of the eyelet pin so as to form a compression fit in the groove 553.

The eyelet pin may be formed to have an overhang (or widened portion) 524 at its proximal end to help retain the retaining ring 541 and/or friction ring 552 within the anchor body. Further, as will be discussed further below, the bone anchors of the present invention are redeployable and intra-operatively adjustable and the widened portion 524 can also serve as a guide for an adjustment and/or redeployment tool that must be inserted into the implanted anchor to adjust or remove it.

The distal portion 563b of the shaft 563 has a discontinuous radial surface. Particularly, it includes slots 593a, 593b disposed 180° radially from each other around the circumference of the shaft 563. The slots 593a, 593b should be aligned radially with the eyelet openings in the eyelet pin 521 when the anchor 581 is mounted on the tool 561. Accordingly, any sutures and a suture shuttle may pass through the eyelet 523 in the eyelet pin 521 without interference from the tool 561 during any of the implantation and tissue attachment processes as described hereinabove or hereinbelow. Furthermore, the outer surface of the shaft 563 includes two flats 594a, 594b (they could also be grooves) running longitudinally along the length of the shaft and positioned 180° radially from each other around the perimeter of the shaft and aligned with the aforementioned slots 593a, 593b. Each of these grooves 594a, 594b provides a defined channel within which a suture or suture shuttle may run up to the handle 564 and be tensioned onto the cleats 576a, 576b of the handle. Accordingly, the cleats 576a, 576b, the longitudinal grooves 594a, 594b, the slots 593a, 593b, and the opposing ends of the eyelet 523 of the eyelet pin 521 are all radially aligned with each other.

FIGS. 56A, 56B, and 56C also show an exemplary optional suture shuttle 601 not shown in FIGS. 54A-55B. The suture shuttle is in the form of a ribbon (to be described in greater detail hereinbelow) passing through the slots 593a, 593b, and eyelet in the eyelet pin 521, and up along the grooves 594a, 594b on the shaft 563. As best seen in FIGS. 56A and 56C, the proximal edges 593a-1, 593b-1 of the slots 593a, 593b are beveled or angled to allow the suture shuttle 601 to bend gradually and less tortuously as it passes through the tool and eyelet. In accordance with another option shown in FIGS. 56A and 56C, a pair of rails 595a-1, 595a-r may be provided on opposing sides of flat portion 594a and another pair of rails 594b-1, 594b-r may be provided on opposite sides of flat portion 594b at tool 561 to further protect the suture shuttle 601 and any sutures from binding as they are shuttled through the tool and eyelet. Particularly, if the bone anchor is implanted below the surface of the bone, the slots 593a, 593b may be beneath or very close to the bone such that the suture shuttle 601 or sutures could be squeezed between the bone and the side of the tool and, therefore, difficult to pull through the tool and eyelet. The rails on each side of the suture shuttle 601 will assure a sufficient channel for the suture shuttle and sutures to slide freely.

An exemplary use of the anchor and implantation tool in accordance with this embodiment will now be described. As previously noted, the apparatus is preferably delivered to the surgeon in the pre-surgical condition with the anchor 581 affixed to the implantation tool 561. Particularly, as previously noted, the retaining ring 541 is frangibly attached to the distal end of the hollow shaft 563 of the implantation tool 561 (e.g., via the breakaway portions 590a, 590b) and also is affixed to the anchor main body 580 by virtue of being in an interference fit within the anchor main body's longitudinal bore portion 560a.

Once the surgical site is prepared and the bone exposed, the surgeon screws the anchor 581 into the bone with the tool by grasping the handle 564 of the implantation tool 561 and twisting it to screw the bone anchor main body 580 into the bone. The screwing of the anchor 580 into the bone does not load the breakaway portions 590a, 590b because all of the load is borne by the mating internal formation 584 on the anchor main body 580 and external formation 563c on the outer surface of the distal end 503b of the shaft 563. Next, with the tool 561 still attached to the now implanted anchor 581, sutures are attached to tissue and passed through the eyelet 523 of eyelet pin 521 of the anchor such as described in connection with the various surgical procedures discussed above in this specification.

When the surgeon has pulled the sutures through the eyelet 523 and tensioned them to the desired tension with the tissue in the desired position, the surgeon can temporarily secure the tensioned sutures to the cleats 576a, 576b in the handle 564 (FIG. 54A), thus freeing a hand. Now, the eyelet pin 521 is ready to be driven distally into the anchor main body 580 and over the central pin 531 into the closed position, thereby locking the sutures in the eyelet 523. This is accomplished as follows. First, the surgeon slips a torquing tool into the proximal bore 571 of the nut 566. While holding the handle 564 of the implantation tool 561 steady, the surgeon turns the torquing tool clockwise to cause the nut 566 to walk down the threads 568 at the proximal end 563a of the shaft 563. This forces the rod 565 to move distally relative to the shaft 563 since its proximal end 565a is abutting the distal end of the distal bore 570 of the nut 566. Since the distal end 565b of the rod is abutting the proximal end 521a of the eyelet pin 521 and the shaft 563 is fixed to the anchor 581, as long as the breakaway portions 590a, 590b are still intact, this drives the eyelet pin 521 down over the central pin 531 into the anchor main body 580 and into the closed position. As previously described in connection with the embodiments of FIGS. 36-41, the eyelet pin 521 is substantially fixed to the central pin 531 by virtue of an interference fit between the distal bore 522 of the eyelet pin 521 and the outer peripheral surface of the central pin 531. This fixation is overcome by the significant longitudinal force applied to the eyelet pin 521 via the torquing tool through the substantial leverage provided by the mating threads 568, 569, which convert a relatively small torque force into a substantial longitudinal force. As the eyelet pin 521 is pushed down over the central pin 531, the second ramp formation 524 of the eyelet pin 521 eventually passes through the C-ring 551, which causes the C-ring to expand to allow the ramp formation 524 to pass through and then snap shut once the second ramp formation 524 completely clears the C-ring 551. At this point, the eyelet pin 521 is now trapped in the closed position with the one or more sutures trapped in the eyelet 523 because the suture(s) are captured between the central pin 531 and an internal surface of the eyelet 523 as previously described in connection with the embodiments of FIGS. 36-41.

In any event, the eyelet pin 521 will eventually bottom out in the anchor, i.e., the distal end of the eyelet pin 521 will eventually hit the upper surface 533a of the shelf 533 of the central pin 531 and, therefore, be unable to move distally any farther relative to the central pin 531. At that point, continued clockwise turning of the nut 566 will attempt to move the nut 566, rod 565, and eyelet pin 521 relative to the shaft 563. However, since the eyelet pin 521 can no longer travel distally relative to the central pin 531 and anchor main body 580 once it has bottomed out on top surface 533a of the shelf 533, the continued clockwise twisting of the nut 566 will instead attempt to cause the shaft 563 to start moving proximally relative to rod 565 and eyelet pin 521. As the system now has no mobility, any clockwise rotation of nut 566 will load the system longitudinally due to the significant mechanical advantage of the screw threads acting upon the rod 565. The weakest structural portions in the assembly are the breakaway portions 590a and 590b, which are being loaded in longitudinal tension. This will cause the breakaway portions 590a, 590b to fail, thereby detaching the retaining ring 541 from the shaft 563.

The breakaway portions 590a, 590b are designed to break in response to a longitudinal force that is less that the longitudinal force needed to pull the retaining ring 541 out of its interference fit within the longitudinal bore 560 of the anchor main body 580. In one embodiment, the breakaway portions 576a, 576b are designed to fail at a force of about 150 pounds through manual, relatively low torsional force being applied to the nut 566.

At this point, the tool 561 is now detached from the anchor 580 and can be removed. The surgery can now be completed in the usual fashion.

The sutures may now be released from the cleats and the excess suture may be cut.

In accordance with the above description, it should be clear that yet another advantage of this particular embodiment is that the eyelet pin 521 is driven down over the central pin 531 slowly and atraumatically, rather than being hit with a mallet or other traumatic striking tool as was described earlier.

Ninth Set of Exemplary Embodiments

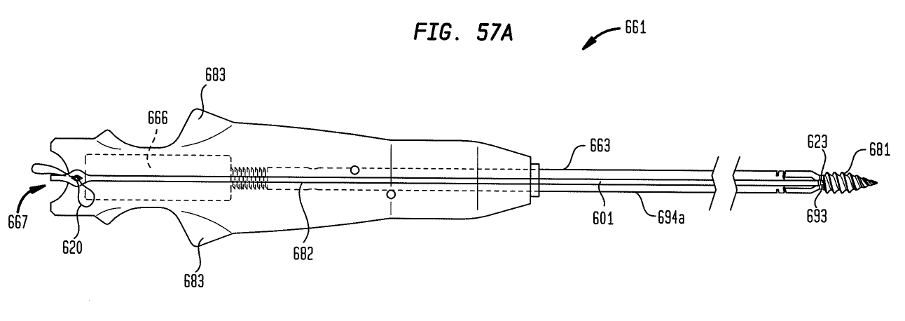

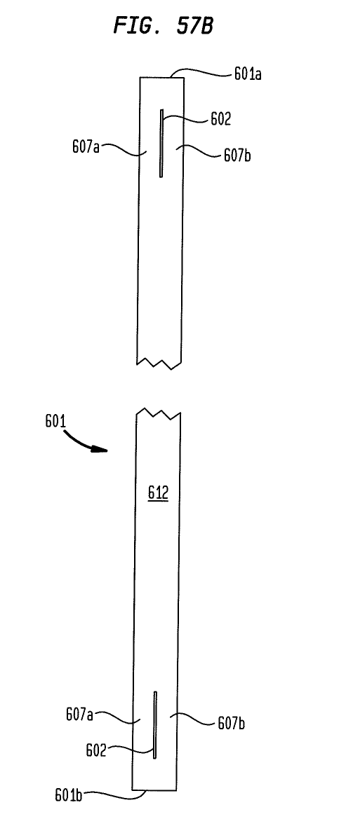

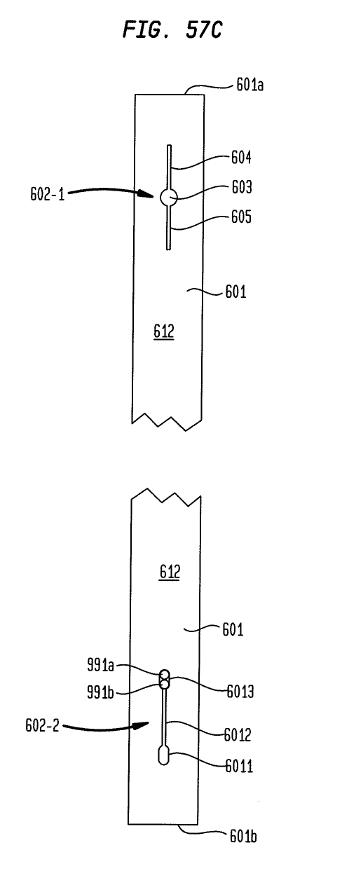

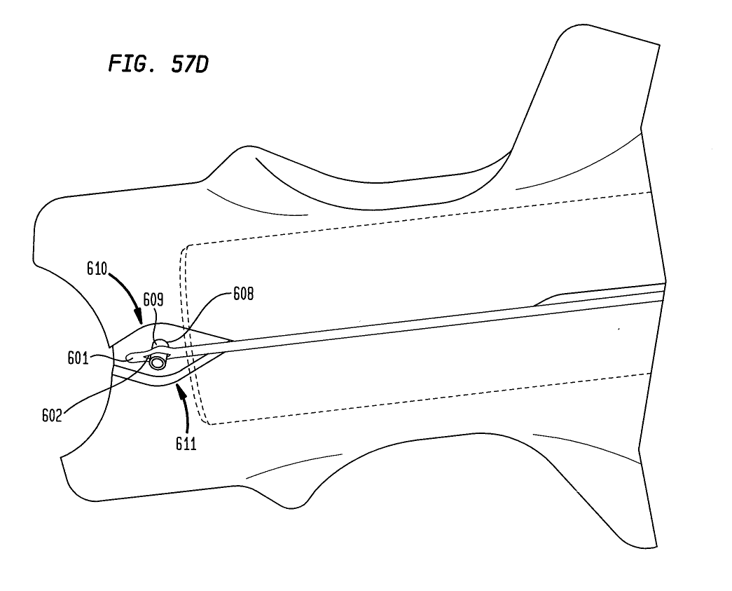

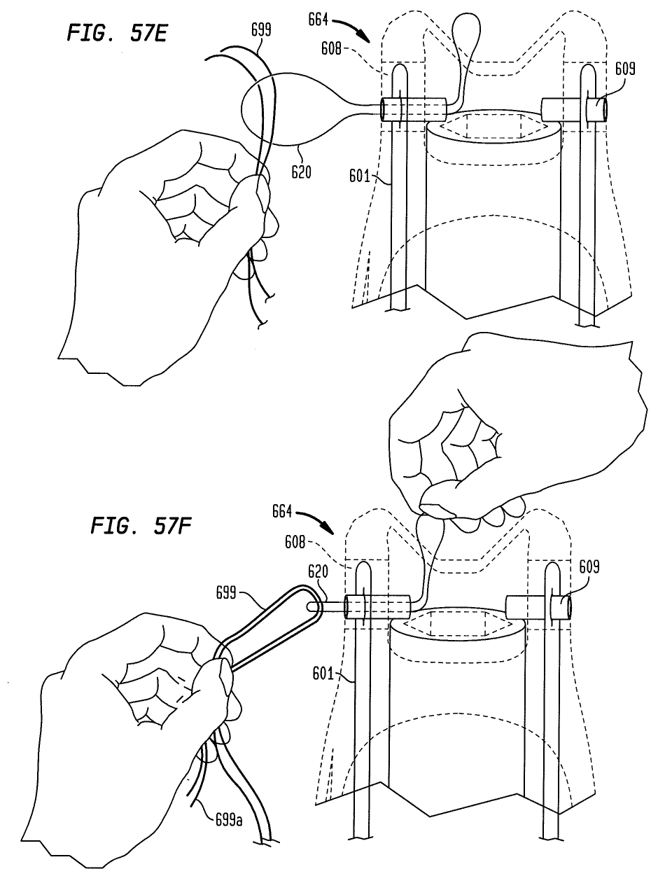

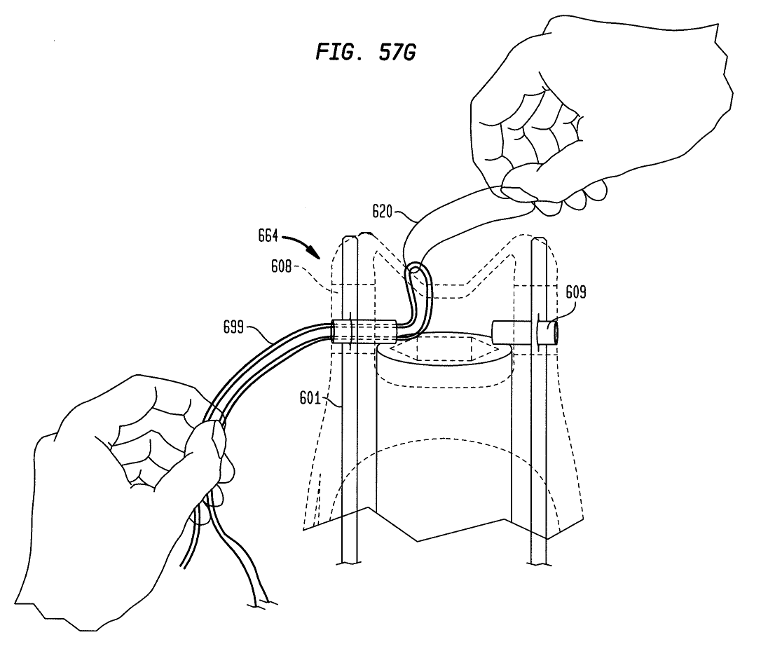

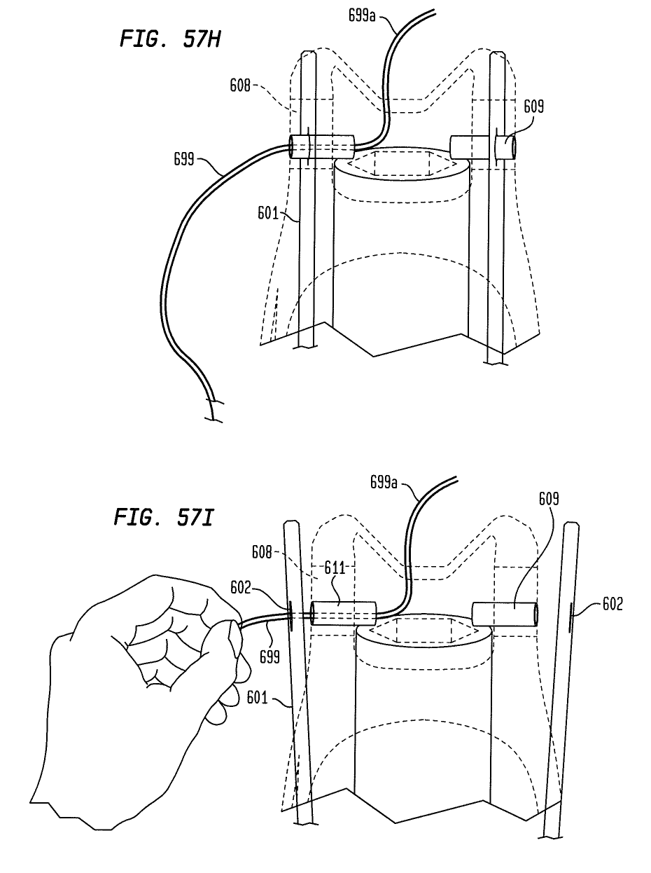

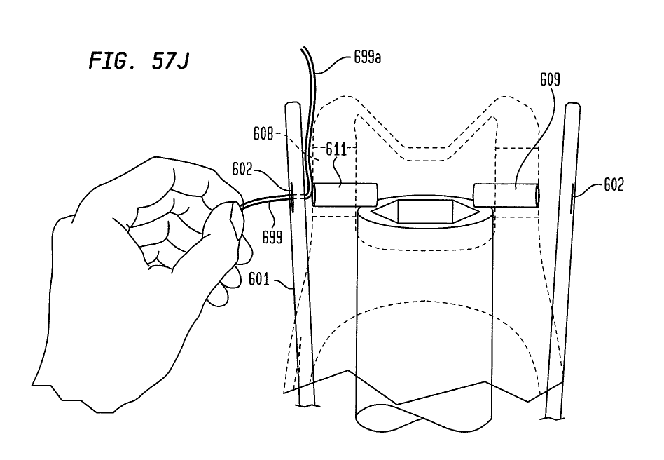

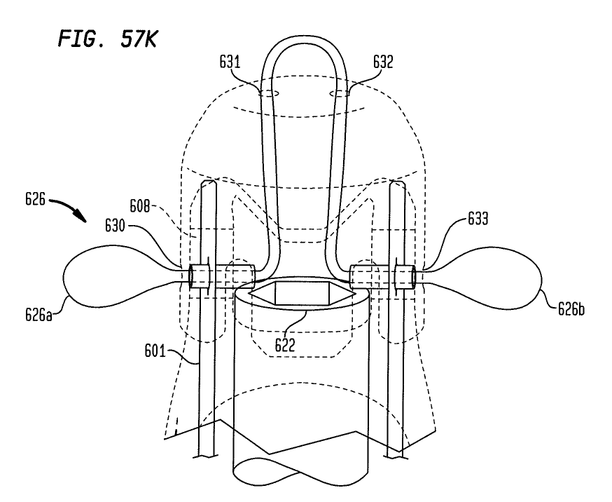

FIGS. 57A-57K illustrate another alternate set of embodiments in accordance with the present invention and including an embodiment of a suture shuttle such as the one briefly mentioned above in connection with FIGS. 56A-56C.

With reference first to FIG. 57A, an implantation tool 661 is shown bearing a bone anchor 681. The bone anchor 681 may be substantially the same as bone anchor 581 of the embodiments discussed in connection with FIGS. 52A-56C. The tool 661 can be considered to comprise four main parts. They are: a shaft 663 with a through bore, a handle 664 fixedly attached to the proximal end of the shaft 663, a rod 665 extending through and slidable within the bore of the shaft 663, and a nut 666 positioned inside a longitudinal bore 667 in the handle 664 and threaded onto the proximal end 663a of the shaft 663. The distal end of the tool 661 may be substantially similar to the distal end of the tool 561 disclosed in connection with the embodiments of FIGS. 52A-56C. The primary differences between the bone anchor and implantation tool in this embodiment relative to the embodiments of FIGS. 52A-56C pertain to the suture shuttle 601.

In this embodiment, the shaft 663 of the tool 661 has two flat portions 694 running longitudinally along the length of the shaft and positioned 180° radially from each other around the perimeter of the shaft (only one flat portion is actually visible in FIG. 57A) aligned with slots 693 in the distal portion of the shaft 663 that, in turn, align with the eyelet 625 in the eyelet pin when the anchor 681 is mounted on the tool 661 similarly to the embodiments of FIGS. 52A-56C. This configuration allows any suture or suture shuttles to pass through the eyelet in the eyelet pin without interference from the tool 661.

In the illustrated embodiment, the handle 664 does not include cleats, as was the case in the embodiment of FIGS. 52A-56C. However, the handle may include such cleats. Some surgeons may prefer cleats for temporarily securing sutures and others may not, preferring to wrap the sutures around their index fingers and pull up to tension the sutures while actuating the device to lock the sutures in the eyelet pin. In the illustrated embodiment, rather than cleats, the handle includes two large thumb rests 683. These thumb rests provide a substantial surface on which the surgeon may place his or her thumbs to provide a purchase against which to apply the pressure to pull up on the sutures with his or her index fingers.

The handle 664 includes two grooves 682 aligned with the flats 694 in the shaft, which grooves may be used for retaining a suture shuttle as will be described further herein below. Two apertures 611 are positioned on each side of the handle near the handle's proximal end aligned with grooves 682, respectively. As will become clear from the ensuing discussion, the apertures define an inner opening through the wall of the handle through which a suture may be passed and an outer surface onto which a slit in a suture shuttle may be mounted. Thus, for instance, the aperture may be as simple as a tube extending through a hole in the wall of the handle with the bore of the tube comprising the inner opening and the outer wall of the tube comprising the outer mounting surface for the suture shuttle.

FIG. 57B shows an exemplary suture shuttle 601 in accordance with one embodiment. The suture shuttle may comprise a ribbon of flexible material, such as metal, particularly an alloy of nickel and titanium, Nitinol™, spring tempered steel, polymer, a woven fabric or plastic material, or any flexible member capable of performing as described. The ribbon has a first end 601a and a second end 601b. A slit, hole, or other form of opening 602 (hereinafter “slit” or “opening”) is positioned in the suture shuttle 601 close to each end 601a, 601b. As will become clear from the ensuing discussion, the slits 602 at each end of the suture shuttle 601 will permit sutures to be attached to one of the slits 602 in the shuttle 601, which shuttle will be used to pass sutures through the eyelet in the eyelet pin in either direction. However, in other embodiments, a slit 602 may be provided near only one end of the shuttle. Each slit 602 is designed to have one or more of the sutures-to-be-shuttled pass there through for purposes of being shuttled through the eyelet. The entire opening may comprise a simple slit 606, smaller in width than the diameter of a suture, such as a laser cut slit of nominal width (e.g., 0.003 in.). Particularly, as will become clear in the following discussion of the use of the implantation tool of this embodiment, one or more sutures-to-be-shuttled may be inserted through the slit while the slit 602 is held open. The slit is held open by displacing the portions 607a, 607b of the suture shuttle ribbon that are on either side of the slit 602 in a direction perpendicular to the major surface 612 of the ribbon so that they are not coplanar with each other, thus opening the slit to allow sutures to pass through (see FIG. 57A).

In at least one alternate embodiment of the slit in the suture shuttle 601 as illustrated in the top half of FIG. 57C, the slits 602-1 may comprise three portions. The middle portion 603 may be an opening, such as a generally circular opening, large enough to freely accept at least one, and preferably, multiple sutures. At each end of the middle portion 603 is a narrowed portion 604, 605, the width of which is less than the thickness of each suture that is to be shuttled using the suture shuttle 601. As will be described in more detail below, sutures may be caused to enter the slit 602-1 relatively easily through the middle portion 603 and then tugged on to force them into the narrowed portion 604 or 605, whereupon they will become securely longitudinally captured in the opening 602 by the edges of the slits. The sutures may be released by tugging them back down into the middle portion 603 of the slit 602-1. As will become clear in the following discussion, narrowed portions 604, 605 also facilitate a certain type of bending or deformation of the slits for purposes of mounting the suture shuttle to the implantation tool 661 via the slits 602-1.

In accordance with another possible alternative embodiment as illustrated in the bottom half of FIG. 57C, the slits 602-2 may comprises three portions 6011, 6012, and 6013. Specifically, the middle portion 6012 may be a narrow slit portion, with slightly wider slit portions 6011 and 6013 towards either end of the slit 602-2. All three portions, however, are narrower than the sutures that will be placed through the slit 60-2-2. Preferably, the slit portions 6011 and 6013 have a length equal to or slightly less than the diameter of two sutures (e.g., sutures 991a and 991b) to better hold the sutures in the slits. Particularly, the edges of the slit bearing against the sutures helps keep the sutures from sliding out of the slit. Such a length maximizes the surface area of the sutures that it in contact with the edge of the slit. This embodiment also may decrease the possibility of the slit accidentally being ripped and also may better facilitate the bending or deformation of the slits for purposes of mounting the suture shuttle to the implantation tool 661 via the slits, as will be discussed in more detail below.

Different portions of the suture shuttle may be made of different materials to impart different stiffnesses as may be desirable for different applications. For instance, it may be desirable for the material properties of the suture shuttle to differ in the region of the slits as compared to the elsewhere because the inherent resilience of the slits is relied upon to secure sutures therein, whereas the rest of the suture shuttle does not need to serve such a function. Accordingly, the ends of the suture shuttle near the slits may be reinforced or made of different material than the remainder of the suture shuttle,

The apertures 611 positioned on each side of the handle 664 near the handle's proximal end aligned with grooves 682, respectively, are shown in the illustrated embodiment as comprising small holes 608 in the handle near the proximal end of the handle with short tubes 609 extending there through. A diamond shaped indent 610 is formed in the handle surrounding each aperture. In other embodiments, the aperture may be entirely integral with the handle. The aperture may be round, oval, diamond shaped or otherwise. However, an aperture having an oblong shape, such as a diamond or an oval, closely emulate the shape that the openings 602 on the suture shuttle 601 will take when mounted on the aperture, as will become clear from the discussion below. Accordingly, such oblong shapes may place less stress on the material of the suture shuttle when mounted on the tool.

Referring again to FIG. 57A, in the pre-surgical state, the entire implantation tool 661, anchor 681, and suture shuttle 601 are delivered to the surgeon preassembled. Particularly, the anchor 681 is attached to the implantation tool 661 essentially as described above in connection with the embodiments of FIGS. 52A-56C. The suture shuttle 601 is of a length such that it may have one of its slits 601 mounted over an aperture 611 on the handle and extend from that aperture 611, down through one groove 682 on a first side of the handle, pass over the flat portion 694 on that side of the shaft 663, into the slot 693 on that side of the distal end of the shaft 663, through the eyelet 625 in the eyelet pin 621 of the anchor 681 and back up through the other slot 693, over the flat portion of 694 on the second side of the shaft 663, through the groove 682 in the handle on the second side of the tool 600, and up to the other aperture 611 of the tool with the other slit 602 of the suture shuttle mounted over the other aperture 611.

The ends of the suture shuttle 601 adjacent the slits 602 are deformed to bend the portions 607a, 607b of the ribbon on opposite sides of the openings 602 away from each other in a direction perpendicular to the major surface 612 of the ribbon 601 so that portions 607a, 607b are not coplanar and the openings 602 are mounted on the apertures 611 in the handle. The last few millimeters of the suture shuttle 601 adjacent the ends 601a, 601b will likely twist about 90° to accommodate this deformation and mounting on the aperture 611.

In the alternate embodiment of the slit 602 illustrated in FIG. 57C (comprising round opening portion 603 and narrow portions 604 and 605, if the round opening portion is designed to be only slightly smaller than the outer diameter of the tubes 609, then the ribbon may not twist to accommodate mounting on the apertures 611. Rather, the edges of round opening portion 603 may simply flare outwardly from the plane of the major surface 612 of the ribbon. The narrowed portion 604, 605 of the slit 602 help permit the flaring without damaging or permanently deforming the ribbon.

Since the material of the ribbon is resilient, the slits 602 want to close (i.e., return to their unstressed shape) in which the portions 607a, 607b of the ribbon on either side of the slit 602 return to the coplanar position and minimize the slit opening size. Due to this tendency, segments 607a, 607b of the suture shuttle essentially squeeze the apertures 611, thereby relatively tightly holding the suture shuttle 601 on the apertures 611.

Mounting the slits 602 over another structure, such as the tubes 609, also prevents the edges of the slits 602 from contacting the sutures-to-be-shuttled as they are being pulled through the opening. Particularly, the edges of the slits may be sharp and could damage a suture as it is pulled through.

In order to facilitate the loading of sutures-to-be-shuttled through the slits 602 in the suture shuttle 601 so that such sutures may be shuttled through the eyelet of the bone anchor using the suture shuttle, one or more wire loops 620 may be disposed through the apertures 611 in the handle (and thus through the openings 602 in a suture shuttle 601 that is mounted on the apertures 611 as described above). The wire loops 620 may be closed loops (e.g., a circle of wire) as shown in the Figures or open loops (e.g., a length of suture folded over on itself). The term “wire” in the context of the wire loops use for loading sutures into a suture shuttle is being used generically. The loops 620 may be formed of any flexible material, including metal wire, suture, nylon string, etc.

As will be described in more detail below, any sutures 699 that are to be shuttled by the suture shuttle 601 through the eyelet 625 in the eyelet pin 621 of the anchor 681, first must be loaded through a slit 602 in the suture shuttle. Such sutures can be passed through the portion 620a of a wire loop 620 extending from the outer side of the aperture 611, as shown in FIG. 57E. Then, while holding on to the free end 699a of the suture-to-be-shuttled (so that the sutures do not slide out of the wire loop 620), the portion of the wire loop 620 extending from the inner side of the aperture 611 may be pulled on (see FIG. 57F) until the wire loop 620 is pulled completely through and out of the aperture 611 on the inner side, bringing the sutures-to-be-shuttled through the aperture 611 along with it (see FIG. 57G). At this point, a looped portion of each suture-to-be-shuttled 699 passes through the aperture 611 and slit 602 in the suture shuttle 601 with the free ends 699a of the sutures still on the outer side of the aperture 611 and slit 602. The surgeon may now release the free ends 699a of the sutures-to-be-shuttled 699 and pull on the loops 699b of suture to bring the free ends 699a of the sutures-to-be-shuttled through the opening 602 as shown in FIG. 57H. Having served its purpose, the wire loop 620 may be freed from the sutures-to-be-shuttled, 699, and discarded.

At this point, with reference to FIG. 57I, the sutures are fully threaded through the slit 602 in the suture shuttle (and the corresponding aperture 611). Next, the surgeon can remove the end 601a or 601b of the suture shuttle 601 from the aperture 611 with his or her finger. The opening 602 in the suture shuttle 601 will return to its original, undeformed shape and the sutures-to-be-shuttled will be captured in the slit 602 in the suture shuttle. (In the slit embodiment of FIG. 57B, the sutures-to-be-shuttled might not be automatically captured in the slit and might need to be pulled longitudinally into one of narrow portions 604, 605 to become longitudinally captured.) The free ends of the sutures-to-be-shuttled, having been attached to suture shuttle 601, can now be pulled completely back through the aperture 611 to completely free them from the handle 664, as shown in FIG. 57J.

FIG. 57K is a cross-sectional side view of an alternative embodiment of the proximal end of the tool 661 having a cap 622 that can be used to even further facilitate the loading of sutures-to-be-shuttled into the slits 602 of the suture shuttle 601. This cap 621 may be silicone, rubber, or another material that can be fitted over the proximal end of the handle 664 of the implantation tool 661. The cap 622 should be sized so as to require a minimal amount of stretching to fit over the end of the handle so that it will stay on the end of the handle by the force of friction between the outer surface of the handle and the inner surface of the cap, but be relatively easily removable by hand by a surgeon or nurse. The cap 621 has one or more wire loops 626 disposed in it that will be used similarly to the wire loops 620 in the FIG. 57A embodiment to facilitate the insertion of sutures into the openings 602 in the suture shuttle. In the particular embodiment illustrated in FIG. 57D, the wire loop comprises one long closed loop of suture 626.

In the illustrated embodiment, the single closed loop of suture 626 can be used to shuttle sutures through either of the two slits 602 in the suture shuttle. Particularly, loop 626 passes through one of the apertures 611 in the handle (and the associated slit 602 in the suture shuttle that is mounted on that aperture as well as through a slot 630 in the side of the cap 621 to accommodate the aperture 611). From there, the wire loop extends into the inside of the cap 621 up through a first hole 631 in the top of the cap, then back down through a second hole 632 in the cap, and through the other aperture 611 in the handle (including the other slit 602 in the suture shuttle that is mounted on that aperture and another slot 633 in the side of the cap 621 that accommodates that aperture 611). Accordingly, in appearance, the cap has two loop segments 626a, 626b extending from the cap as shown in FIG. 57K. The pictured embodiment is merely exemplary. There may be two separate wire loops instead of one. Also, there may be one hole in the top of the cap that the loop 626 passes out of and back into. In fact, the loop need not exit the cap at the top at all. This is merely one convenient way to provide some friction between the cap 621 and the loop 626 so that the loop is relatively fixedly attach the cap and will not accidentally be pulled out of the cap when loading sutures into the apertures as described in the next paragraph.

Now, if suture(s)-to-be-shuttled are passed through either loop segment 626a or 626b extending from the side of the cap, then, when the cap 622 is pulled off of the top of the tool handle, the suture(s)-to-be-shuttled that are passing through one of the segments 626a or 626b of loop 626, will be drawn through the aperture 611 in the handle (and thus through the corresponding slit 602 in the suture shuttle 601) essentially as described above in connection with the embodiment of FIGS. 57A-57K.

Alternately, the cap may be externally or internally threaded to the top of the handle. Unscrewing the cap also will cause the suture loop to be pulled through the apertures 611, bringing the suture(s)-to-be-shuttled through the aperture also, as previously described.

In operation, the suture shuttle 601 and any of the aforedescribed wire loop systems for loading sutures-to-be-shuttled into the slits 602 in the suture shuttle 601 facilitates ease of use of the implantation system. Particularly, in an exemplary arthroscopic procedure, the bone anchor 681 and implantation tool 661 may be inserted into the patient through a cannula and an incision in the patient's body. The anchor is fixed to bone as previously described in connection with any of the embodiments in this application. Then, through techniques well known in the art and/or disclosed in this application, sutures are brought up through the same cannula in which the implantation tool is inserted. The sutures may, for instance, be coupled to tissue (either directly or via one of the tissue fastener devices 2 disclosed in this specification), such as a rotator cuff that needs to be re-attached to the humerus bone via the bone anchor 681. In any event, the sutures are brought up through the cannula and inserted through one of the openings 602 in the suture shuttle 601, such as in any one of the manners described hereinabove using wire loop 620 or 626 and/or the cap 621. The sutures are longitudinally captured in place in the opening 602 (again such as in any of the ways previously described hereinabove).

Next, the end (e.g., 601a) of the suture shuttle 601 bearing the suture(s)-to-be-shuttled is removed from the aperture 611. The opposite end (e.g., 601b) of the suture shuttle also is removed from of its aperture 611. The suture shuttle 601 is now ready for deployment to draw the suture(s)-to-be-shuttled through the eyelet in the eyelet pin of the bone anchor 681. Particularly, the surgeon now pulls proximally on the end 601b of the suture shuttle opposite the end 601a in which the suture(s)-to-be-shuttled have been inserted. This, of course, draws the end 601a of the suture shuttle within which the sutures-to-be-shuttled are fixed down along the length of the tool handle 664 and tool shaft 663, through the eyelet in the eyelet pin, and back up along the diametrically opposite side of the tool shaft 663 and handle 664, carrying the suture(s)-to-be-shuttled with it. (Note that the suture shuttle also may be use to shuttle sutures from outside the body through the eyelet of the anchor in essentially the same manner for different procedures.) In fact, the suture shuttles described herein may be used for generally any type of suture shuttling or suture passing and is not limited to use with the tools described herein. Furthermore, it is not limited to uses involving the shuttling of sutures. It may be used to grasp and/or shuttle tendons, ligaments or any other generally longitudinal anatomical members. Because the suture shuttle can be made of a resilient material with some stiffness, such as Nitinol™, the suture shuttle, including the slits, may be fabricated to have an unbiased shape of any configuration that may be desirable for its particular purpose. Thus, in one alternate embodiment, the suture shuttle may be fabricated such that the slit or slits are normally open rather than closed when unbiased and can be biased closed as needed. For instance, such a suture shuttle may be provided within a tube, such as a catheter. When the shuttle needs to accept a suture through the slit, the end of the shuttle bearing the slit is extended from the end of the catheter so that the slit may rebound to its unbiased open position. After the suture is passed through the open slit, the shuttle may be retracted into the catheter, the lumen of the catheter shaped so that, when the slit is retracted within the catheter, the inner wall of the catheter lumen biases the slit closed, trapping the suture in the slit.

Furthermore, according to another alternate embodiment, it has been found that fabricating a slight curvature into the longitudinal ends of the suture shuttle (the radius of the curve being perpendicular to the major surface 612) while leaving the majority of the shuttle between the two ends straight facilitates the ease of pulling the suture shuttle through the eyelet. In one exemplary implementation, the entire suture shuttle is 572 mm long, the slits are 9 mm long and start 2 mm from the ends of the shuttle and the last 1.8 mm of each end of the shuttle is imparted with a curvature of radius 2 mm. Accordingly, in this embodiment, almost the entire length of the shuttle, including the slits, is flat and only the very ends (laterally outwardly of the slits) is curved.

In yet other embodiments, multiple suture shuttles may be mounted to the tool simultaneously to permit multiple sets of sutures to be shuttled through the eyelet at different times or locations. In fact, a plurality of eyelets may be provided in an eyelet pin and a plurality of suture shuttles may be mounted on the tool passing through the plurality of different eyelets.

Additionally, because the suture shuttle is in the form of a ribbon (i.e., has major surface 612 and a much thinner depth perpendicular to the major surface as well as has a stiffness, the suture shuttle as well as the sutures trapped in it will travel down the one side of the instrument, through the eyelet and back up the other side without any twisting about the longitudinal axis of the shuttle. Thus, the suture shuttle tracks smoothly and easily through the eyelet and the sutures do not twist around each other. A problem with some conventional suture shuttles made of braided filaments is that they tend to twist as they pass through a restricted passageway, such as the eyelet. The smaller the pitch of the braid, the more it tends to twist. This causes the sutures being shuttled to also twist around themselves, which can cause the sutures shuttled to bunch up where they are trapped in the slit of the suture shuttle so as to increase the cross section of the suture material that must pass through the eyelet, impeding smooth passage of the sutures and suture shuttle through the eyelet.

The aspect ratio of the width of the shuttle (e.g., left to right in FIGS. 57B and 57C) being much greater than its thickness or depth (e.g., into and out of the page in FIGS. 57A and 57B) is important to the performance of the shuttle. Particularly, the shuttle is resilient but relatively flexible parallel to its thickness, relatively stiff parallel to its width, and relatively resistant to twisting about its longitudinal axis (although, as mentioned above, it must be twisted about 90° to mount the slits over the apertures of the handle). The relative high flexibility parallel to its thickness is what allows it to bend and track easily down one side of the tool, through the eyelet, and up the other side. Its relative stiffness parallel to its major surface 612 keeps the shuttle in line with the tool. Finally, the resistance to twisting about its longitudinal axis keeps the shuttled sutures from twisting around themselves and/or the shuttle and bunching up as they are being shuttled.

In one embodiment, the suture shuttle is formed of NiTinol™ and is 0.25 mm thick and 1.5 mm wide, giving it an aspect ratio of about 6:1, which has been found to be quite suitable for this particular application. Preferably, the edges of the shuttle are rounded to prevent the person handling the suture shuttle from cutting his or her gloves or hands on any sharp edges.

With reference to FIG. 57J, it should be apparent that, in the illustrated configuration, when the suture shuttle is pulled to shuttle the sutures 699 through the eyelet, the distal ends 699a of the sutures will pass through the eyelet above the suture shuttle and the loop portion of the sutures will be below the ribbon. Some surgeons prefer to have the distal (free) ends of the sutures pass through the eyelet below the sutures shuttle and the attached ends above the suture shuttle. It is believed that this results in lower shuttling force, thereby facilitating the ease with which the sutures pass through the eyelet. The distal ends of the sutures can be made to pass through the eyelet above or below the sutures shuttle by virtue of selecting how the slit of the suture shuttle is mounted over the aperture 611. Particularly, as previously described, the suture shuttle must undergo a 90° twist near end of the shuttle adjacent the slit in order to allow the slit to be mounted over the aperture. Looking down from above on the end of the shuttle, this twist may be clockwise or counterclockwise. Likewise, after the twisting, when spreading open the slit to mount it over the aperture, there are two ways to spread open the slit. Particularly, looking down the barrel of the aperture from outside the tool, the portion of the shuttle on either side of the slit 607a or 607 (FIG. 57B) that is closer to the handle of the tool (i.e., the radially inward portion 6007a or 607b) may be spread to the right (while the radially outward portion 607b or 607a) is spread to the left or vice versa.

Hence there are four permutations of how the each slit may be mounted over the corresponding aperture, namely, (1) clockwise-twisted/inner portion 607 to the right, (2) clockwise-twisted/inner portion 607 to the left, (3) counterclockwise-twisted, inner portion 607 to the right, and (4) counterclockwise-twisted, inner portion 607 to the left. Of course, when the suture shuttle is released from the aperture, the shuttle and slit will return to their unbiased configurations with no twist and with the both portions 607a and 607b coplanar and with the distal ends of the sutures trapped in the slits either facing outwardly of the handle or inwardly of the handle. It should be apparent that, when the distal ends 699a of the sutures 699 face outwardly, they will pass through the eyelet below the shuttle and, when they face inwardly, they will pass through the eyelet above the shuttle. Two of these mounting options will result in the sutures passing through the eyelet with their distal ends 699a below the shuttle and two will result in the sutures passing through the eyelet with their distal ends 699a above the shuttle. More particularly, clockwise-twisted/inner-portion-right and counterclockwise-twisted/inner-portion-left will result in the distal ends 699a of the sutures facing outwardly from the handle and hence passing through the eyelet below the suture shuttle.

Those sutures are now through the eyelet in the bone anchor 681 and extending out of the patient's body. The surgeon can now release the sutures from the slit 602 in the suture shuttle 601. The manner in which the surgeon releases the sutures from the slit 602 in the suture shuttle may vary depending on the surgeon and/or the particular embodiment of the suture shuttle. For instance, in the exemplary embodiment of FIG. 57B, in which the suture shuttle opening comprises a middle section 603 that is larger than the diameter of the suture, the surgeon can merely push or pull on the sutures to force them out of the narrow slit 604 or 605 of the slit 602 and into the larger middle portion 603 of the slit and then pull them through until they are free of the slit 602. Alternately and particularly in the embodiment of FIG. 57A, in which the slit 602 is just a laser etched slit, the surgeon can simply cut the suture(s) and discard the suture shuttle 601 along with the end(s) of the suture(s) that are still trapped in the slit 602.

In any event, now the surgeon may pull the desired tension on the sutures to draw the soft tissue onto the bone surface adjacent the bone anchor 681, and then deploy the eyelet pin 621 to the closed position as previously described to lock the sutures in the bone anchor.

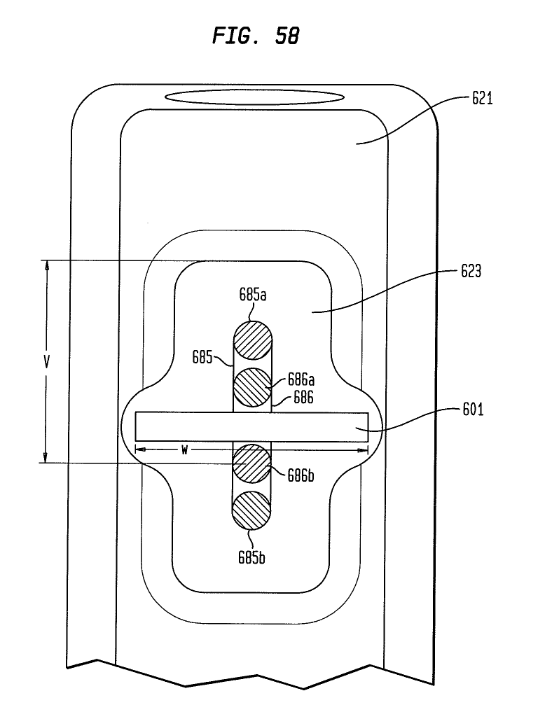

FIG. 58 is a close-up view of the slit 602 of the suture shuttle 601 passing through the eyelet 625 in the eyelet pin 621 during suture shuttling carrying two sutures 685 and 686. The eyelet 625 has a widened, ribbon-guiding portion 625a adapted to accept the suture shuttle ribbon 601 in a certain orientation. With reference to FIG. 58, an advantage of this embodiment is that the use of a flat ribbon as the suture shuttle 601 and an appropriately sized and shaped eyelet 625 relative to the width of the ribbon 601 guarantees that the ribbon 601 will pass through the eyelet 625 in a certain orientation and position (as shown in FIG. 58, e.g., with its width dimension, w, oriented horizontally relative to the bone anchor. The advantage of this is that the sutures 685, 686 that are fixed in the slit 602 are therefore guaranteed to pass through the eyelet 625 in the orientation shown in FIG. 58, namely, with the plurality of suture segments neatly stacked in the vertical dimension, V. It should be remembered that each suture captured in the slit 602 of the suture shuttle will be folded over on itself about the suture shuttle 601 as it passes through the eyelet. Thus, each individual suture actually passes through the eyelet 625 with one segment 685a, 686a of the suture above the ribbon 601 and one segment 685b, 686b below the ribbon 601. Thus, for instance, if two sutures are being shuttled, as shown, the eyelet 625 must accommodate four suture diameters in the vertical dimension, v. This configuration helps keep the sutures-to-be-shuttled 685, 686 from getting caught or binding as they pass through the eyelet 625.

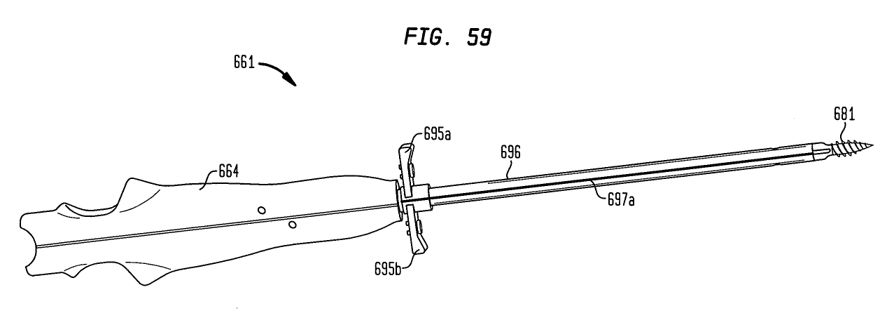

As illustrated in FIG. 59, the shaft 663 of the insertion tool 661 may be pre-surgically encased in a plastic sheath 696 in order to protect the tool shaft and other components and keep them organized during the surgical procedure. The sheath 696 may run from the proximal end of the anchor 681 all the way up to the distal end of the handle 664. The sheath can remain in place until the suture shuttle is ready to be deployed. The use of the sheath is advantageous as these procedures are usually performed arthroscopically through surgical ports. One can readily imagine that, if the sheath was not in place, while driving the anchor, the ribbon 601 may get caught up in either the surgical port or the tissue of the patient.

The sheath 693 may be formed to make it easily tearable for removal during the procedure. For instance, in one exemplary embodiment, the sheath 693 has two weakened strips 697a, 697b (of which only one is visible in the figure) running longitudinally along the sheath and diametrically opposed from each other. The weakened portion, for instance, may comprise portions of the sheath that are thinner than the remainder of the sheath. The sheath 696 also may include tabs 695a, 695b at the proximal end to permit easy grasping of the two sides of the sheath for tearing. By grasping the tabs 695a, 695b and pulling them away from each other in the direction transverse to the longitudinal axis of the tool 661, the sheath can be caused to tear away longitudinally at the two weakened sections 697a, 697b. If the surgeon either pulls upwardly as he is tearing or keeps his hand stationary in the longitudinal direction of the tool, then the sheath will simple slide upwardly along the shaft as it is torn.

It may be useful to permit slack in the suture shuttle for purposes of loading the suture shuttle 601 onto the apertures 611 in the handle 664. Particularly, if the suture shuttle is under tension, then it is more difficult to open the slits 602 for mounting on the apertures 611. However, after the suture shuttle is mounted on the apertures, slack is undesirable because it would cause the suture shuttle to bow outwardly from the tool shaft 663. Accordingly, providing the apertures on a spring-biased carriage in the handle would allow the carriage to be forced distally against the spring bias during assembly so that the openings on the suture shuttle can be mounted on the apertures 611 while there is slack in the suture shuttle 601. Then, when the force on the carriage is released, the spring will bias the carriage proximally along the handle 664, thus taking up any slack in the suture shuttle 601.

In accordance with one embodiment (not shown), one or more portions of the handle bearing the aperture 611 may be slidably mounted on a spring-loaded carriage relative to the rest of the handle. For instance, the carriage(s) may be mounted on rails in a slot in the handle and biased proximally by a spring. However, it can be forced distally within the slot.

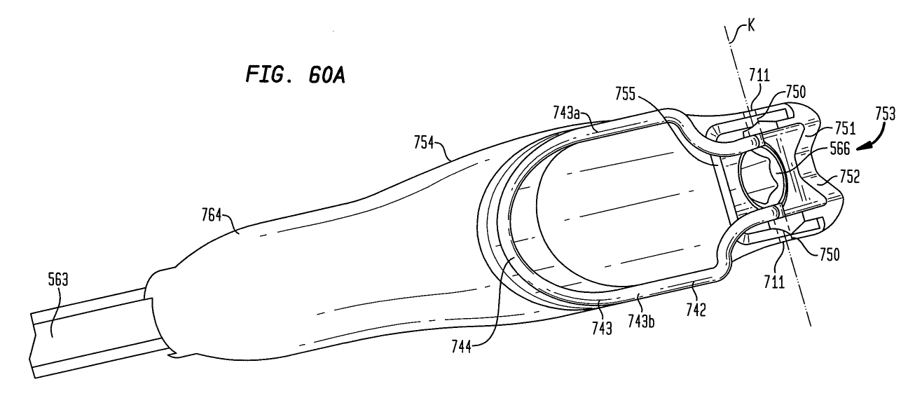

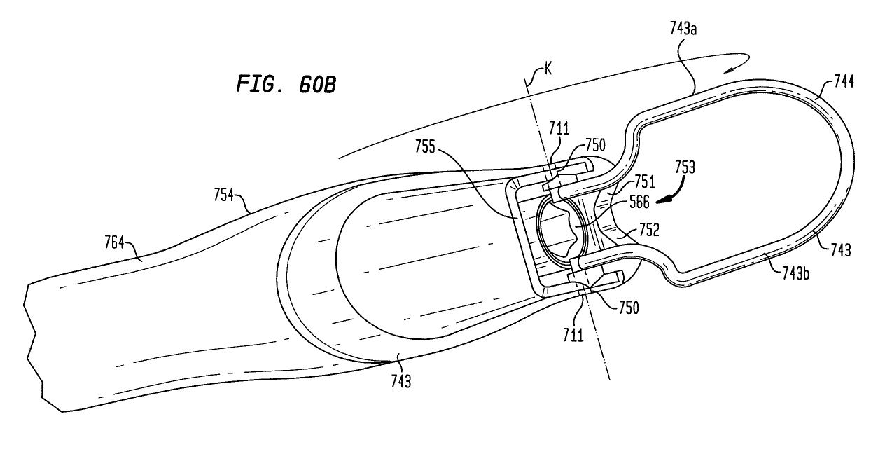

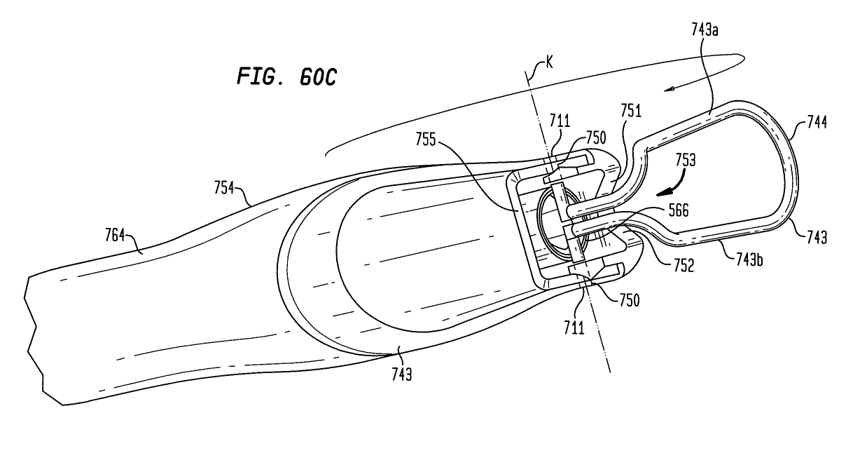

FIGS. 60A-60C illustrate an alternate embodiment of the proximal portion of an implantation tool 661b that provides both (1) an alternate suture loading mechanism that can provide slack in the suture shuttle 601 for purposes of loading the suture shuttle onto the apertures 711 and then taking up that slack after mounting and (2) a simple mechanism for releasing the suture shuttle from the apertures. This embodiment may include a cap such as cap 622, and/or one or more wire loop such as wire loops 620 or 626, for loading suture(s)-to-be-shuttled through the aperture 711 of this embodiment. However, these components are not shown in order not to obfuscate the features of interest on this embodiment. FIG. 60A is a perspective view of the handle 764 with the handle shown in see-through so as to permit viewing of the components inside the handle. The components inside the handle may be essentially identical to the components in the embodiments of FIGS. 57A-57K. In this embodiment, the apertures comprise part of a clip 742 mounted on the proximal end of the handle 764. Particularly, the clip includes two tubes 711 which serve as the apertures upon which the slits 602 of the suture shuttle are mounted. The clip may further include a lever portion 743 comprising two legs 743a, 743b and a gripping portion 744. In the particular embodiment illustrated in these figures, the apertures 711 are integral with the lever portion 743 and grasping portion 744 to form the overall clip 742. In fact, the entire assembly may comprise one wire form. The wire form is resilient in that the opposing ends of the wire form where the apertures 711 are may be squeezed inwardly towards each other in the direction of arrows j. The apertures 711 are mounted within vertically oriented slots 750 at the proximal end of the tool handle 764. Accordingly, the clip 742 can translate vertically relative to the tool handle with the apertures 711 sliding within the slots 750 in the handle.

The top of the handle (i.e., its proximal end) is shaped so as to provide cam surfaces 751, 752 for the legs 743a, 743b of the clip 742. The cam surfaces 751, 752 essentially comprise the edges of a generally U or V-shaped notch 753 in the sidewall 754 of the handle adjacent and open to the proximal end of the handle. In this particular embodiment, a generally rectangular opening 755 also is provided in the side surface of the handle opposite the U or V-shaped notch 753 open to the proximal end of the handle in order to provide clearance for the two legs 743a, 743b of the clip (without squeezing them together). The cam surfaces 751, 752 will force legs 743a, 743b inwardly toward each other if the clip is rotated about the axis k defined by the apertures 711 to cause the legs to ride on the cam surfaces 751, 752. Sutures-to-be-shuttled may be passed through the slit 602 in the suture shuttle and the aperture 711 on the handle using wire loops in the manner previously described in connection with the embodiments of FIGS. 56A-56K.

This design provides a mechanism for permitting slack in the suture shuttle 601 during loading of the suture shuttle on the apertures 711 and then taking up that slack. Particularly, as previously described in connection with the various embodiments of FIGS. 52A-58, nut 566 in FIGS. 54A, 54B, 55A, and 55B can be caused to travel longitudinally relative to the shaft 563 and handle 664 by the action of the mating screw threads on the shaft 563 and nut 566, respectively, as the nut is rotated. During loading of the suture shuttle 601 onto the apertures 711, the nut 566 may be positioned in the lowest (i.e., distal-most) position possible without driving the eyelet pin into the anchor main body and over the central pin. With reference to FIGS. 54A, 54B, 55A, and 55B, this, for instance, would be the position where the distal bore 570 of the nut is abutting the proximal end 565a of the rod 565 and the distal end 565b of the rod 565 is abutting the proximal end of the eyelet pin 521 but there is no force applied on the eyelet pin by the rod 565. The relative length of the tool and the suture shuttle 601 can be selected so that, when the apertures 711 are in this position, there is sufficient slack in the suture shuttle 601 to allow easy mounting of the suture shuttle 601 on the apertures 711.

Then, after the suture shuttle is mounted on the apertures, the nut 566 can be rotated counterclockwise (assuming right handed threads) to move the nut proximally. The proximal end of the nut 566 will push the apertures 711 proximally in their slots 750. The nut 566 can be moved longitudinally a distance to take up all of the unnecessary slack in the suture shuttle 601. Thereafter, the nut can be left in that position so that the apertures 711 cannot move back down distally. The tension in the suture shuttle itself will keep the apertures 711 from moving further proximally. This is the position shown in FIG. 60A.

Referring now to FIG. 60B, suture(s)-to-be-shuttled 776 may be loaded through the slot 602 in the suture shuttle 601 and the apertures in any of the manners described in connection with any of the previously described embodiments. When it is time to release the suture shuttle 601 from the handle 764 (i.e., after the shuttle has been loaded with one or more sutures-to-be-shuttled 776), the surgeon may grasp the gripping portion 744 of the clip 742 and rotate the clip about its pivot axis K defined by the longitudinal axes of the apertures 711. Eventually, the legs 743a, 743b will reach the cam surfaces 751, 752 and start riding against them as shown in FIG. 60B.

Referring now to FIG. 60C, as the legs 743a, 743b of the lever portion 743 ride against the cam surfaces 751, 752, they will be forced toward each other, causing the apertures 711 to approximate each other and eventually become free of the slot 750 (this is the position shown in FIG. 60C) so that the clip 742 is released from the handle 764 and can be removed. Thus, in this particular embodiment, rather than removing the slits 602 in the suture shuttle 601 from the apertures 711 in order to release the suture shuttle from the handle, instead, the apertures 711 are removed from the slits 602, thereby releasing the suture shuttle 601 from the handle 764.

Once the clip 742 is separated from the handle 764 (by removing the apertures 711 from the slots750), the free ends of the suture(s)-to-be-shuttled 776 are pulled back through the pass-through 711 and slot 750, thus releasing them from the pass-through.

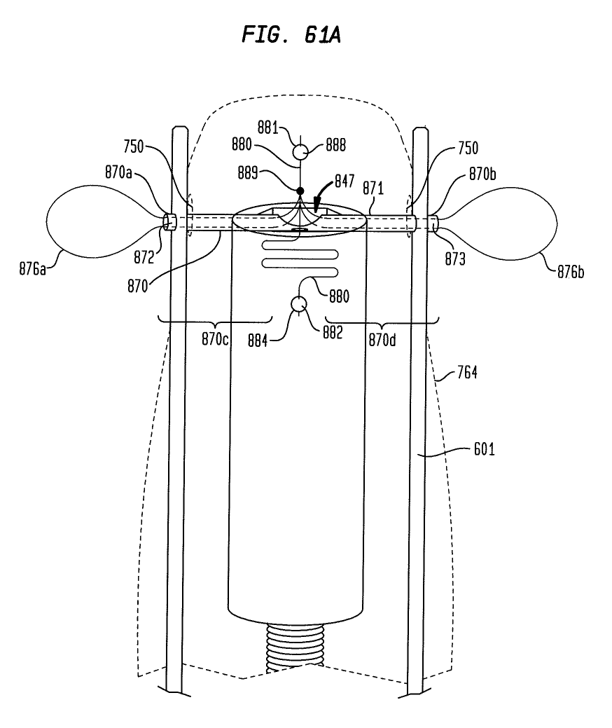

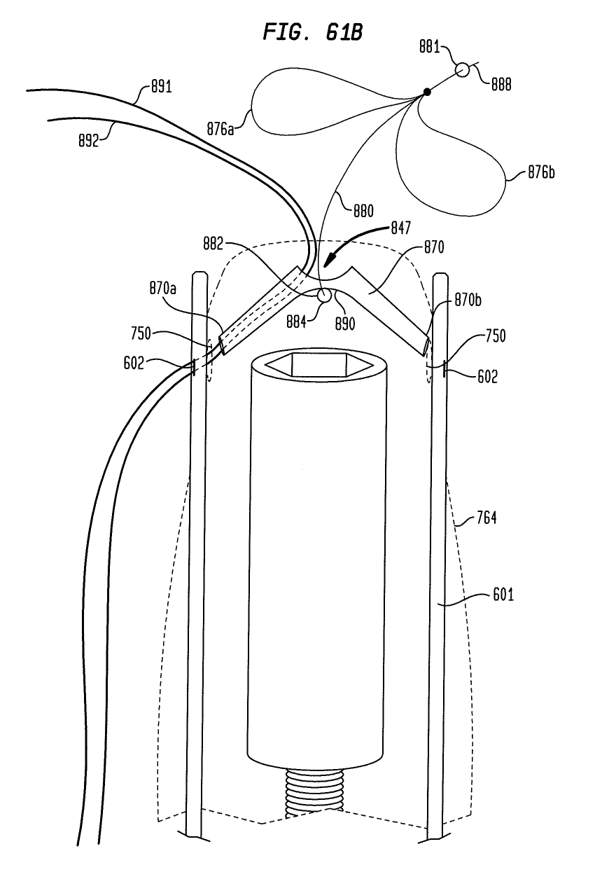

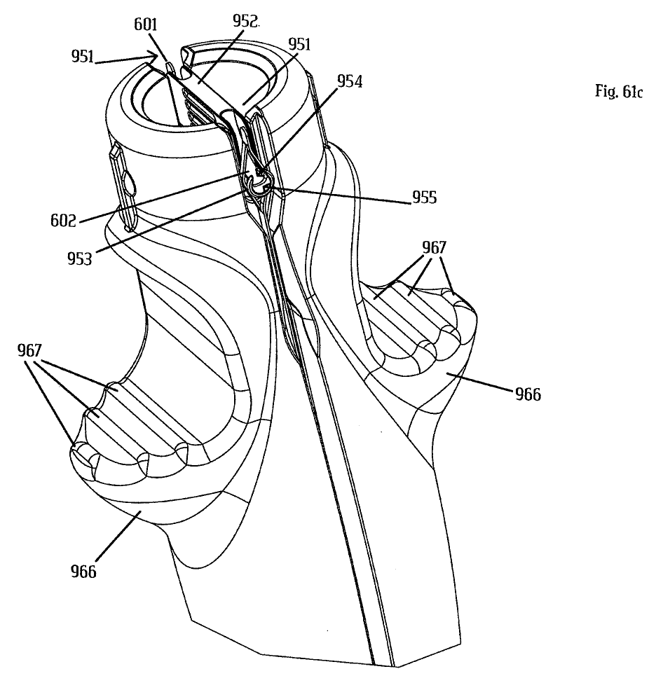

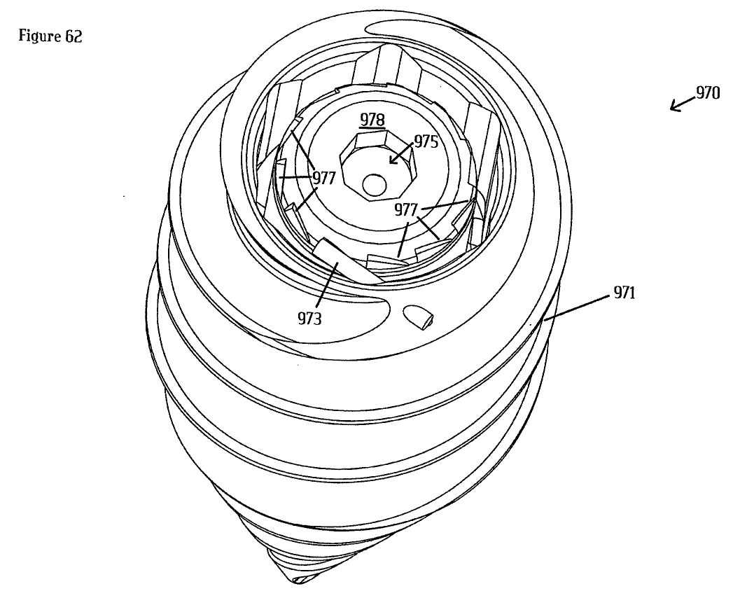

FIGS. 61A and 61B illustrate another alternate embodiment of the proximal portion of the implantation tool that offers another way to release the suture shuttle from the apertures. This embodiment is substantially similar to the embodiment described above in connection with FIGS. 60A-60C. In this embodiment, slack can be provided in the suture shuttle or purposes of loading it on the apertures and then that slack can be taken up by rotation of the nut essentially in the same way as described in connection with the embodiment of FIGS. 60A-60C. However, the clip bearing the apertures has been replaced with a single tube as described in detail below.

FIG. 61A shows the tool in its pre-surgical condition and FIG. 61B shows the tool in the condition it would appear when the suture shuttle 601 is in the process of being dismounted from the apertures. Most of the components of the tool may remain that same as in the embodiments of FIGS. 57A-57K and/or 60A-60C and such components have been labeled with the same reference numerals in FIGS. 61A and 61B.

With reference first to FIG. 61A, in this embodiment, a hollow tube 870 that runs between the two opposed windows 750 in the handle 764 of the tool serves as both apertures.

The tube comprises a passageway 871 running the entire length of the tube from opening 872 at one end 870a to opening 873 at the other end 870b. Intermediate the two ends of the tube is at least one lateral opening in the side wall of the tube. In the illustrated embodiment the opening comprises a single opening 847 approximately half way between the two ends of the tube. However, this is merely exemplary. In other embodiments, the opening may, for instance, comprise two openings, a first opening near the first end 870a and positioned so that it is inside of the body of the handle 764 and a second opening near the second end 870b of the tube 870 and positioned so that it also is inside of the body of the handle 764 when the tube 870 is mounted on the handle of the tool.

In any event, the lateral opening 847 serves several functions. First, two wire loops 876a, 876b that will be used to pull suture(s)-to-be-shuttled through the apertures are pre-surgically positioned in the tube 870 as shown in FIG. 60A. Specifically, each wire loop extends between the lateral opening 847 and one of the openings 872 or 873 in the ends of the tube. Thus, in essence, the portion 870c of the tube 870 between lateral opening 847 and end 870a may be considered to be one of the apertures and the portion 870d of the tube 870 between lateral opening 847 and end opening 873 may be considered to be the other of the apertures. In the pre-surgical condition (FIG. 60A), the wire loops 876 extend beyond the openings 872, 873 in the ends of the tube 870 so that suture(s)-to-be-shuttled may be passed through them for purposes of loading the sutures into the slits 602 of the suture shuttle 601 and apertures 870c, 870d in the same manner as described in connection with the embodiment of FIGS. 60A-60C, for instance.

The lateral opening 847also serves the function of providing a weakened section 890 of the tube 870 about which the tube can fold or bend upon application of sufficient force so that the tube 870 may be removed from tool handle 764 thereby releasing the suture shuttle 601 from the apertures 870c, 870d, as will be described in more detail below. In this embodiment, a sufficient portion of the side wall of the tube is removed to from the lateral opening 847 so that the tube 870 will bend upon an application of a predetermined lateral force of about 2 pounds. This predetermined bending force may be designed into the tube by appropriate selection of wall thickness of the tube, amount of material removed to form the opening 847, and/or thinning the wall of the remaining portion or the tube adjacent the opening 847 (such as by etching a groove therein).

It should be apparent that, pre-surgically, the tube 870 is trapped in the handle 764 by virtue of the tube being longer than the distance between the two windows 750 in the handle. It also should be apparent that the tube 870 can be acted upon by the nut 566 to push the tube proximally within the windows 750 to take up slack in the suture shuttle 601 essentially exactly as described above with respect to the apertures 711 in the embodiment of FIGS. 60A-60C.

In one embodiment, the wire loops 872 and 873 are attached to another wire 880 that is disposed in the tube 870 in the pre-surgical condition. In this embodiment, the wire loops 872, 873 are attached to the wire 880 about half way between the two ends 881, 882 of the wire 880. The proximal end 881 of the wire 880 extends out of the lateral opening 847 in the tube so that a surgeon can grasp it and pull on it to draw the wire loops 872, 873 through the aperture portions 870c and 870d of the tube and out of the lateral opening 847 carrying the suture(s)-to-be-shuttled with them, thereby loading the suture(s)-to-be-shuttled onto a slit 602 in the suture shuttle 601 essentially as previously described in connection with various above-discussed embodiments. A ball 888 or other device may be attached to the proximal end 881 of the wire 880 to facilitate grasping by the surgeon.

The distal end 882 of the wire 880 is designed so that the distal end of the wire 880 is attached to the tube 870 and cannot be being removed from the tube 870. This attachment may take a variety of forms. In one embodiment, the distal and 882 of the wire 880 may be welded, adhered, or otherwise attached to the tube 870. In the illustrated embodiment, however, the distal end 882 of the wire 880 actually passes through a hole 885 in the tube 870 that is positioned substantially opposite to the lateral opening 847 and has a blocking member 884, such as a ball or pin attached to distal end 882 that cannot pass through the hole 885.

FIG. 61B shows the tool handle after two sutures-to-be-shuttled 891, 892 have been loaded through the aperture 870c and slit 602. Thus, after the wire loops and suture(s)-to-be-shuttled have been loaded through the aperture portion 870c or 870d and out of the lateral opening 847, the surgeon continues to pull on the proximal end 881 of the wire 880 with enough force to bring the wire 880 to tension (because the distal end 882 of the wire 880 is attached to the tube 870) and bend the tube 870 at the weakened section 890 adjacent lateral opening 847. When the tube 870 bends at section 888, the two ends 870a, 870b of the tube move laterally toward each other, thereby pulling the ends 870a, 870b free of the windows 750 in the tool handle 764 (and, hence, also free of the slits 602 in the suture shuttle 601). Hence, the suture shuttle 601 is released from the tool handle 764 and, simultaneously, the tube 870 is removed from the tool handle 764.

Although not illustrated in the Figures, as the tube 870 is further removed from the handle 764, the suture(s)-to-be-shuttled 891, 892 continue to simply slide through the tube 870a or 870b and become free of the tube 870 (while remaining loaded in the slit 602 of the suture shuttle 601, which closes once the tube 870 is pulled free of the slit 602).

The wire loops 876a, 876b should be attached to the wire 880 at a point 889 along the wire 880 so that there is enough of wire 880 distal of the attachment point 889 to allow the wire loops 876a, 876b attached to the wire 880 at point 889 to be pulled completely through and out of lateral opening 847 before the wire 880 is fully extended under tension. This is because the wire loops 876a, 876b and suture(s)-to-be-shuttled 891, 892 should be completely loaded through the aperture 870c or 870d before the tube is bent to release the shuttle 601 from the aperture.

FIG. 61C shows yet another embodiment of the proximal portion of the implantation tool that offers another way to release the suture shuttle from the apertures. In this embodiment, two slots 951 open to the proximal end of the tool are positioned diametrically opposite to each other with an insert 952 running laterally between and in a friction fit with the slots 951. The insert 952 has a channel structure 953 extending laterally from each side through the slots 951 in the handle (only one channel structure 953 can be seen in the view of FIG. 61C). The channel structure has a side opening 954 at its top, thus defining an open channel 955 in the tube (rather than a radially closed bore). The outer surface of the channel structures 953 are used to hold the slits 602 in the suture shuttle 601 open as substantially described above in connection with the embodiments of FIGS. 57A-61B. With this embodiment, the surgeon will manually insert the sutures into the channels 955 and, thus, through the open slit 602 of the suture shuttle 601. Next the suture shuttle can be released from the channel structure 953 by simply pulling up and/or radially outward of the tool on the suture ends. This will cause the sutures to move through the opening 954 in cylinder 953 of insert 952 and bear on the edge of opening 602 in suture shuttle 601 and pull the suture shuttle 601 off of the channel structure 953. The other side of the suture shuttle may be released essentially as described previously in connection with the embodiment of FIGS. 57A-57J. Finally, the surgeon can pull the insert 952 out of the handle through the open ends of the slots 951, such as by grasping it with a hemostat or the like and pulling upward to provide access to the nut inside the handle.

Also, note another feature of this embodiment is a variation of the thumb rests 966 (as compared to the embodiment of FIGS. 57A-57K, for instance). Thumb rests 966 are larger and present a larger, more laterally oriented top surface with gripping ridges 967 in order to offer the surgeon an enhanced surface for resting his or her thumbs when tensioning the suture just prior to locking the sutures in the anchor, as previously described

Tenth Set of Exemplary Embodiments

In surgery, it is possible that the surgeon may find that the sutures have been locked in the eyelet with less tension on the tissue than desired. For example, it is often the case in a double-row repair such as described above in connection with FIGS. 34 and 35, that the surgeon locks the sutures in a first anchor and then subsequently locks the sutures in a second anchor with more tension than the sutures were locked in the first anchor, causing the sutures locked in the first anchor to become less tensioned.

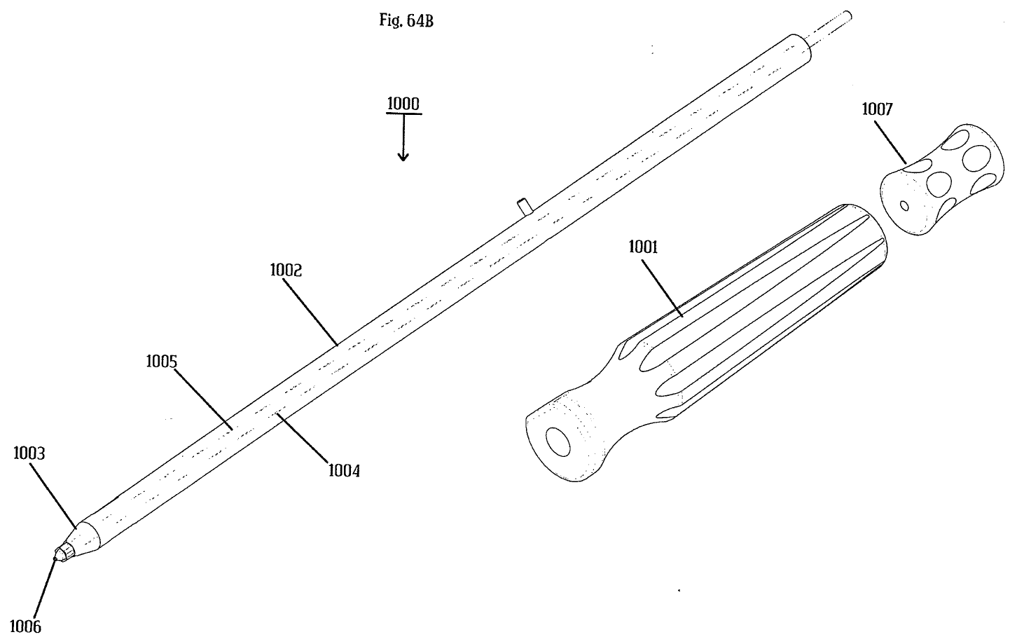

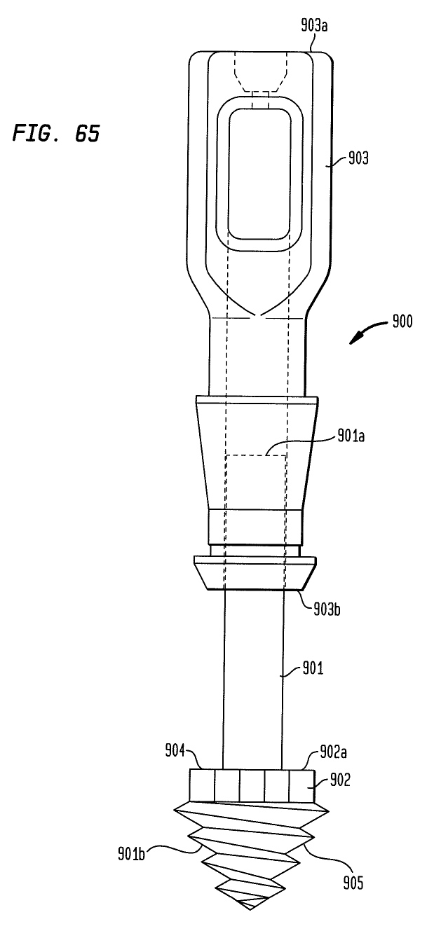

The tension on the sutures may be increased by screwing the anchor further down into the bone, such as by using the tool described hereinbelow in connection with FIGS. 64A and 64B. However, other mechanisms also may be provided for enhancing the ability to tension after implantation.

FIG. 62 illustrates an embodiment of the bone anchor with a ratchet and pawl mechanism between the eyelet pin and the anchor main body that provides a mechanism by which sutures already locked in the eyelet of the eyelet pin may be tightened even further. Particularly, FIG. 62 is a top plan view of a bone anchor that will permit the surgeon to twist the eyelet pin around its longitudinal axis after the eyelet pin has been pushed down into the closed position locking the sutures in the eyelet. The twisting will wrap the sutures around the eyelet pin in order to take up slack or increase tension on the sutures.

The bone anchor 970 illustrated in FIG. 62 is essentially identical to the bone anchor described above in connection with FIGS. 52 to 56C, except for the addition of the ratchet and pawl system as described below. However, this is merely exemplary insofar as this concept of a locking, rotatable eyelet pin can be applied to any of the anchors described in this specification and, in fact, to other anchors. Particularly, a series of ratchet cogs 977 may be disposed on the outer radial wall of the eyelet pin 978 of the bone anchor main body 971. Further, a pawl in the form of pin 973 may be included in the radial wall 972 of the internal bore of the anchor main body 971. The mechanism will allow the eyelet pin 978 to be rotated in one direction and prevent it from rotating in the opposite direction. The ratchet pawl may rely on the inherent resilience of their specific design and/or material from which they are made to permit the cogs 977 to clear the pin 973 (in one direction). Alternately, the pin may be spring loaded to allow the pawl to clear the ratchet cogs (in one direction).

With such a mechanism, the proximal bore 975 of the eyelet pin 978 may be contoured to mate with the head of a torquing tool that may be inserted into the proximal bore 975 of the eyelet pin 978 in order to turn it so as to wrap the sutures around the eyelet pin to increase the tension on the sutures. As mentioned above, this may be done with the eyelet pin 1104 in the closed position. However, it also may be performed with the eyelet pin still in the open position to set the desired tension before locking. For instance, with the surgeon manually holding tension on the sutures, he or she may twist the eyelet pin 978 in order to wrap the sutures around the eyelet pin and increase the tension on the sutures, and then, subsequently, drive the eyelet pin into the closed position, locking the sutures in the eyelet.

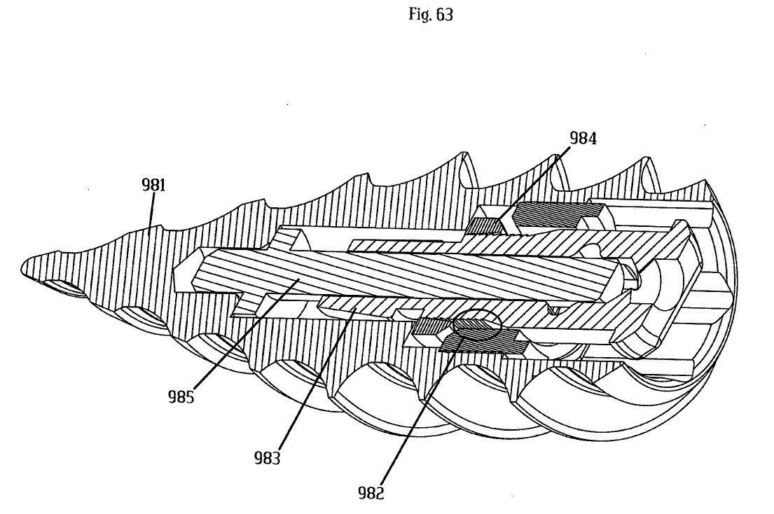

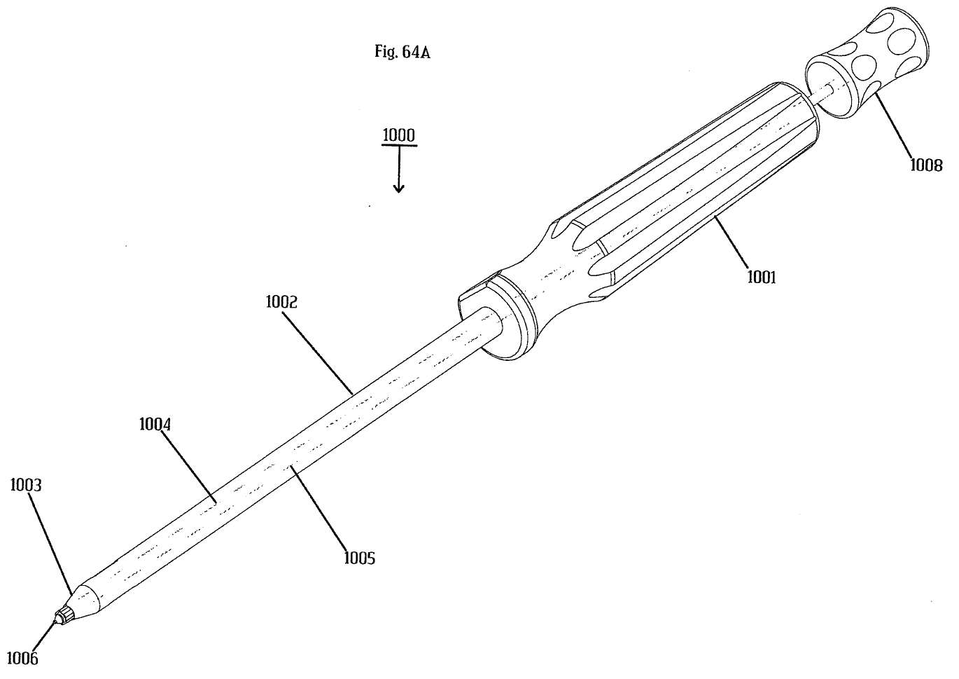

FIG. 63 illustrates (in perspective cross-section) yet another possible embodiment providing a means by which suture tension may be increased after locking. This embodiment provides a mechanism by which the eyelet pin 983 can be driven further down into the anchor body 981 even after the eyelet pin has been deployed into the closed position. For instance, as illustrated in FIG. 63, the second ramp 407 on the body of the eyelet pin of FIGS. 36-41 may instead be replaced with a vertically oriented ratchet 982 (essentially a plurality of mini ramps) Particularly, as discussed above in connection with the embodiment of FIGS. 36-41, the first ramp 983 (406 in the embodiment of FIGS. 36-41) acts in conjunction with the C ring 984 (404 in the embodiment of FIGS. 36-41) to keep the eyelet pin from falling out of the anchor body when in the open position. In the embodiment of FIGS. 36-41, the second ramp 407 acts in conjunction with the C ring to hold the eyelet pin in the closed position after deployment.

In this embodiment, the ratchet 982 essentially is a plurality of mini ramps to permit the eyelet pin to be driven into a plurality of different closed positions, each one successively deeper in the anchor main body 981. Thus, the surgeon can lock the sutures in the eyelet by driving the eyelet pin 983 down so that only the lowest mini ramp ratchets past the C ring 984. Then, if it is later desired to increase the tension on the suture locked in the eyelet, the surgeon can return to the anchor and drive the eyelet pin further down over the central pin. Any reasonable impactor-type tool, such as the impactor tool described hereinabove in connection with FIGS. 46-48, may be used to drive the eyelet pin further down as described so that further ones of the mini ramps pass the C ring.

Exemplary Embodiment of a Redeployment/Adjustment Tool

As previously mentioned, the bone anchor of the present invention is adjustable or redeployable after implantation, if necessary. FIG. 64A is a perspective view of an exemplary redeployment/adjustment tool 1000 for such purposes and FIG. 64B is a cross-sectional side view of the distal end of the tool. This tool 1000 is particularly adapted to work with the bone anchor 581 illustrated in FIGS. 56A-56C. However, other designs adapted to work with the same or different anchors are possible. This particular exemplary tool comprises a removable handle 1001, a shaft 1002 extending from the handle 1001, and a shaped head 1003 at the distal end of the shaft.

The handle 1001, shaft 1002, and associated head 1003 can be used to adjust or remove an implanted bone anchor. Particularly, if a bone anchor needs to be adjusted or removed after implantation, the tool 1000 may be inserted to engage the bone anchor 581 with the head 1003 of the tool. More particularly, the head 1003 of the tool 1000 may be shaped essentially identical to the shaped head 563c of the shaft 563 of the implantation tool 561 described above in connection with FIGS. 54A-56C. That is, it has a head shaped to match the pattern 584 of the proximal end of the internal bore 560 of the main body 580 of the anchor 581. Preferably, the head 1003 is shaped and sized to form a friction fit between the wall of the inner-bore 560 of the bone anchor main body 580 and the outer radial wall of the eyelet pin 521 and/or friction ring 552. If the eyelet pin has an overhang such as the overhang 524 of the eyelet pin 521 of the embodiment of FIG. 56A, the tool head 1003 would need to accommodate such overhang. The friction fit should be strong enough to permit the bone anchor to be lifted out of the bone after it has been unscrewed from bone (in the case of redeployment), yet weak enough that, if the bone screw 581 remains implanted in the bone, pulling up on the tool 1000 will cause the head 1003 to slip out of the interference fit without causing the bone screw to tear out of the bone or otherwise disturb the bone in which it is implanted. Hence, using the redeployment/adjustment tool 1000, the surgeon can (1) screw the bone screw further into bone, (2) screw it partially out of the bone, or (3) screw it entirely out of the bone and redeploy it in another location or remove it from the patient's body, as needed.

Since, as previously described, the central pin and eyelet pin combination is freely rotatable within the anchor body, the anchor may be further screwed into the bone even after sutures are positioned in the eyelet without problem. While the suture may become wrapped around the adjustment tool during screwing, once the adjustment tool is removed, the central pin and eyelet pin combination will simply rotate within the anchor body back to a rotational orientation in which the eyelet passage aligns with the direction from which the sutures emanate.

The tool 1000 also may be designed to serve double duty as the tool for turning the eyelet pin in the above-described embodiment of FIG. 62. For instance, a bore 1004 may be provided through the handle 1001, shaft 1002, and head 1003 of the tool with a rod 1005 disposed in the bore 1004 that is rotatable and translatable therein. This internal rod 1005 bears a second head 1006 shaped to engage the proximal bore 975 in the eyelet pin 978 of FIG. 62 (which has a mating pattern to allow the eyelet pin to be twisted by turning the rod 1005 of the tool 1000). There are any number of designs that would allow a surgeon to manipulate rod 1005 inside of bore 1004 from the proximal end of the tool 1000. FIGS. 64A and 64B illustrate one such embodiment. In this embodiment, internal rod 1005 runs completely through rod 1002 and first handle 1001 and extends from the proximal end of tool 1000 so that a second handle 1007 can be placed on the proximal end of rod 1005. The second handle 1007 is engagable with the proximal end of rod 1005 so that the second handle 1007 can be manipulated to both (1) advance the internal rod 1005 distally relative to external shaft 1002 (and head 1003) so that it can engage the proximal bore 975 of the eyelet pin 978 (without also causing the external head 1003 to engage the pattern 584 in the proximal end of the internal bore 560 of the main body 580 of the anchor 581) and (2) twist the internal rod 1005 and its head 1006 independently of the shaft 1002 and its head 1003 to allow the surgeon to rotate the eyelet pin 978 to cause the suture to wrap around it as previously described.

When the tool 1000 is used in situations where it is not necessary, possible, or desired to wrap the sutures around the eyelet pin, the entire inner structure (rod 1005, head 1006, and handle 1008) can be omitted from the tool structure (or at least removed from the tool prior to use).

On the other hand, a head such as head 1006 for engaging a proximal bore in the eyelet pin may be useful even during redeployment, namely, as a guide for guiding the primary tool head 1003 into engagement with the anchor during redeployment/adjustment. Thus, the head 1006 may be spring-loaded on the rod 1005 to help in guiding the primary head into the anchor body. Alternately, in embodiments of the tool 1000 not adapted to twist the eyelet pin, a spring-loaded tip may be provided extending from the end of the shaft 1002 inside of and through head 1003.

Eleventh Set of Exemplary Embodiments

FIG. 65 illustrates another alternative embodiment of the present invention. In this embodiment, the entire implant comprises merely a central pin 901 and an eyelet pin 903. There is no separate anchor body or related accoutrements (such as the C-ring or retaining ring). The central pin and eyelet pin similar to the central pin and eyelet pin of the embodiment of FIGS. 52A-56C, but this is merely exemplary. Specific implementations in accordance with this embodiment of the invention also may be made using the central pin and eyelet pin of FIGS. 36-41 (or other configurations).

In any event, in this embodiment, the distal end 901b of the central pin 901 itself bears threads 905. Therefore eliminating any need for a separate anchor main body for purposes of attaching the anchor 900 to bone. The outer periphery of the shelf 902 in the central pin 901 may bear formations 904 to mate with a torquing tool having mating internal formations so that the anchor 900 may be screwed into bone. In fact, a tool for implanting this particular device may be quite similar to the tool 561 discussed above in connection with the embodiments of FIGS. 52A-57K. Particularly, in one embodiment, the tool may be frangibly connected to the shelf 902 of the central pin 901 much in the same way that the tool 561 was frangibly attached to the retaining ring 541 in the embodiments of FIGS. 52A-57K so that the tool and central pin 901 can be rotated about their longitudinal axes to screw the anchor 900 into bone without loading the frangible portions. Also similarly, the eyelet pin 903 can be driven down over the central pin 901 until the distal end 903b of the eyelet pin hits the top surface 902a of the shelf 902 using essentially the same structure for achieving this as is found in the tool 561 of the embodiments of FIGS. 52A-57K. Specifically, just as in that embodiment, as the rod in the tool pushes against the top 903b of the eyelet pin 903, the eyelet pin 903 is forced down over the central pin 901 until it hits the shelf 902. Thereafter, continued pushing of the rod will force the hollow shaft of the tool upward relative to the rod and eyelet pin 903, thereby breaking the frangible portions and releasing the tool from the anchor 900.

Not only may the central pin and eyelet pin concept of the present invention be used (1) with anchor main bodies, as described in connection with FIG. 36-57K or (2) independently of any separate anchor body, as described hereinabove connection with FIG. 65, but it also may be incorporated into many other implants or bodies.

Furthermore, the anchors described hereinabove have been discussed primarily in connection with use in connection tissue to bone by attaching sutures to the tissue and then attaching those sutures to the anchors. However, in other applications, the anchors may be used to attach any elongate member, including elongate tissue, directly to bone by passing the tissue itself directly through the eyelets. Ligaments and tendons, for instance, can be passed directly through the eyelet of one of the aforedescribed bone anchors instead of a suture attached to the ligament or tendon.

### CONCLUSION

As mentioned earlier, the exact configurations of the bone anchor devices are greatly variable, particularly within the parameters hereinabove described. Individual devices thus can be associated with particular predetermined features that will render them most effective for performing specific procedures. Also it should be noted that many of the features described in connection with individual embodiments of the present invention may be substituted into one or more of the other embodiments described herein, there being no limitation other than logic and physical limitations as to how the various features can be mixed and matched in a single device. The same is true for the surgical procedures disclosed herein, i.e., certain aspects of certain of the described surgical procedure embodiments may be used in other described surgical procedure embodiments described herein and/or may be performed in connection with other embodiments of the bone anchor devices and/or time fastener devices than those used in the exemplary embodiments described herein.

The procedures and medical devices as described can be altered in various further ways while still accomplishing the same results and the invention also covers such variations in the procedure.

It is submitted that, with the use of the present invention, the arthroscopic rotator cuff repair procedure is significantly facilitated by the use of the bone anchor device and/or the tissue fastener device of the present invention.

It must be understood in the above regard that one of the biggest challenges in arthroscopic surgery is knot tying. It is technically challenging and, insofar as the use of the bone anchor devices and/or the tissue fastening devices of the invention facilitate knotless suture fixation, the challenges associated with knot tying are largely overcome.

It must also be understood in the above regard that another challenge in arthroscopic surgery is suture management. It is technically challenging and, insofar as the use of the medical device of the invention facilitates effective suture management and loading of the suture anchor, the challenges associated with suture management are largely overcome.

Although other knotless fixation devices are already known, some of these require an anchor body to which a suture must be anchored to be located in a pilot hole. It is technically challenging to place an anchor body into the pilot hole, particularly because the hole often bleeds, obscuring the hole and, even if the hole does not bleed, recreating the exact angle that was used during the creation of the pilot hole is sometimes difficult. Placement of cannulas directly over a pilot hole also may create a suction effect dragging soft tissue over the hole, further obscuring it. It is thus often time-consuming and frustrating to locate the hole and correctly locate the bone anchor device in the hole. Incorrect angular location of an anchor device in a hole may occur from the precise angle of insertion necessary for good bone purchase and this may result in failure of some of the known knotless fixation devices. The procedures associated with the self drilling and self tapping bone anchor devices of the present invention as above described alleviate the problem of finding a pilot hole for a bone anchor device. Insofar as the use of other known knotless fixation devices and generally anchor devices may be associated also with various other problems and difficulties, either generally or specifically in relation to specific devices, the use of the medical device of the invention may serve also to at least alleviate these problems and difficulties.

It is also known that all presently available anchor designs are “buried” below the bone. This is done to prevent impingement of the head of the device with surrounding anatomy. Although the medical device of the invention may use a body with either no head or a lower profile head that allows the body to be buried below the bone, there are distinct advantages to using an anchor main body having a head that remains accessible externally of the humerus. As such, the anchor main body can be easily unscrewed from the humerus. With respect to some embodiments described herein, it is also possible to pull the eyelet pin from its anchor main body. The above may be necessary where a repair has failed and/or is not satisfactory and needs to be removed, where inadvertent suture dislodgement from the anchor device has occurred where irreversible tanglement of sutures has occurred, and/or where a suture knot comes loose. It is envisaged in this regard that bone anchor devices in accordance with the invention may be provided with anchor main bodies of larger diameter for placement in original holes formed by removed anchor main bodies to provide for optimal purchase strength of the device to bone. The use of the bone anchor device of the invention, therefore, reduces or eliminates the need, in the circumstances described above, for placing additional anchors within the limited space available for a repair, additional bone anchors may induce the risks of confluence of anchor holes, bone fracture and/or anchor pull-out. It must also be understood in relation to the use of known anchor devices, that at times the devices can be removed only by coring techniques that are cumbersome and time consuming and that often lead to significant bone loss that requires bone grafting. Bone grafting in itself may be associated with problems, thus rendering the use of the medical device of the invention significantly more appropriate in relation to many different procedures, when compared with the use of known anchoring techniques and anchoring devices, even known knotless fixation devices.

It is thus submitted that the known problems associated with the tying of sutures, the management of sutures and also the anchoring of sutures to the humerus, are largely alleviated, the same applying also in relation to other procedures with which the medical device of the invention can be conveniently used, either arthroscopically, or otherwise.

Having thus described a few particular embodiments of the invention, various alterations, modifications, and improvements will readily occur to those skilled in the art. Such alterations, modifications, and improvements as are made obvious by this disclosure are intended to be part of this description though not expressly stated herein, and are intended to be within the spirit and scope of the invention. Accordingly, the foregoing description is by way of example only, and not limiting. The invention is limited only as defined in the following claims and equivalents thereto.

1. A system for delivering a bone anchor device for anchoring an elongate member to a bone comprising:
a bone anchor device including;
a main anchor body defining a longitudinal axis, a receiving formation, and a formation for accepting a driving tool for driving the anchor main body into a bone;
an eyelet pin defining a longitudinal axis and an eyelet substantially transverse to the said axis through which an elongate member can be passed;
wherein the eyelet pin is longitudinally insertable in the receiving formation from an open position in which an elongate member may pass freely through the eyelet to a closed position in which an elongate member passing through the passage in the eyelet pin would be securely held to the eyelet pin; and
a driving tool having a distal end and a proximal end, the driving tool including;
a shaft having a bore therein extending from an opening in the proximal end of the tool to an opening in the distal end of the tool;
a rod disposed within the bore of the shaft having a distal end and a proximal end, the distal end of the rod abutting the eyelet pin of the bone anchor;
a retaining ring frangibly attached to the distal end of the shaft, the retaining ring also attached to the main anchor body;
the rod being coupled to the shaft via a screw thread that permits the rod to be moved longitudinally distally relative to the shaft by rotation of the threaded engagement relative to the shaft, thereby forcing the eyelet pin into the anchor main body to the closed position.

2. The system of claim 1 further comprising a nut disposed between the shaft and the rod, the nut bearing the screw threads and being threadedly engaged to the shaft such that rotation of the nut relative to the shaft will cause the nut to move longitudinally relative to the shaft, and the nut abutting the proximal end of the rod such that longitudinal distal movement of the nut relative to the shaft results in longitudinal distal movement of the rod relative to the shaft.

3. The system of claim 1 wherein the eyelet pin comprises a shelf positioned on the eyelet pin relative to the receiving formation in the anchor main body so that, when the eyelet pin is in the open position it may move longitudinally distally within the receiving formation to the closed position until the shelf meets with a surface of the receiving formation that will prevent further distal longitudinal movement of the eyelet pin relative to the anchor main body, whereby further distal advancement of the rod relative to the shaft causes the shaft to start moving proximally relative to the retaining ring and anchor main body.

4. The system of claim 3 wherein the retaining ring is fixedly attached to the anchor main body via friction resulting from an interference fit between the retaining ring and the anchor main body and the force to overcome the frictional engagement to cause the retaining ring to move relative to the anchor main body is greater than the force to break the frangible connection between the retaining ring and the shaft.

5. The system of claim 4 wherein the tool further comprises a handle fixedly attached to the shaft and wherein the handle comprises:
a proximal end and a distal end;
a longitudinal bore in the proximal surface of the handle adapted to receive the nut and having a length greater than a length of the nut;
wherein the nut is disposed in the proximal bore of the handle and can travel longitudinally in the proximal bore.

6. The system of claim 4 wherein the nut comprises a proximal cavity having a configuration for accepting a torquing tool for turning the nut about its longitudinal axis relative to the shaft.

7. The system of claim 1 wherein the anchor body further comprises a central pin disposed in the receiving formation and having a longitudinal axis parallel to the longitudinal axis of the anchor body and eyelet pin, the central pin having a proximal end and a distal end,
wherein the eyelet pin has a proximal end and a distal end and includes a distal bore running between and open to each of the transverse passage and the distal end of the eyelet pin and wherein the distal longitudinal bore of the eyelet pin forms an interference fit with the central pin and wherein an elongate member passing through the passage is securely held in the eyelet pin by virtue of being trapped in the passage by the central pin when the eyelet pin is in the closed position.

8. A bone anchor device comprising:
a central pin having a longitudinal axis, the central pin having a proximal end and a distal end and having an external formation for engaging the central pin to a bone; and
an eyelet pin defining a longitudinal axis and having a passage substantially transverse to the longitudinal axis, through which an elongate member can be passed;
wherein the eyelet pin has a proximal end and a distal end and includes a distal longitudinal bore running between and open to each of the transverse passage and the distal end of the eyelet pin and wherein the distal longitudinal bore of the eyelet pin forms an interference fit with the central pin;
wherein the eyelet pin is longitudinally insertable over the central pin into a closed position in which an elongate member passing through the passage in the eyelet pin would be securely held in the transverse passage of the eyelet pin by the central pin.

9. The bone anchor device of claim 8 wherein the bone anchor device has an open position in which an elongate member would be freely slidable through the passage and wherein, in the open position, the central pin does not intrude into the transverse passage and wherein the eyelet pin is translatable relative to the central pin along their respective longitudinal axes from the open position to the closed position, in which closed position the proximal end of the central pin extends into the transverse passage so as to trap an elongate member passing through the transverse passage between the proximal end of the central pin and a wall of the transverse passage.

10. The bone anchor device of claim 9 wherein the transverse passage has a top wall and a clearance between the proximal end of the central pin and the top wall of the transverse passage when the device is in the closed position is less than a diameter of an elongate member passing through the transverse passage.

11. A suture shuttle for shuttling a suture through an eyelet of a bone anchor during a surgical procedure, the suture shuttle comprising:
a ribbon of resilient material having a first end and a second end; and
at least one opening in the shuttle.

12. The suture shuttle of claim 11 wherein the opening comprises at least one slit in the shuttle adjacent the first end of the shuttle, the at least one slit including at least a portion defining an opening smaller that a diameter of a suture to be shuttled using the suture shuttle.

13. The suture shuttle of claim 11 wherein the resilient material is metal.

14. The suture shuttle of claim 13 wherein the metal is an alloy of nickel and titanium.

15. The suture shuttle of claim 11 wherein the slits are laser etched slit.

16. The suture shuttle of claim 11 wherein each the at least one opening has an open position, in which a suture may slide through the opening, and a closed position, in which a suture passing through the opening would be longitudinally fixed within the opening, wherein the resilient material of the ribbon biases the opening into the closed position.

17. The suture shuttle of claim 12 wherein the resilient material of the ribbon biases the at least one slit into a first condition in which an opening defined by the slit is a first, smaller size and wherein the resilient material of the ribbon may be resiliently flexed by application of a force so as to place the at least one slit in a second condition in which an opening defined by the slit is a second, larger size, where, upon removal of the force, the slit returns to the first size.

18. A system for loading a suture through an eyelet in a suture anchor, comprising:
a suture anchor including an eyelet for accepting sutures therethrough;
a tool for implanting the suture anchor, the tool having a distal end and a proximal end, the suture anchor attached to the distal end of the tool, a handle attached to the proximal end of the tool and a shaft extending between the proximal end and the distal end;
a suture shuttle comprising a ribbon of flexible material having a first and a second end and at least one opening for accepting a suture therethrough in a longitudinally fixed manner; and
at least one aperture disposed on the handle, said aperture comprising an outer surface and an inner opening, the outer surface adapted to accept the opening of the suture shuttle thereover.

19. The system of claim 18 wherein the at least one opening on the suture shuttle comprises first and second openings and the at least one aperture disposed on the handle comprises first and second apertures on the handle and wherein the suture shuttle is mounted to the tool with the first opening mounted over the outer surface of the first aperture, the second opening mounted over the outer surface of the second aperture, and a longitudinal segment of the ribbon between the first and second openings extending down one side of the shaft, through the eyelet, and up the other side of the shaft.

20. The system of claim 19 wherein the outer surfaces of the apertures are of a size and shape larger than the openings when the openings are in an unstressed condition and wherein the openings are mounted to the apertures by resilient deformation of the opening into a stressed condition.

21. The system of claim 18 further comprising a wire loop disposed through the inner opening of at least one of the apertures and extending from both ends of at least one aperture.

22. The system of claim 21 wherein the wire loop extends through both the inner opening of both the first aperture and the second aperture.

23. The system of claim 21 further comprising:
a cap adapted to be removably mounted to the handle, wherein the wire loop is attached to the cap.

24. A system for loading a suture through an eyelet in a bone anchor, comprising:
a bone anchor including an eyelet for accepting sutures therethrough;
a tool for implanting the bone anchor, the tool having a distal end and a proximal end, the bone anchor attached to the distal end of the tool, a handle attached to the proximal end of the tool and a shaft extending between the proximal end and the distal end;
a suture shuttle comprising a ribbon of resilient material having a first and a second end and first and second openings for accepting a suture therethrough in a longitudinally fixed manner;
first and second openings disposed in the handle;
a tube extending between and through the first and second openings in the handle, the tube having first and second longitudinal ends, the tube defining a passageway therein extending between a first opening in the first longitudinal end of the tube and a second opening in the second longitudinal end of the tube, a third opening intermediate the first and second ends of the tube and a weakened segment about which the tube will deform when subjected to a transverse force;
a suture loading mechanism comprising at least one wire loop extending through the tube between the third opening and the first opening and including a portion extending out of the first opening, the wire loop being attached to the tube in a manner such that the wire loop may be substantially removed from the tube through the third opening while still attached to the tube, and may be further pulled to apply a transverse force to the tube to cause it to bend about the weakened segment and be removed from the handle in a direction transverse to the tube.

25. The system of claim 24 wherein the suture loading mechanism further comprises a wire segment, the wire segment comprising a first end attached to the tube and a second, free end, and wherein the wire loop is attached to the wire segment intermediate the first and second ends thereof.

26. The system of claim 25 wherein the suture loading mechanism further comprises a second wire loop attached to the sire segment intermediate the first and second ends thereof, the second wire loop extending between the third opening and the second opening and including a portion extending out of the second opening.

27. The system of claim 24 wherein:
the bone anchor comprises:
a main anchor body defining a longitudinal axis, a receiving formation, and a formation for accepting a driving tool for driving the anchor main body into a bone;
an eyelet pin defining a longitudinal axis and bearing the eyelet, wherein the eyelet pin is longitudinally insertable in the receiving formation from an open position in which a suture may pass freely through the eyelet to a closed position in which a suture passing through the passage in the eyelet pin would be securely held in the eyelet;
the driving tool shaft has a bore therein extending from an opening in the proximal end of the tool to an opening in the distal end of the tool, the distal end of the shaft frangibly attached to the main anchor body;
a rod disposed within the bore of the shaft having a distal end and a proximal end, the distal end of the rod abutting the eyelet pin of the bone anchor;
the rod being coupled to the shaft via a screw thread that permits the rod to be moved longitudinally distally relative to the shaft by rotation of the threaded engagement relative to the shaft, thereby forcing the eyelet pin into the anchor main body into the closed position;
a nut disposed between the shaft and the rod, the nut bearing screw threads and being threadedly engaged to the shaft such that rotation of the nut relative to the shaft will cause the shaft to move longitudinally relative to the nut, and the nut abutting the proximal end of the rod such that longitudinal proximal movement of the nut relative to the shaft results in longitudinal proximal movement of the shaft relative to the rod;
wherein the openings in the handle comprise slots within which the first and second ends of the tube can slide in the longitudinal direction of the shaft and wherein the nut abuts the tube to restrict distal movement of the tube within the slot and tension in the ribbon restricts proximal movement of the tube within the slot.

28. A system for loading a suture through an eyelet in a suture anchor, comprising:
a suture anchor including an eyelet for accepting sutures therethrough;
a tool for implanting the suture anchor, the tool having a distal end and a proximal end, the suture anchor attached to the distal end of the tool, a handle attached to the proximal end of the tool and a shaft extending longitudinally between the proximal end and the distal end;
a suture shuttle comprising a ribbon of flexible material having a first longitudinal end and a second longitudinal end and at least one opening for accepting a suture therethrough in a longitudinally fixed manner, the suture shuttle mounted to the tool with a longitudinal segment of the ribbon between the first and second openings extending down one side of the shaft, through the eyelet, and up the other side of the shaft;
wherein the eyelet has a cross section adapted to assure that the ribbon of the suture shuttle passes through the eyelet in a particular orientation.

29. The system of claim 28 wherein the ribbon has a length in the longitudinal direction, a thickness in a direction through the opening in the suture shuttle and a width transverse the length and the thickness, the eyelet has a cross section perpendicular to the direction through the eyelet that is substantially rectangular having a height dimension in a direction parallel to the longitudinal dimension of the shaft and a width dimension in a direction transverse to the height, the width being less than a width of the ribbon over most of the height of the eyelet, and including a ribbon-guiding portion substantially in the middle of the height of the eyelet that is wider than the ribbon, the ribbon-guiding portion having a height substantially less than the width of the ribbon so that the ribbon can fit through the ribbon-guiding portion only with the ribbon's width dimension oriented substantially parallel to the width dimension of the eyelet and wherein a portion of the ribbon within the eyelet is in the ribbon-guiding portion oriented substantially parallel to the width dimension of the eyelet.

30. The system of claim 29 wherein the height dimension of the eyelet is sufficient to accommodate a plurality of folded sutures loaded in the opening in the suture shuttle.

31. A system for loading a suture through an eyelet in a bone anchor, comprising:
a bone anchor including an eyelet for accepting sutures therethrough;
a tool for implanting the bone anchor, the tool having a distal end and a proximal end, the bone anchor attached to the distal end of the tool and having an eyelet for accepting at least one suture therethrough, a shaft extending between the proximal end and the distal end, first and second passages in the proximal end of the tool, and a tube extending between and through the first and second passages in the tool, the tube having an outer surface and defining an open passageway therein extending between a first opening in a first longitudinal end of the tube and a second opening in a second longitudinal end of the tube and a third opening in a transverse side of the tube intermediate the first and second ends of the tube and a weakened segment about which the tube will fold when subjected to a transverse force; and
a suture shuttle comprising a ribbon of flexible material having a first longitudinal end and a second longitudinal end and first and second openings in the ribbon for accepting a suture therethrough in a longitudinally fixed manner;
wherein the suture shuttle is mounted to the tool with the first opening mounted over the first longitudinal end of the tube, the second opening mounted over the second longitudinal end of the tube, and a longitudinal segment of the ribbon between the first and second openings extending down one side of the shaft, through the eyelet, and up the other side of the shaft;
wherein the outer surface of the tube is larger than the openings of the suture shuttle when the openings are in an unstressed condition, and wherein the openings of the suture shuttle are mounted over the tube by resilient deformation of the openings into a stressed condition.

32. The system of claim 31 further comprising a wire having a first end attached to the tube near the weakened segment and a second, free end.

33. The system of claim 32 wherein the weakened segment comprises a lateral portion of the tube longitudinally aligned with the third opening, the weakened segment being weakened by virtue of the third opening.

34. The system of claim 32 wherein the weakened segment is sufficiently weak to permit the tube to be bent by pulling on the wire manually.

35. A system for loading a suture through an eyelet in a bone anchor, comprising:
a bone anchor including an eyelet for accepting sutures therethrough;
a tool for implanting the bone anchor, the tool having a distal end and a proximal end, the bone anchor attached to the distal end of the tool, a handle at the proximal end of the tool, and a shaft extending between the proximal end and the distal end of the tool, at least one channel structure in the handle, the channel structure having an outer surface with an opening defining an open channel in the channel structure; and
a suture shuttle comprising a ribbon of flexible material having a first longitudinal end and a second longitudinal end and at least one opening in the ribbon for accepting a suture therethrough in a longitudinally fixed manner;
wherein the outer surface of the at least one channel structure is larger than the opening in the suture shuttle when the opening is in an unstressed condition and wherein the suture shuttle can be mounted to the tool with the at least one opening mounted over the outer surface of the at least one channel structure by resilient deformation of the opening into a stressed condition and a longitudinal segment of the ribbon extending down one side of the shaft, through the eyelet, and up the other side of the shaft.

36. The system of claim 35 wherein the opening in the channel structure faces proximally of the tool.

37. The system of claim 33 wherein the at least one channel structure on the handle comprises first and second channel structures and the at least one opening on the suture shuttle comprises first and second openings, and further wherein the suture shuttle is mounted to the tool with the first and second openings of the suture shuttle mounted over the outer surfaces of the first and second channel structures and a longitudinal segment of the suture shuttle between the first and second openings of the suture shuttle extends down one side of the shaft, through the eyelet, and up the other side of the shaft.

38. The system of claim 37 wherein the handle of the tool further comprises a slot and an insert disposed in the slot and wherein the first and second channel structures are disposed in the insert.

39. The system of claim 38 wherein the insert is slidably removable from the handle through the slot.

40. The system of claim 39 wherein the slot in the handle is open to a proximal end of the handle and the insert is removable from the handle through the proximal end of the handle.

41. A system for loading a suture through an eyelet in a bone anchor, comprising:
a bone anchor including an eyelet for accepting sutures therethrough;
a tool for implanting the suture anchor, the tool having a distal end and a proximal end, the bone anchor attached to the distal end of the tool, and a shaft extending between the proximal end and the distal end;
at least one passage in the proximal end of the tool;
a clip comprising at least one tube disposed through the at least one passage in the tool; and
a suture shuttle comprising a ribbon of flexible material having a first longitudinal end and a second longitudinal end and at least one opening in the ribbon for accepting a suture therethrough.

42. The system of claim 41 wherein the at least one opening in the suture shuttle is mounted over the at least one tube.

43. The system of claim 42 wherein the clip is manipulable to cause the tube to move relative to the suture shuttle to disengage the suture shuttle from the tube.

44. The system of claim 43 wherein the clip is manipulable to simultaneously cause the tube to slide out of the passage in the tool and cause the suture shuttle to become disengaged from the at least one tube.

45. The system of claim 41 wherein the clip is resilient and is attached to the handle such that the at least one tube is resiliently biased to maintain the at least one tube in a position in which the at least one tube is disposed through the at least one passage in the tool and wherein the clip may be manipulated to overcome the resilient bias to slide the at least one tube out of the at least one passage in the tool.

45. The system of claim 45 wherein the clip further comprises a lever operatively connected to at least one tube and the proximal end of the tool further comprises at least one cam surface and wherein the clip can be manipulated to cause the lever to ride on the cam surface to cause the at least one tube to slide out of the at least one passage in the tool.

47. The system of claim 46 wherein the clip can be manipulated to cause the lever to ride on the cam surface to cause the at least one tube to translate substantially along the longitudinal axis of the at least one tube out of the at least one passage in the tool.

48. The system of claim 47 wherein the manipulation of the clip that causes the lever to ride on the cam surface comprises rotation about a longitudinal axis of the at least one tube.

49. The system of claim 47 wherein the at least one tube comprises first and second coaxial tubes and the at least one passage in the tool comprises first and second aligned passages.

50. The system of claim 43 further comprising a wire loop passing through the at least one tube.

51. A system for loading a suture through an eyelet in a bone anchor, comprising:
a bone anchor including an eyelet for accepting sutures therethrough;
a tool for implanting the suture anchor, the tool having a distal end and a proximal end, the bone anchor attached to the distal end of the tool, a handle attached to the proximal end of the tool and a shaft extending between the proximal end and the distal end;
a suture shuttle comprising a ribbon of flexible material having a first longitudinal end and a second longitudinal end and first and second openings adjacent the first and second ends in the ribbon, respectively, for accepting a suture therethrough;
at least one opening in the handle;
a clip comprising first and second substantially coaxial tubes disposed through the first and second openings in the handle, respectively, and a resilient lever segment extending from and interconnecting the first and second tubes, each tube having first and second longitudinal ends and defining an open passageway therein extending between a first opening in the first end of the tube and a second opening in the second end of the tube, the clip being rotatable, through manipulation of the lever, about an axis defined by the coaxial tubes;
a wire loop disposed through at least one of the tubes and extending from both ends of the at least one tube;
the handle comprising at least one cam surface positioned and shaped to act upon the clip as it is rotated between a first orientation and a second orientation to resiliently deform the clip to cause the first and second tubes to translate relative to each other substantially along an axis defined by the first and second tubes out of the first and second openings in the handle, respectively, whereby the clip can be removed from the handle.

52. The system of claim 51 wherein the first and second openings in the suture shuttle are mounted over the first and second tubes, respectively, and the translation of the first and second tubes causes the first and second tubes to disengage from the first and second openings of the suture shuttle.

53. The system of claim 52 wherein the cam surface acts upon the clip as the clip is rotated between the first orientation and the second orientation to cause the first and second tubes to approximate each other along the axis defined by the first and second tubes.

54. The system of claim 52 wherein the suture shuttle is mounted to the tool with the first opening mounted over the first tube, the second opening mounted over the second tube, and a longitudinal segment of the ribbon between the first and second openings extending down a first side of the tool, through the eyelet, and up a second slide of the tool.

55. The system of claim 54 wherein outer surfaces of the first and second tubes are of a size and shape larger than the openings in the suture shuttle when the openings are in an unstressed condition and wherein each opening in the suture shuttle is mounted over the outer surface of a respective one of the first and second tubes by resilient deformation of the opening into a stressed condition.

56. The system of claim 55 further comprising:
a cap adapted to be removably mounted on the handle, wherein the wire loop is attached to the cap.

57. The system of claim 51 wherein:
the suture shuttle is mounted to the tool with the first opening mounted over the first tube, the second opening mounted over the second tube, and a longitudinal segment of the ribbon between the first and second openings extending down a first side of the tool, through the eyelet, and up a second slide of the tool:
the bone anchor comprises:
a main anchor body defining a longitudinal axis, a receiving formation, and a formation for accepting a driving tool for driving the anchor main body into a bone;
an eyelet pin defining a longitudinal axis and bearing the eyelet, wherein the eyelet pin is longitudinally insertable in the receiving formation from an open position in which a suture may pass freely through the eyelet to a closed position in which a suture passing through the passage in the eyelet pin would be securely held in the eyelet;
the driving tool shaft has bore therein extending from an opening in the proximal end of the tool to an opening in the distal end of the tool, the distal end of the shaft frangibly attached to the main anchor body;
a rod disposed within the bore of the shaft having a distal end and a proximal end, the distal end of the rod abutting the eyelet pin of the bone anchor; and
a nut disposed between the shaft and the rod, the nut having a proximal end and a distal end and further bearing screw threads and being threadedly engaged to the shaft such that rotation of the nut relative to the shaft will cause the nut to move longitudinally relative to the shaft, and the nut being movable into a longitudinal position with the distal end of the nut abutting the proximal end of the rod such that longitudinal distal movement of the nut relative to the shaft results in longitudinal distal movement of the rod relative to the shaft;
wherein the first and second openings in the handle comprise longitudinally oriented slots within which the first and second tubes can slide in the longitudinal direction of the tool and wherein the nut is movable into a longitudinal position wherein the proximal end of the nut abuts the first and second tubes and can force the first and second tubes proximally within the slots to tension the ribbon.

58. In a system for loading a suture through an eyelet in a suture anchor, the system comprising a suture anchor including an eyelet for accepting sutures therethrough; a tool for implanting the suture anchor, the tool having a distal end and a proximal end, the suture anchor attached to the distal end of the tool, a handle at the proximal end of the tool and a shaft extending longitudinally between the proximal end and the distal end of the tool, at least one aperture disposed on the handle, the at least one aperture comprising an outer surface and an inner opening, and a suture shuttle comprising a ribbon of resilient material having a first longitudinal and a second longitudinal end and at least one opening in the ribbon for accepting a suture therethrough, wherein the resilient material of the ribbon biases the at least one opening into a first condition in which a clearance defined by the at least one opening is a first, smaller size and wherein the resilient material of the ribbon may be flexed by application of a force so as to place the at least one opening in a second condition in which the clearance defined by the at least one opening is a second, larger size, where, upon removal of the force, the at least one opening returns to the first condition, a method of mounting the suture shuttle to the tool, the method comprising the steps of:
passing the ribbon through the eyelet;
placing the at least one opening over the outer surface of the at least one aperture on the handle by forcing the first at least one opening into the second condition wherein the bias of the at least one opening causes the at least one opening to engage the outer surface of the at least one aperture in an interference fit.

59. The method of claim 58 wherein the at least one aperture comprises first and second apertures and the at least one opening comprises first and second openings spaced longitudinally from each other along a length of the ribbon and wherein the placing comprises placing the first opening over the first aperture and placing the second opening over the second aperture.

60. The method of claim 58 wherein the tool further comprises a nut threadedly engaged to the shaft of the tool whereby the nut may be moved longitudinally relative to the at least one aperture in the handle and wherein the at least one aperture is slidably mounted within at least one longitudinally oriented slot on the handle, the at least one aperture being positioned proximally of the nut such that the nut may contact the at least one aperture to force the at least one aperture proximally to the handle, the method further comprising:
prior to placing the at least one opening over the outer surface of the at least one aperture, positioning the nut in a first position relative to the handle, in which position the at least one aperture may be slid distally within the at least one slot in the handle to a first position in which the at least one opening may be mounted on the outer surface of the at least one aperture with the suture shuttle in a tension free condition;
prior to placing the at least one opening slit over the outer surface of the at least one aperture, positioning the first and second at least one aperture in the first position; and
after placing the at least one opening over the outer surface of the at least one aperture, moving the nut proximally relative to the handle to push the at least one aperture proximally on the handle to tension the suture shuttle.

61. The method of claim 59 wherein the tool further comprises a nut threadedly engaged to the shaft of the tool whereby the nut may be removed longitudinally relative to the first and second apertures in the handle and wherein the first and second apertures are slidably mounted within at least one longitudinally oriented slot on the handle, the first and second apertures being positioned proximally of the nut such that the nut may contact the first and second apertures to force then proximally relative to the handle, the method further comprising:
prior to placing the second opening over the outer surface of the second aperture, positioning the nut in a first position relative to the handle, in which position the first and second apertures may be positioned distally within the slots in the handle to a first position in which the suture shuttle may be mounted into the outer surfaces of the first and second apertures while in a tension free condition;
prior to placing the second opening over the outer surface of the second aperture, positioning the first and second apertures in the first position; and
after placing the first opening over the outer surface of the first aperture and placing the second opening over the outer surface of the second aperture, moving the nut proximally relative to the handle to force the apertures proximally to tension the suture shuttle.

62. The method of claim 61 wherein the first and second apertures are formed of a tube extending between and through the first and second slots in the handle, the tube having first and second longitudinal ends, the first and second longitudinal ends comprising the outer surfaces of the first and second apertures, respectively, the tube defining an open passageway therein extending between a first opening in the first longitudinal end of the tube and a second opening in the second longitudinal end of the tube, the first and second openings in the tube comprising the inner openings of the first and second apertures, respectively, and wherein the nut pushes the tube proximally relative to the handle by contacting the tube.

63. The method of claim 59 wherein the first and second apertures comprise first and second coaxial tubes formed in a clip, each of the first and second coaxial tubes defining an outer surface and an inner passage, the first and second substantially coaxial tubes disposed through the first and second slots in the handle, respectively, and the clip further comprising a resilient lever segment extending from and interconnecting the first and second tubes, wherein the nut pushes the first and second tubes proximally relative to the handle.

64. In a system for implanting a suture anchor in an anatomical feature, and loading a suture through an eyelet in the suture anchor, the system comprising a suture anchor including an eyelet, a tool for implanting the suture anchor, the suture anchor removably attached to a distal end of the tool, at least one aperture disposed in a proximal end of the tool, the at least one aperture comprising an outer surface and an inner opening, and a suture shuttle comprising a ribbon of resilient material having at least one opening for accepting a suture therethrough, wherein the resilient material of the ribbon biases the at least one opening into a first condition in which a clearance defined by the at least one opening is a first, smaller size and wherein the resilient material of the ribbon may be flexed by application of a force so as to place the at least one opening in a second condition in which the at least one opening defined by the slit is a second, larger size, where, upon the removal of the force, the at least one opening returns to the first condition, wherein the suture shuttle is mounted to the tool with the at least one opening mounted over the outer surface of the at least one aperture, and the ribbon extending through the eyelet, a method of loading a suture through the eyelet of the suture anchor, the method comprising:
inserting a suture through the inner opening of the at least one aperture;
removing the at least one opening from over the at least one aperture, whereby the at least one opening returns to the first condition, thereby locking the suture in the at least one opening; and
pulling on the suture shuttle until the at least one opening passes through the eyelet.

65. The method of claim 64 wherein the removing comprises drawing the ribbon away from the at least one aperture in a direction radially outwardly from the tool.

66. The method of claim 64 wherein the at least one aperture comprises first and second apertures and the at least one opening comprises first and second openings spaced longitudinally from each other along a length of the ribbon and wherein the first and second apertures are formed in a tube extending between and through first and second windows in the tool, the tube having an outer surface and defining an open passageway therein extending between a first opening in a first longitudinal end of the tube and a second opening in a second longitudinal end of the tube, the outer surface of the tube comprising the outer surfaces of the first and second apertures, the open passageway in the tube comprising the inner openings of the first and second apertures, wherein the removing comprises folding the tube so as to draw the first and second apertures radially inward through the windows in the tool until the tube is free of the windows.

67. The method of claim 66 wherein the tube further comprises a third opening intermediate the first and second ends of the tube and a weakened segment about which the tube will fold when subjected to a transverse force, wherein the removing comprises folding the tube about the weakened segment.

68. The method of claim 67 further comprising a wire having a first end attached to the tube near the weakened segment and a second, free end and wherein the removing comprises pulling on the free end of the wire to apply a transverse force to the tube to cause it to bend.

69. The method of claim 64 wherein the tool further comprises at least one opening and the at least one aperture comprises at least one tube formed in a clip, the at least one tube disposed through at least one window in the handle, and the clip further comprising a lever connected to the at least one tube, wherein the tool further comprises at least one cam surface positioned and shaped to act upon the clip to resiliently deform the clip as it is rotated between a first orientation and a second orientation to cause the at least one tube to translate along a longitudinal axis of the at least one tube out of the at least one window in the tool, wherein the removing comprises rotating the clip from the first orientation to the second orientation.

70. The system of claim 69 wherein the clip is resilient and is attached to the handle such that the at least one tube is resiliently biased to maintain the at least one tube in a position in which the at least one tube is disposed through the at least one window in the tool and wherein the clip may be manipulated to overcome the resilient bias to slide the at least one tube out of the at least one window in the tool.

71. The system of claim 70 wherein the clip further comprises a lever operatively connected to the at least one tube and the proximal end of the tool further comprises at least one cam surface and wherein the clip can be manipulated to cause the lever to ride on the cam surface to cause the at least one tube to slide out of the at least one window in the tool.

72. The system of claim 71 wherein the clip can be manipulated to cause the lever to ride on the cam surface to cause the at least one tube to translate substantially along the longitudinal axis of the at least one tube out of the at least one window in the tool.

73. The system of claim 72 wherein the manipulation of the clip that causes the lever to ride on the cam surface comprises rotation about a longitudinal axis of the at least one tube.

74. The system of claim 72 wherein the at least one tube comprises first and second coaxial tubes and the at least one passage in the tool comprises first and second aligned windows.

75. The method of claim 74 wherein the translating comprises the first and second coaxial tubes approximating each other along the axis defined by the first and second tubes.

76. The method of claim 64 wherein the at least one aperture comprises first and second apertures and the at least one opening comprises first and second openings spaced longitudinally from each other along a length of the ribbon and wherein the first and second apertures are formed in an insert extending between and through first and second slots in the tool, the slots extending and open to a proximal end of the tool, the apertures on the insert each comprising a channel structure having an outer surface with an opening therein defining an open channel, the outer surfaces of the channel structure comprising the outer surfaces of the first and second apertures and the open channels in the partial tubes comprising the inner opening of the first and second apertures, wherein:
the inserting a suture comprises inserting the suture through the open channel in a direction radially inward of the tool; and
the removing comprises pulling on a portion of the suture that passes through the open channel so as to cause the suture to pass out of open channel through the opening in the channel structure and bear on the opening of the suture shuttle so as to urge the suture shuttle free of the channel structure with the suture still in the opening in the suture shuttle.

77. The method of claim 76 wherein the insert is slidably mounted within a longitudinally oriented slot on the handle and open to a proximal end of the handle and wherein the method further comprises:
withdrawing the insert from the tool through the longitudinally oriented slot after the removing.

78. In a system for implanting a suture anchor in an anatomical feature, and loading a suture through an eyelet in the suture anchor, the system comprising a suture anchor including an eyelet, a tool for implanting the suture anchor, the suture anchor removably attached to a distal end of the tool, at least one aperture disposed in a proximal end of the tool, the at least one aperture comprising an outer surface and an inner opening, and a suture shuttle comprising a ribbon of resilient material at least one opening for accepting a suture therethrough, wherein the resilient material of the ribbon biases the at least one opening into a first condition in which a clearance defined by the at least one opening is a first, smaller size and wherein the resilient material of the ribbon may be flexed by application of a force so as to place the at least one opening in a second condition in which the clearance of the at least one opening is a second, larger size, where, upon removal of the force, the at least one opening returns to the first condition, wherein the suture shuttle is mounted to the tool with the at least one opening mounted over the at least one aperture, and the ribbon extending through the eyelet, and a suture loading mechanism comprising a wire loop disposed through the inner opening of the at least one aperture with a first portion of the wire loop extending from a first end of the inner opening and a second portion of the wire loop extending from a second end of the inner opening, a method of loading a suture through the eyelet of the suture anchor, the method comprising:
inserting a suture through the first portion of the wire loop;
pulling on the second portion of the wire loop to pull the wire loop completely through and out of the second end of the inner opening of the aperture carrying the suture with it;
removing the at least one opening of the suture shuttle from the outer surface of the at least one aperture, whereby the at least one opening returns to the first condition thereby locking the suture in the at least one opening; and
pulling on the suture shuttle to cause the suture to pass through the eyelet.

79. The method of claim 78 wherein the suture comprises a free end, the method further comprising:
holding the free end of the suture from the first end of the inner opening of the at least one aperture during the pulling to assure that it does not slide through the wire loop during the pulling; and
after the wire loop has been pulled through the at least one aperture, pulling the free end of the suture through the inner opening of the aperture from the second end of the aperture.

80. The method of claim 79 further comprising:
after the free end of the suture has been pulled through the at least one aperture from the second end, freeing the suture from the wire loop; and
after removing, pulling the suture back through the at least one aperture from the first end of the at least one aperture.

81. The method of claim 78 wherein the system further comprises a cap adapted to be removably mounted on the proximal end of the tool, wherein the wire loop is attached to the cap and wherein the pulling comprises removing the cap from the handle.

82. The method of claim 78 wherein the at least one aperture comprises first and second apertures and the first and second apertures are formed in a tube extending between and through first and second windows in the handle, the tube having an outer surface and defining an open passageway therein extending between a first opening in a first longitudinal end of the tube and a second opening in a second longitudinal end of the tube, the outer surface of the tube comprising the outer surfaces of the first and second apertures, the open passageway in the tube comprising the inner openings of the first and second apertures, a third opening in the tube intermediate the first and second ends of the tube, wherein the wire loop extends through the tube between the third opening and the first opening, the first portion of the wire loop extending out of the first opening and the second portion of the wire loop extending out of the third opening, and wherein the pulling comprises pulling on the second portion of the wire loop from the third opening.

83. The method of claim 82 wherein the tube further comprises:
a weakened segment about which the tube will deform when subjected to a transverse force and wherein the wire loop is further attached to the tube through a wire segment in a manner such that the wire loop may be substantially removed from the tube through the third opening while still attached to the tube, and may be further pulled to apply a transverse force to the tube to cause it to bend about the weakened segment and be removed from the handle in a direction transverse to a longitudinal axis of the tube and wherein the removing comprises pulling on the suture loading mechanism with sufficient force to fold the tube about the weakened portion.

84. A bone anchor device comprising:
a main anchor body defining a longitudinal axis and having an external formation for engaging the main anchor body to a bone, a central bore and at least one protrusion extending into the central bore; and
a central pin disposed in the central bore and having a longitudinal axis parallel to the longitudinal axis of the main anchor body, the central pin having a proximal end and a distal end; and
an eyelet pin defining a longitudinal axis and having a proximal end and a distal end along the longitudinal axis, a passage substantially transverse to the longitudinal axis through which a length of suture can be threaded, and a distal longitudinal bore running between and open to each of the transverse passage and the distal end of the eyelet pin, wherein the distal longitudinal bore of the eyelet pin forms an interference fit with the central pin, the eyelet pin further comprising at least one protrusion extending outwardly from the eyelet pin and adapted to engage the protrusion of the main anchor body so as to prevent the eyelet pin from rotating about its longitudinal axis beyond a predetermined point in the central bore in a first direction and to permit the eyelet pin to rotate in a second direction about its longitudinal axis;
wherein the eyelet pin is longitudinally insertable in the receiving formation into a closed position in which a suture passing through the passage in the eyelet pin would be securely held in the passage.

85. The bone anchor device of claim 84 wherein one of the protrusion on the eyelet pin and the protrusion on the anchor main body comprises a series of ratchets and the other of the protrusion on the eyelet pin and the protrusion on the anchor main body comprises a pawl.

86. The bone anchor device of claim 85 wherein at least one of the ratchet and pawl are formed of a material with resilience that permits the ratchet and pawl to rotate past each other in the second direction.

87. The bone anchor device of claim 85 wherein the bone anchor device has an open position in which a suture would be freely slidable through the passage and wherein, in the open position, the central pin does not intrude into the transverse passage of the eyelet pin sufficiently to trap sutures in the transverse passage and wherein the eyelet pin is translatable relative to the central pin along their respective longitudinal axes from the open position to the closed position, in which closed position the proximal end of the central pin extends into the transverse passage sufficiently so that a suture in the transverse passage would be trapped between the central pin and the eyelet pin.

88. The bone anchor device of claim 87 further comprising:
a resilient locking ring captured in the central bore and having an inner diameter and an outer diameter;
wherein the eyelet pin further comprises at least a first ramp formation on an outer surface thereof defining a diameter greater than the inner diameter of the locking ring when in an unbiased state, the first ramp formation adapted to cooperate with the locking ring to capture the eyelet pin in the main anchor body.

89. The bone anchor device of claim 88 wherein the first ramp formation is adapted to impart a spreading force on the locking ring when the eyelet pin is driven distally into the receiving formation whereby, upon application of sufficient force for the first ramp formation to bias the locking ring to a diameter greater than the diameter defined by the first ramp formation, the first ramp formation can traverse the locking ring to a position distal of the locking ring, whereupon the locking ring will return to its unbiased state, thereby locking the eyelet pin in the main anchor body.

90. The bone anchor device of claim 89 wherein, in the open position, the first ramp formation is positioned distal of the locking ring.

91. The bone anchor device of claim 90 wherein the eyelet pin further comprises a second ramp formation on the outer surface thereof proximal of the first ramp formation and defining a diameter greater than the inner diameter of the locking ring when in an unbiased state, the second ramp formation positioned such that, when the device is in the open state, the second ramp formation is proximal of the locking ring, and, when the device is in the closed state, the second ramp formation is distal of the locking ring.

92. A bone anchor device comprising:
a main anchor body defining a longitudinal axis and having an external formation for engaging the main anchor body to a bone and a central bore and at least one protrusion extending into the central bore; and
a central pin disposed in the central bore and having a longitudinal axis parallel to the longitudinal axis of the main anchor body, the central pin having a proximal end and a distal end; and
an eyelet pin defining a longitudinal axis and having a proximal end and a distal end along the longitudinal axis, a passage substantially transverse to the longitudinal axis through which a length of suture can be threaded, and a distal longitudinal bore running between and open to each of the transverse passage and the distal end of the eyelet pin, wherein the distal longitudinal bore of the eyelet pin forms an interference fit with the central pin;
wherein the eyelet pin is longitudinally insertable in the central bore into a closed position in which a suture passing through the passage in the eyelet pin would be securely held in the passage, wherein there are a plurality of closed positions at different heights at which the eyelet pin may be captured in the central bore.

93. The bone anchor device of claim 92 wherein the bone anchor device has an open position in which a suture would be freely slidable through the passage and wherein, in the open position, the central pin does not intrude into the transverse passage of the eyelet pin sufficiently to trap a suture in the transverse passage and wherein the eyelet pin is translatable relative to the central pin along their respective longitudinal axes from the open position to the closed position, in which closed position the proximal end of the central pin extends into the transverse passage sufficiently so that a suture in the transverse passage would be trapped between the central pin and the eyelet pin.

94. The bone anchor device of claim 93 further comprising:
a resilient locking ring captured in the central bore and having an inner diameter and an outer diameter;
wherein the eyelet pin further comprises a plurality of ramp formations on an outer surface thereof, each defining a diameter greater than the inner diameter of the locking ring when in an unbiased state, and each ramp formation adapted to cooperate with the locking ring to capture the eyelet pin in the main anchor body at a one of the plurality of different heights.

95. The bone anchor device of claim 94 wherein the ramp formations are adapted to impart a spreading force on the locking ring when the eyelet pin is driven distally into the receiving formation whereby, upon application of sufficient force for the ramp formations to bias the locking ring to a diameter greater than the diameter defined by the first ramp formation, the ramp formations can traverse the locking ring to a position distal of the locking ring, whereupon the locking ring will return to its unbiased state, thereby locking the eyelet pin in the main anchor body.

96. The bone anchor device of claim 95 wherein at least a multiplicity of ramp formations are positioned such that the device is in the closed state when any of the multiplicity of ramp formations is distal of the locking ring.
